(19) United States
(12) Patent Application Publication
(10) Pub. No.: US 2021/0208162 A1
(43) Pub. Date: Jul. 8, 2021

(54) DOWN SYNDROME BIOMARKERS AND USES THEREOF

(71) Applicant: Global Down Syndrome Foundation, Denver, CO (US)

(72) Inventor: Thomas BLUMENTHAL, Boulder, CO (US)

(21) Appl. No.: 16/071,335

(22) PCT Filed: Jan. 19, 2017
(86) PCT No.: PCT/US17/14122

§ 371 Date: Jul. 19, 2018

Related U.S. Application Data
(60) Provisional application No. 62/280,509, filed on Jan. 19, 2016.

(51) Int. Cl. G01N 33/68

(52) U.S. Cl. CPC G01N 33/6893; G01N 2800/52; G01N 2800/387

(57) ABSTRACT

The present invention provides novel biomarkers associated with Down syndrome. In particular, biomarkers identified from a proteome analysis of blood samples collected from affected individuals are disclosed that can be used to evaluate the disease spectrum of each individual with Down syndrome. Methods of using such biomarkers to develop diagnostics and therapeutics for prognosis and treatment of the conditions and diseases accompanying Down syndrome, and to develop diagnostics and therapeutics for prognosis and treatment of conditions and diseases prevalent in typical individuals but rare in individuals with Down syndrome are also described.

### CROSS-REFERENCE TO RELATED APPLICATIONS

This application claims the benefit of priority to U.S. provisional application Ser. No. 62/280,509, filed Jan. 19, 2016, which is herein incorporated in its entirety.

### TECHNICAL FIELD

The present invention provides novel biomarkers associated with Down syndrome. In particular, biomarkers identified from a proteome analysis of blood samples collected from affected individuals are disclosed that are used to evaluate the disease spectrum of each individual with Down syndrome. Methods of using such biomarkers to develop diagnostics and therapeutics for prognosis and treatment of the conditions and diseases accompanying Down syndrome, and to develop diagnostics and therapeutics for prognosis and treatment of conditions and diseases prevalent in typical individuals but rare in individuals with Down syndrome are also described.

### BACKGROUND

Down syndrome occurs when an individual has a full or partial extra copy of chromosome 21. The extra genetic material affects development and causes the characteristics associated with Down syndrome. A few of the common physical traits of Down syndrome are low muscle tone, small stature, an upward slant to the eyes, and a single deep crease across the center of the palm. However, individuals with Down syndrome may possess these characteristics to different degrees or not have them at all.

One in every 691 babies in the United States is born with Down syndrome, making Down syndrome the most common genetic condition. Approximately 450,000 Americans have Down syndrome and about 6,000 babies with Down syndrome are born in the United States each year.

There are three types of Down syndrome: trisomy 21 (nondisjunction), translocation and mosaicism. Nondisjunction occurs when there is an error in cell division at the gamete stage. Prior to or at conception, a pair of chromosome 21 in either the sperm or the egg fails to separate. Upon fusion of egg and sperm, an embryo results with three copies of chromosome 21 instead of the usual two. As the embryo develops, the extra chromosome is replicated in every cell of the body. This type of Down syndrome, which accounts for 95% of cases, is called trisomy 21.

Translocation accounts for about 4% of cases of Down syndrome. In translocation, the total number of chromosomes in the cells remains 46; however, an additional full or partial copy of chromosome 21 attaches to another chromosome, usually chromosome 14. The presence of the extra full or partial chromosome 21 causes the characteristics of Down syndrome.

Mosaicism (or mosaic Down syndrome) is diagnosed when there is a mixture of two types of cells, some containing the usual 46 chromosomes and some containing 47. Those cells with 47 chromosomes contain an extra chromosome 21. Mosaicism is the least common form of Down syndrome and accounts for only about 1% of all cases of Down syndrome.

Certain conditions and/or diseases are more common among people with Down syndrome compared to unaffected individuals. For example, individuals with Down syndrome have higher incidence of Alzheimer's, cognitive impairment, epilepsy, leukemia, diabetes, autism, congenital heart defects, celiac disease, thyroid dysfunction, autoimmune disorders, vision problems, hearing problems, intestinal atresia and sleep apnea. Conversely, it has also been observed that individuals with Down syndrome can also have reduced incidence of other conditions and/or diseases that are more prevalent in typical individuals such as heart disease, cancer, stroke, coronary artery disease, atherosclerosis, hypertension and angiopathies (e.g. diabetic retinopathies). Trisomy 21 is required but not sufficient to cause the various conditions and/or diseases associated with Down syndrome. In addition, the set of conditions and/or diseases associated with a given individual with Down syndrome may be different from the set of conditions and/or diseases associated with another individual with Down syndrome. Thus understanding the causal factors of these conditions and/or diseases would inform development of diagnostics and therapeutics not only for individuals with Down syndrome but for the general population also.

Global analysis of genes, transcripts, proteins, metabolites, epigenetic alterations and microbiomes have allowed a large-scale look at a physiological system under specified conditions and over time. The result of this analysis can identify biomarkers associated with a particular condition or disease.

A biomarker is a characteristic that is objectively measured and evaluated as an indicator of normal biologic processes, pathogenic processes, or pharmacologic responses to a therapeutic intervention. The goal in biomarker identification is an empiric interim readout that is directly related to clinical endpoints, i.e., a surrogate endpoint for disease progression and/or treatment effect. There are several potential advantages of a biomarker of disease severity or a network of biomarkers including: (1) replacement of a distal endpoint with a proximal endpoint, potentially shortening the development time of new therapeutic modalities, (2) more frequent and facile measurement, (3) increased precision, (4) increased measured dynamic range of a disease process or treatment effect compared to clinical metrics, (5) reduction in sample size requirements for clinical studies, and (6) expedited decisions concerning the efficacy and validity of therapeutic interventions. Thus, the development of biomarkers will have important implications in therapeutics development timelines and more efficiently allocate patient resources into studies with the greatest probability of success.

There is a need to understand Down syndrome at a more granular level in order to predict or assess each affected individual's disease spectrum, to develop diagnostics and therapeutics for treatment of the conditions and diseases accompanying Down syndrome and to develop diagnostics and therapeutics for treatment of conditions and diseases prevalent in typicals but rare in individuals with Down syndrome. This disclosure provides biomarkers for Down syndrome and methods of using such biomarkers.

### SUMMARY OF THE INVENTION

The present invention is based, in part, on the discovery of a panel of biomarkers in the blood from a wide range of individuals with Down syndrome that differ between individuals with Down syndrome and typical individuals.

One aspect of the invention provides a method of evaluating a condition or disease prevalent in an individual with Down syndrome compared to a typical individual, comprising: (a) measuring the level of at least one biomarker and/or the ratio of at least two biomarkers listed in Table 1 or Table 2 in a biological sample obtained from the individual with Down syndrome; and (b) comparing the measured level or ratio to a reference value or range of reference values, wherein the reference is one or more typical individuals, and wherein a change in the level of the at least one biomarker or the ratio of at least two biomarkers is indicative of the condition or disease prevalent in the individual with Down syndrome.

In one embodiment, at least one biomarker is a protein, peptide, polypeptide, polynucleotide, transcript, small molecule or microbiome profile. In another embodiment, at least one biomarker is a surrogate biomarker. In another embodiment, the biological sample is selected from the group consisting of saliva, tears, buccal swabs, nasal epithelium, skin, plasma, urine, blood and stool. In a further embodiment, the biological sample is blood.

In one embodiment, at least one biomarker is selected from Table 1 or Table 2. In another embodiment, the at least one biomarker is fibroblast growth factor receptor, epidermal growth factor receptor, platelet-derived growth factor receptor, neuropilin, matrix metalloproteinase, B2-microglobulin, Cystatin C, Annexin II, Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin. In another embodiment, the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, 06, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE.

In exemplary embodiments, at least one biomarker functions in a pathway selected from the group consisting of acute phase response signaling, the complement pathway and the prothrombin pathway. In one embodiment, the at least one biomarker functions in the acute phase response signaling. In another embodiment, the at least one biomarker is TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9) or 4-1BB ligand (TNFSF9). In one embodiment, the at least one biomarker functions in the complement pathway. In another embodiment, the at least one biomarker is C1QBP, C1R, C1S, C3, C6, C7, CFH or CFP. In one embodiment, the at least one biomarker functions in the prothrombin pathway. In another embodiment, the at least one biomarker is SerpinC1 (antithrombin III), MBL2 or F10 (Factor Xa).

In another embodiment, the measured level of at least one biomarker is indicative of the type and/or the severity of the condition or disease in the individual with Down syndrome. In another embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

Another aspect of the invention provides a method of evaluating the therapeutic efficacy of a therapeutic intervention for treating a condition or disease in an individual with Down syndrome comprising: (a) obtaining at least one initial biological sample from the individual at an initial time point, wherein the initial time point is prior to the administration of the therapeutic intervention; (b) obtaining at least one subsequent biological sample from the individual at a subsequent time point, wherein the subsequent time point is after the administration of the therapeutic intervention; (c) measuring the level of at least one biomarker listed in Table 1 or Table 2 in the initial and subsequent biological samples; and (d) comparing the level of at least one biomarker in the at least one initial biological sample to the level of at least one biomarker in at least one subsequent biological sample, wherein a change in the level of at least one biomarker is indicative of the efficacy of the therapeutic intervention as a treatment for the condition or disease in the individual with Down syndrome.

In one embodiment of the method of evaluating the therapeutic efficacy of a therapeutic intervention for treating a condition or disease in an individual with Down syndrome, at least one biomarker is a peptide, polypeptide, protein, polynucleotide, transcript, small molecule or microbiome profile. In another embodiment, at least one biomarker is a surrogate biomarker. In another embodiment, the biological sample is selected from the group consisting of saliva, tears, buccal swabs, nasal epithelium, skin, plasma, urine, blood and stool. In a further embodiment, the biological sample is blood.

In one embodiment, the at least one biomarker is selected from Table 1 or Table 2. In another embodiment, the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE.

In other embodiments, the at least one biomarker functions in a pathway selected from the group consisting of acute phase response signaling, the complement pathway and the prothrombin pathway. In one embodiment, the at least one biomarker functions in the acute phase response signaling. In another embodiment, the at least one biomarker is TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9) or 4-1BB ligand (TNFSF9). In one embodiment, the at least one biomarker functions in the complement pathway. In another embodiment, the at least one biomarker is C1QBP, C1R, C1S, C3, C6, C7, CFH or CFP. In one embodiment, the at least one biomarker functions in the prothrombin pathway. In another embodiment, the at least one biomarker is SerpinC1 (antithrombin III), MBL2 or F10 (Factor Xa).

In another embodiment, the therapeutic intervention is a compound or biologic selected from a compound or biologic library. In another embodiment, the condition or disease comprises Alzheimer's, cognitive impairment, epilepsy, leukemia, diabetes, autism, congenital head defects, celiac disease, thyroid dysfunction, autoimmune disorders, vision problems, hearing problems, intestinal atresia and/or sleep apnea.

Another aspect of the invention provides a method of confirming or refuting a diagnosis of a condition or disease in an individual with Down syndrome comprising: (a) measuring the level of at least one biomarker and/or the ratio of at least two biomarkers listed in Table 1 or Table 2 in a biological sample obtained from said individual; and (b) comparing the measured level or ratio to a reference value or range of reference values, wherein the diagnosis of the condition or disease in said individual is confirmed or refuted based on a change in the level of the at least one biomarker or the ratio of at least two biomarkers. In one embodiment of the method of confirming or refuting a diagnosis of a condition or disease in an individual with Down syndrome, the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSFS), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE. In another embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea

Another aspect of the invention provides a method of monitoring treatment of a condition or disease in an individual with Down syndrome in need thereof comprising: (a) obtaining at least one initial biological sample from the individual at an initial time point, wherein the initial time point is prior to the start of a therapeutic intervention protocol for the condition or disease; (b) obtaining at least one subsequent biological sample from the individual at a subsequent time point, wherein the subsequent time point is after the start of the therapeutic intervention protocol; (c) measuring the level of at least one biomarker or panel of biomarkers listed in Table 1 or Table 2 in the initial and subsequent biological samples; and (d) comparing the level of the at least one biomarker or panel of biomarkers in the at least one initial biological sample to the level of the at least one biomarker or panel of biomarkers in the at least one subsequent biological sample, wherein a change in the level of the at least one biomarker or panel of biomarkers is indicative of the efficacy of the therapeutic intervention protocol. In one embodiment, the method of monitoring treatment of a condition or' disease in an individual with Down syndrome in need thereof further comprises modifying or changing the therapeutic intervention protocol based on the level of one or more biomarkers. In one embodiment in the method of monitoring treatment of a condition or disease in an individual with Down syndrome in need thereof, the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B), TAJ (TNFRSFI9), DR3 (TNFRSF25), 4-1BB (TNFRSF9), 4-1BB ligand (TNFSF9), IMPDH1, IMPDH2, CIQBP, C1R, C1S, C3, C6, C7, CFH, OFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE. In another embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

Another aspect of the invention provides a biomarker kit comprising reagents for measuring one or more biomarkers listed in Table 1 or Table 2. In one embodiment, the one or more biomarkers is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSFIA), TNF sR-2 (TNFRSFIB), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE. In one embodiment, the kit further comprises a set of reference values to which the levels of the one or more biomarkers can be compared. In another embodiment, the reagents are adapted for measuring biomarkers in a blood sample. In another embodiment, the kit further comprises instructions for measuring said one or more biomarkers for diagnosing, evaluating level of severity, or monitoring progression of a condition or disease in an individual with Down syndrome. In another embodiment, the kit further comprises instructions for measuring said one or more biomarkers for monitoring the efficacy of a therapeutic intervention in an individual with Down syndrome having a condition or disease. In another embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

Another aspect of the invention provides a method for evaluating a sample from an individual with Down syndrome for a condition or disease, comprising: preparing a biomarker profile from a biological sample obtained from the individual, and determining the presence or absence of a biomarker signature indicative of the condition or disease, the biomarker profile comprising the level, abundance, or concentration of at least two biomarkers listed in Table 1 or Table 2. In one embodiment, the at least two biomarkers are selected from the group consisting of FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSFIA), TNF sR-2 (TNFRSFIB), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Co118A1, TrkB (NTRK2), TrkC (NTRK3), IGFBP6 and IgE. In one embodiment, the biological sample is a blood sample. In one embodiment of a method for evaluating a sample from an individual with Down syndrome for a condition or disease, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

Another aspect of the invention provides a method of evaluating a condition or disease prevalent in a typical individual but rare in an individual with Down syndrome, comprising: (a) measuring the level of at least one biomarker and/or the ratio of at least two biomarkers in a biological sample obtained from the typical individual; and (b) comparing the measured level or ratio to a reference value or range of reference values, wherein the reference is one or more individuals with Down syndrome, and wherein a change in the level of the at least one biomarker or the ratio of at least two biomarkers is indicative of the condition or disease prevalent in the typical individual. In one embodiment, the at least one biomarker is FGFR1, EGFR, ERBB3, ERBB4, PDGFR, NRP1, MMPI, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSFS), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE. In another embodiment, the condition or disease comprises heart disease, cancer, stroke, coronary artery disease, atherosclerosis, hypertension and/or angiopathies.

Another aspect of the invention provides a method for treating a condition or disease prevalent in an individual with Down syndrome, the method comprising administering an effective amount of a pharmaceutical composition to the individual, wherein the pharmaceutical composition reduces the expression or activity of a protein in Table 1 associated with the condition or disease prevalent in the individual with Down syndrome. In one embodiment, the protein is fibroblast growth factor receptor, neuropilin, matrix metalloproteinase, B2-microglobulin, Cystatin C, Annexin II, Notch protein, tumor necrosis factor (TNF) receptor, inosine 5′ monophosphate dehydrogenase, an insulin-like growth factor binding protein, Trefoil factor or endostatin. In another embodiment, the protein is FGFR1, NRP1, MMPI , B2-microglobulin (B2M), Cystatin C (CST3), Annexin II (ANXA2), Notch3, TNF sR-1 (TNFRSF1A), TNF sR-2 (TNFRSF1B), TAJ (TNFRSF19), DR3 (TNFRSF25), IMPDH1, IMPDH2, TFF3, TFF1, Col18A1 or IGFBP6. In a further embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

Another aspect of the invention provides a method for treating a condition or disease prevalent in an individual with Down syndrome, the method comprising administering an effective amount of a pharmaceutical composition to the individual, wherein the pharmaceutical composition increases the expression or activity of a protein in Table 2 associated with the condition or disease prevalent in the individual with Down syndrome. In one embodiment, the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE. In another embodiment, the protein is EGFR, ERBB3, ERBB4, PDGFR, Notch1, CD30 (TNFRSF8), CD30 ligand (TNFSFS), OPG (TNFRSF11B), 4-1BB (TNFRSF9), 4-1BB ligand (TNFSF9), C1QBP, C1R, C1S, C3, C6, C7, CFH, CFP, SerpinC1 (antithrombin III), MBL2, F10 (Factor Xa), BMP7, NOG, DKK1, DKK4, SHH, TrkB (NTRK2), TrkC (NTRK3) or IgE. In a further embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

Another aspect of the invention provides a method for treating a condition or disease prevalent in a typical individual, the method comprising administering an effective amount of a pharmaceutical composition to the individual, wherein the pharmaceutical composition increases the expression or activity of a protein in Table 1 associated with the condition or disease prevalent in the typical individual. In one embodiment, the protein is fibroblast growth factor receptor, neuropilin, matrix metalloproteinase, B2-microglobulin, Cystatin C, Annexin II, Notch protein, tumor necrosis factor (TNF) receptor, inosine 5′ monophosphate dehydrogenase, an insulin-like growth factor binding protein, Trefoil factor or endostatin. In another embodiment, the protein is FGFR1, NRP1, MMPI , B2-microglobulin (B2M), Cystatin C (CST3), Annexin II (ANXA2), Notch3, TNF sR-1 (TNFRSF1A), TNF sR-2 (TNFRSF1B), TAJ (TNFRSF19), DR3 (TNFRSF25), IMPDH1, IMPDH2, TFF3, TFF1, Col18A1 or IGFBP6. In a further embodiment, the condition or disease comprises heart disease, cancer, stroke, coronary artery disease, atherosclerosis, hypertension and/or angiopathies.

Another aspect of the invention provides a method for treating a condition or disease prevalent in a typical individual, the method comprising administering an effective amount of a pharmaceutical composition to the individual, wherein the pharmaceutical composition decreases the expression or activity of a protein in Table 2 associated with the condition or disease prevalent in the typical individual, In one embodiment, the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE. In another embodiment, the protein is EGFR, ERBB3, ERBB4, PDGFR, Notch1, CD30 (TNFRSF8), CD30 ligand (TNFSF8), OPG (TNFRSF11B), 4-1BB (TNFRSF9), 4-1BB ligand (TNFSF9), C1QBP, C1 R, CIS, C3, C6, C7, CFH, CFP, SerpinC1 (antithrombin III), MBL2, F10 (Factor Xa), BMP7, NOG, DKK1, DKK4, SHH, TrkB (NTRK2), TrkC (NTRK3) or IgE. In a further embodiment, the condition or disease comprises heart disease, cancer, stroke, coronary artery disease, atherosclerosis, hypertension and/or angiopathies,

Another aspect of the invention provides a method for treating a condition or disease prevalent in an individual with Down syndrome comprising administering an effective amount of a pharmaceutical composition to the individual, wherein the expression or activity level of a protein in Table 1 is higher than a predetermined threshold level, and wherein the effective amount of the pharmaceutical composition reduces the expression or activity level of the protein in the individual, In one embodiment, the protein is fibroblast growth factor receptor, neuropilin, matrix metalloproteinase, B2-microglobulin, Cystatin C, Annexin II, Notch protein, tumor necrosis factor (TNF) receptor, inosine 5′ monophosphate dehydrogenase, an insulin-like growth factor binding protein, Trefoil factor or endostatin. In another embodiment, the protein is FGFR1, NRP1, MMP1, B2-microglobulin (B2M), Cystatin C (CST3), Annexin II (ANXA2), Notch3, TNF sR-1 (TNFRSF1A), TNF sR-2 (TNFRSF1B), TAJ (TNFRSF19), DR3 (TNFRSF25), IMPDH1, IMPDH2, TFF3, TFF1, Coll 8A1 or IGFBP6. In a further embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

Another aspect of the invention provides a method for' treating a condition or disease prevalent in an individual with Down syndrome comprising administering an effective amount of a pharmaceutical composition to the individual, wherein the expression or activity level of a protein in Table 2 is lower than a predetermined threshold level, and wherein the effective amount of the pharmaceutical composition increases the expression or activity level of the protein in the individual, In one embodiment, the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE. In another embodiment, the protein is EGFR, ERBB3, ERBB4, PDGFR, Notch1, CD30 (TNFRSF8), CD30 ligand (TNFSF8), OPG (TNFRSF11B), 4-1BB (TNFRSF9), 4-1BB ligand (TNFSF9), C1QBP, C1 R, C1S, C3, C6, C7, CFH, CFP, SerpinC1 (antithrombin III), MBL2, F10 (Factor Xa), BMP7, NOG, DKKI, DKK4, SHH, TrkB (NTRK2), TrkC (NTRK3) or IgE. In a further embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

Another aspect of the invention provides a method for treating a condition or disease prevalent in a typical individual comprising administering an effective amount of a pharmaceutical composition to the individual, wherein the expression or activity level of a protein in Table 1 is lower than a predetermined threshold level, and wherein the effective amount of the pharmaceutical composition increases the expression or activity level of the protein in the individual. In one embodiment, the protein is fibroblast growth factor receptor, neuropilin, matrix metalloproteinase, B2-microglobulin, Cystatin C, Annexin II, Notch protein, tumor necrosis factor (TNF) receptor, inosine 5′ monophosphate dehydrogenase, an insulin-like growth factor binding protein, Trefoil factor or endostatin. In another embodiment, the protein is FGFR1, NRP1, MMPI, B2-microglobulin (B2M), Cystatin C (CST3), Annexin II (ANXA2), Notch3, TNF sR-1 (TNFRSF1A), TNF sR-2 (TNFRSF1B), TAJ (TNFRSF19), DR3 (TNFRSF25), IMPDH1, IMPDH2, TFF3, TFF1, Col18A1 or IGFBP6. In a further embodiment, the condition or disease comprises heart disease, cancer, stroke, coronary artery disease, atherosclerosis, hypertension and/or angiopathies.

Another aspect of the invention provides a method for treating a condition or disease prevalent in a typical individual comprising administering an effective amount of a pharmaceutical composition to the individual, wherein the expression or activity level of a protein in Table 2 is higher than a predetermined threshold level, and wherein the effective amount of the pharmaceutical composition decreases the expression or activity level of the protein in the individual. In one embodiment, the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE. In another embodiment, the protein is EGFR, ERBB3, ERBB4, PDGFR, Notch1, CD30 (TNFRSF8), CD30 ligand (TNFSF8), OPG (TNFRSF11B), 4-1BB (TNFRSF9), 4-1BB ligand (TNFSF9), C1 QBP, C1R, C1S, C3, C6, C7, CFH, CFP, SerpinC1 (antithrombin III), MBL2, F10 (Factor Xa), BMP7, NOG, DKK1, DKK4, SHH, TrkB (NTRK2), TrkC (NTRK3) or IgE. In a further embodiment, the condition or disease comprises heart disease, cancer, stroke, coronary artery disease, atherosclerosis, hypertension and/or angiopathies.

Another aspect of the invention provides a method for treating a condition or disease prevalent in an individual with Down syndrome, the method comprising (a) measuring the expression or activity level of a protein in Table 1 in a sample obtained from the individual; (b) comparing the expression or activity level of the protein to a reference level in a normal control; and (c) treating the individual with an effective amount of a pharmaceutical composition if the expression or activity level of the protein is increased relative to the reference level, wherein the pharmaceutical composition reduces the expression or activity of the protein. In one embodiment, the method further comprises step (d) of measuring the expression or activity of the protein in the individual alter step (c) of treating the individual, wherein a decrease in the expression or activity of the protein is indicative that the patient is responsive to the treatment, In one embodiment, the protein is fibroblast growth factor receptor, neuropilin, matrix metalloproteinase, B2-microglobulin, Cystatin C, Annexin II, Notch protein, tumor necrosis factor (TNF) receptor, inosine 5′ monophosphate dehydrogenase, an insulin-like growth factor binding protein, Trefoil factor or endostatin. In another embodiment, the protein is FGFR1, NRP1, MMPI , B2-microglobulin (B2M), Cystatin C (CST3), Annexin II (ANXA2), Notch3, TNF sR-1 (TNFRSF1A), TNF sR-2 (TNFRSF1B), TAJ (TNFRSF19), DR3 (TNFRSF25), IMPDH1, IMPDH2, TFF3, TFF1, Col18A1 or IGFBP6. In a further embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

Another aspect of the invention provides a method for treating a condition or disease prevalent in an individual with Down syndrome, the method comprising (a) measuring the expression or activity level of a protein in Table 2 in a sample obtained from the individual; (b) comparing the expression or activity level of the protein to a reference level in a normal control; and (c) treating the individual with an effective amount of a pharmaceutical composition if the expression or activity level of the protein is decreased relative to the reference level, wherein the pharmaceutical composition increases the expression or activity of the protein. In one embodiment, the method further comprises step (d) of measuring the expression or activity of the protein in the individual after step (c) of treating the individual, wherein an increase in the expression or activity of the protein is indicative that the patient is responsive to the treatment, In one embodiment, the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE. In another embodiment, the protein is EGFR, ERBB3, ERBB4, PDGFR, Notch1, CD30 (TNFRSF8), CD30 ligand (TNFSF8), OPG (TNFRSF11B), 4-1BB (TNFRSF9), 4-1BB ligand (TNFSF9), C1QBP, C1R, C1S, C3, C6, C7, CFH, CFP, SerpinC1 (antithrombin III), MBL2, F10 (Factor Xa), BMP7, NOG, DKK1, DKK4, SHH, TrkB (NTRK2), TrkC (NTRK3) or IgE. In a further embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

Another aspect of the invention provides a method for treating a condition or disease prevalent in a typical individual, the method comprising (a) measuring the expression or activity level of a protein in Table 1 in a sample obtained from the individual; (b) comparing the expression or activity level of the protein to a reference level in a normal control; and (c) treating the individual with an effective amount of a pharmaceutical composition if the expression or activity level of the protein is decreased relative to the reference level, wherein the pharmaceutical composition increases the expression or activity of the protein. In one embodiment, the method further comprises step (d) of measuring the expression or activity of the protein in the individual after step (c) of treating the individual, wherein an increase in the expression or activity of the protein is indicative that the patient is responsive to the treatment. In one embodiment, the protein is fibroblast growth factor receptor, neuropilin, matrix metalloproteinase, B2-microglobulin, Cystatin C, Annexin II, Notch protein, tumor necrosis factor (TNF) receptor, inosine 5′ monophosphate dehydrogenase, an insulin-like growth factor binding protein, Trefoil factor or endostatin. In another embodiment, the protein is FGFR1, NRP1, MMPI , B2-microglobulin (B2M), Cystatin C (CST3), Annexin 11 (ANXA2), Notch3, TNF sR-1 (TNFRSF1A), TNF sR-2 (TNFRSF1B), TAJ (TNFRSF19), DR3 (TNFRSF25), IMPDH1, IMPDH2, TFF3, TFF1, Col18A1 or IGFBP6. In a further embodiment, the condition or disease comprises heart disease, cancer, stroke, coronary artery disease, atherosclerosis, hypertension and/or angiopathies.

Another aspect of the invention provides a method for treating a condition or disease prevalent in a typical individual, the method comprising (a) measuring the expression or activity level of a protein in Table 2 in a sample obtained from the individual; (b) comparing the expression or activity level of the protein to a reference level in a normal control; and (c) treating the individual with an effective amount of a pharmaceutical composition if the expression or activity level of the protein is increased relative to the reference level, wherein the pharmaceutical composition decreases the expression or activity of the protein. In one embodiment, the method further comprises step (d) of measuring the expression or activity of the protein in the individual after step (c) of treating the individual, wherein a decrease in the expression or activity of the protein is indicative that the patient is responsive to the treatment. In one embodiment, the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE. In another embodiment, the protein is EGFR, ERBB3, ERBB4, PDGFR, Notch1, CD30 (TNFRSF8), CD30 ligand (TNFSF8), OPG (TNFRSF11B), 4-1BB (TNFRSF9), 4-1BB ligand (TNFSF9), C1QBP, C1R, C1S, C3, C6, C7, CFH, CFP, SerpinC1 (antithrombin III), MBL2, F10 (Factor Xa), BMP7, NOG, DKK1, DKK4, SHH, TrkB (NTRK2), TrkC (NTRK3) or IgE. In a further embodiment, the condition or disease comprises heart disease, cancer, stroke, coronary artery disease, atherosclerosis, hypertension and/or angiopathies.

Another aspect of the invention provides a method for treating a condition or disease prevalent in an individual with Down syndrome, the method comprising (a) determining the expression or activity level of a protein in Table 1 in a first sample obtained from the individual; (b) determining the expression or activity level of the protein in a second sample from the individual, wherein the second sample is obtained after administration to the individual of an initial dosage of a pharmaceutical composition; and (c) administering an adjusted dosage of the pharmaceutical composition, wherein the adjusted dosage is greater than the initial dosage if the expression or activity level of the protein in the second sample is greater than a predetermined threshold level. In one embodiment, the protein is fibroblast growth factor receptor, neuropilin, matrix metalloproteinase, B2-microglobulin, Cystatin C, Annexin II, Notch protein, tumor necrosis factor (TNF) receptor, inosine 5′ monophosphate dehydrogenase, an insulin-like growth factor binding protein, Trefoil factor or endostatin, In another embodiment, the protein is FGFR1, NRP1, MMPI , B2-microglobulin (B2M), Cystatin C (CST3), Annexin II (ANXA2), Notch3, TNF sR-1 (TNFRSF1A), TNF sR-2 (TNFRSF1B), TAJ (TNFRSF19), DR3 (TNFRSF25), IMPDH1, IMPDH2, TFF3, TFF1, Col18A1 or IGFBP6. In a further embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

Another aspect of the invention provides a method for treating a condition or disease prevalent in an individual with Down syndrome, the method comprising (a) determining the expression or activity level of a protein in Table 2 in a first sample obtained from the individual; (b) determining the expression or activity level of the protein in a second sample from the individual, wherein the second sample is obtained after administration to the individual of an initial dosage of a pharmaceutical composition; and (c) administering an adjusted dosage of the pharmaceutical composition, wherein the adjusted dosage is greater than the initial dosage if the expression or activity level of the protein in the second sample is reduced relative to a predetermined threshold level. In one embodiment, the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE. In another embodiment, the protein is EGFR, ERBB3, ERBB4, PDGFR, Notch1, CD30 (TNFRSF8), CD30 ligand (TNFSF8), OPG (TNFRSF11B), 4-1BB (TNFRSF9), 4-1BB ligand (TNFSF9), C1QBP, C1R, C1S, C3, C6, C7, CFH, CFP, SerpinC1 (antithrombin III), MBL2, F10 (Factor Xa), BMP7, NOG, DKK1, DKK4, SHH, TrkB (NTRK2), TrkC (NTRK3) or IgE. In a further embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

Another aspect of the invention provides a method for treating a condition or disease prevalent in an individual with Down syndrome, the method comprising (a) administering a first dosage of a pharmaceutical composition; (b) determining the expression or activity level of a protein in Table 1 in a sample obtained from the individual; (c) determining whether the dosage of the pharmaceutical composition needs to be adjusted based on whether the expression or activity level of the protein falls within a target range, wherein an expression or activity level of the protein falling above the target range indicates that the dosage needs to be increased and an expression or activity level of the protein falling below a target range indicates that the dosage needs to be decreased, and (d) administering a second dosage of the pharmaceutical composition based on the determination in (c). In one embodiment, the protein is fibroblast growth factor receptor, neuropilin, matrix metalloproteinase, B2-microglobulin, Cystatin C, Annexin II, Notch protein, tumor necrosis factor (TNF) receptor, inosine 5′ monophosphate dehydrogenase, an insulin-like growth factor binding protein, Trefoil factor or endostatin. In another embodiment, the protein is FGFR1, NRP1, MMP1, B2-microglobulin (B2M), Cystatin C (CST3), Annexin II (ANXA2), Notch3, TNF sR-1 (TNFRSF1A), TNF sR-2 (TNFRSF1B), TAJ (TNFRSF19), DR3 (TNFRSF25), IMPDH1, IMPDH2, TFF3, TFF1, Col18A1 or IGFBP6. In a further embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea,

Another aspect of the invention provides a method for treating a condition or disease prevalent in an individual with Down syndrome, the method comprising (a) administering a first dosage of a pharmaceutical composition; (b) determining the expression or activity level of a protein in Table 2 in a sample obtained from the individual; (c) determining whether the dosage of the pharmaceutical composition needs to be adjusted based on whether the expression or activity level of the protein falls within a target range, wherein an expression or activity level of the protein falling below the target range indicates that the dosage needs to be increased and an expression or activity level of the protein falling above a target range indicates that the dosage needs to be decreased, and (d) administering a second dosage of the pharmaceutical composition based on the determination in (c), In one embodiment, the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE. In another embodiment, the protein is EGFR, ERBB3, ERBB4, PDGFR, Notch1, CD30 (TNFRSF8), CD30 ligand (TNFSF8), OPG (TNFRSF11B), 4-1BB (TNFRSF9), 4-1BB ligand (TNFSF9), C1CBP, C1R, C1S, C3, C6, C7, CFH, CFP, SerpinC1 (antithrombin III), MBL2, F10 (Factor Xa), BMP7, NOG, DKK1, DKK4, SHH, TrkB (NTRK2), TrkC (NTRK3) or IgE. In a further embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

Another aspect of the invention provides a method for treating a condition or disease prevalent in an individual with Down syndrome comprising measuring the expression or activity level of a protein in Table 1 or Table 2 in the individual upon administration a first amount of a pharmaceutical composition, administering a second amount of the pharmaceutical composition to the patient, wherein the second amount of the pharmaceutical composition is determined based on the expression or activity level of protein in Table 1 or Table 2. In one embodiment, the protein is fibroblast growth factor receptor, epidermal growth factor receptor, platelet-derived growth factor receptor, neuropilin, matrix metalloproteinase, B2-microglobulin, Cystatin C, Annexin II, Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin. In another embodiment, the protein is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE. In a further embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

Another aspect of the invention provides a method for treating a condition or disease prevalent in a typical individual comprising measuring the expression or activity level of a protein in Table 1 or Table 2 in the individual upon administration a first amount of a pharmaceutical composition, administering a second amount of the pharmaceutical composition to the patient, wherein the second amount of the pharmaceutical composition is determined based on the expression or activity level of protein in Table 1 or Table 2. In one embodiment, the protein is fibroblast growth factor receptor, epidermal growth factor receptor, platelet-derived growth factor receptor, neuropilin, matrix metalloproteinase, B2-microglobulin, Cystatin C, Annexin II, Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin. In another embodiment, the protein is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSFS), CD30 ligand (TNFSF8), OPG (TNFRSF11B), TAJ (TNFRSF19). DR3 (TNFRSF25), 4-1BB (TNFRSF9). 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG. DKK1, DKK4, SHH, TFF3, TFF1, Col18A1, TrkB (NTRK2), TrkC (NTRK3), IGFBP6 or IgE. In a further embodiment, the condition or disease comprises heart disease, cancer, stroke, coronary artery disease, atherosclerosis, hypertension and/or angiopathies.

### BRIEF DESCRIPTION OF DRAWINGS

Illustrative embodiments of the invention are illustrated in the drawings, in which:

FIG. 1 illustrates a scheme for sample collection and data analysis from 1000 individuals with Down syndrome and +500 typical controls. EMR=electronic medical records.

FIG. 2 illustrates a cross-sectional data collection.

FIG. 3 illustrates a longitudinal data collection.

FIG. 4A-4B illustrates the distribution of protein expression changes across the genome from a proteomics discovery study comparing individuals with Down syndrome and typical individuals. FIG. 4A shows the loge fold change in expression of 3624 proteins across the 22 autosomes and X and Y sex chromosomes. Gray and black dots represent non-significant protein expression changes; red dots represent significant protein expression changes at FDR<10%. FDR=false discovery rate. 97% of significant protein expression changes were on chromosomes other than chromosome 21, while 3% of significant protein expression changes were on chromosome 21. FIG. 4B is an enlargement of the distribution in FIG. 4A centered on chromosomes 19, 20, 21 and 22 to show that proteins on chromosome 21 tended to be upregulated, with some reaching statistical significance, but none were significantly downregulated.

FIG. 5A-5B illustrates that significant protein expression changes on chromosome 21 are all increases. FIG. 5A shows the loge fold change in expression of proteins on chromosome 20 compared to that on chromosome 21. Gray and black dots represent non-significant protein expression changes; red dots represent significant protein expression changes at FDR<10%. FDR=false discovery rate. FIG. 5B shows a distribution of log₂ fold change in expression of proteins on chromosomes other than chromosome 21 versus on chromosome 21 in a box plot diagram.

FIG. 6A-6C illustrates that trisomy 21 causes significant changes in the systemic proteome. FIG. 6A shows the p value (in −log₁₀) of 1129 proteins that were upregulated or downregulated in individuals with Down syndrome compared to typical individuals. The thresholds for p<0.05 and FDR<10% are indicated by the dashed lines. Each black dot represents an upregulated or downregulated protein, and epidermal growth factor receptor (EGFR) and fibroblast growth factor receptor 1 (FGFR1) proteins are circled. FDR=false discovery rate. FIG. 6B shows a distribution of EGFR protein expression as measured by relative fluorescence units (RFU) of aptamers (see Example 1) in typical individuals versus in individuals with Down syndrome in a box plot diagram. FIG. 6C shows a distribution of FGFR protein expression as measured by RFU of aptamers in typical individuals versus in individuals with Down syndrome in a box plot diagram.

FIG. 7 illustrates that trisomy 21 causes significant changes in the systemic proteome. Graphs show the p value (in −log₁₀) of proteins that were upregulated or downregulated in individuals with Down syndrome compared to typical individuals in the Discovery study, Validation study 1 and Validation study 2. The thresholds for p<0.05 and FDR<10% are indicated by the dashed lines. Each black dot represents an upregulated or downregulated protein. FGFR1, neuropilin (NRP1) and matrix metalloproteinase 1 (MMP1) are circled in each graph. FDR=false discovery rate.

FIG. 8A-8B illustrates that trisomy 21 causes reproducible changes in the systemic proteome. FIG. 8A shows the p value (in −log₁₀) of proteins that were upregulated or downregulated in individuals with Down syndrome compared to typical individuals in the Discovery study. Each dot represents an upregulated or downregulated protein. FIG. 8A, left panel, shows all proteins that were upregulated or downregulated in individuals with Down syndrome compared to typical individuals. FIG. 8A, middle panel, shows the proportion of these proteins (green dots) that were validated in both Validation study 1 and Validation study 2 with p<0.05. FIG. 8A, right panel, shows the proportion of these proteins (red dots) that were validated in either Validation study 1 or Validation study 2 with p<0.05. The thresholds for p<0.05 and FDR<10% are indicated by the dashed lines. FDR=false discovery rate, FIG. 8B shows a distribution of FGFR protein expression as measured by RFU in a box plot diagram for typical individuals versus for individuals with Down syndrome in the Discovery study, Validation study 1 and Validation study 2.

FIG. 9 illustrates that trisomy 21 causes massive changes in the systemic proteome. The left panel shows the p value (in −log₁₀) of proteins that were upregulated or downregulated in individuals with Down syndrome compared to typical individuals in the Discovery study. The expression of 782 proteins was significantly downregulated at a 5% FDR, while the expression of 561 proteins was significantly upregulated at a 5% FDR. Two proteins that are significantly upregulated, FGFR1 and MMP1, are circled. The right panel shows the p value (in −log₁₀) of proteins that were upregulated or downregulated in typical females compared to typical males in the Discovery study. The expression of three proteins was significantly downregulated at a 5% FDR and the expression of one protein was significantly upregulated at a 5% FDR, Circled are KLK3 (PSA), a protein that is significantly downregulated in females, and CGA (FSHa), a protein that is significantly upregulated in females,

FIG. 10 illustrates that most protein expression changes caused by trisomy 21 occur in the proteome associated with chromosomes other than chromosome 21. The left panel shows the p value (in —log₁₀) of proteins that were upregulated or downregulated in individuals with Down syndrome compared to typical individuals in the Discovery study. The expression of 782 proteins was significantly downregulated at a 5% FDR, while the expression of 561 proteins was significantly upregulated at a 5% FDR. Two proteins that are significantly upregulated, FGFR1 and MMP1, are circled. The right panel highlights the upregulated and downregulated proteins that are encoded by genes on chromosome 21 (red dots). None of the 50 proteins encoded by genes on chromosome 21 are downregulated, whereas 10 of the 50 are upregulated. Examples of significantly upregulated chromosome 21 proteins include Trefoil factor 3 (TFF3), Trefoil factor 1 (TFF1) and COL18A1 (endostatin) (FIG. 10, right panel, circled dots).

FIG. 11A-11B illustrates that protein levels can change with age in individuals with Down syndrome and in typical individuals. FIG. 11A shows the level of insulin-like growth factor binding protein 6 (IGBP6) as measured by RFU of aptamers as a function of age. FIG. 11B shows the level of ERBB3 binding protein as measured by RFU of aptamers as a function of age.

FIG. 12 illustrates the change in TFF3 protein expression as measured by RFU of aptamers as a function of age for typical individuals and individuals with Down syndrome (left panel) and distribution of TFF3 protein expression in a box plot diagram for typical individuals versus for individuals with Down syndrome (right panel).

FIG. 13 illustrates the distribution of FGFR1 (left panel) and platelet-derived growth factor receptor (PDGFR) (right panel) protein expression in box plot diagrams in individuals with Down syndrome and in typical individuals.

FIG. 14 depicts signaling pathways regulating pluripotency of stem cells.

FIG. 15 illustrates the distribution of BMP7 (left panel) and NOG (right panel) protein expression in box plot diagrams in individuals with Down syndrome and in typical individuals. BMP7 and NOG are signaling peptides involved in bone and limb development.

FIG. 16 illustrates the distribution of DKK1 (left panel) and DKK4 (right panel) Wnt inhibitor protein expression in box plot diagrams in individuals with Down syndrome and in typical individuals.

FIG. 17 illustrates the distribution of Sonic hedgehog (SHH) protein expression in box plot diagrams in individuals with Down syndrome and in typical individuals.

FIG. 18 illustrates the distribution of epidermal growth factor receptor (EFGR, ERBB3, ERBB4) protein expression in box plot diagrams in individuals with Down syndrome and in typical individuals.

FIG. 19 illustrates the distribution of TrkB and TrkC neurotrophin receptor protein expression in box plot diagrams in individuals with Down syndrome and in typical individuals.

FIG. 20 illustrates the distribution of expression for a number of proteins in the complement cascade in box plot diagrams for individuals with Down syndrome and for typical individuals.

FIG. 21 illustrates the distribution of expression for a number of proteins in the coagulation cascade in box plot diagrams for individuals with Down syndrome and for typical individuals.

FIG. 22 (left panel) illustrates the distribution of IgE protein expression in a box plot diagram in individuals with Down syndrome and in typical individuals, FIG. 22 (right panel) illustrates the percent of individuals who have Down syndrome (DS) or who are typical individuals as a function of IgE protein level (as measured by RFU of aptamers).

FIG. 23 illustrates the distribution of MMP1 protein expression in a box plot diagram in individuals with Down syndrome and in typical individuals.

FIG. 24 illustrates the distribution of expression for B2-microglobulin and Cystatin C, proteins associated with impaired kidney function, in box plot diagrams for individuals with Down syndrome and for typical individuals.

FIG. 25 illustrates the distribution of expression for Annexin II, an autocrine factor that heightens osteoclast formation and bone resorption, in box plot diagrams for individuals with Down syndrome and for typical individuals.

FIG. 26 illustrates the distribution of expression for inosine 5′ monophosphate dehydrogenases 1 and 2 (IMPDH1 and IMPDH2), which act in the rate limiting step for GTP synthesis, in box plot diagrams for individuals with Down syndrome and for typical individuals.

FIG. 27 illustrates the distribution of neuropilin (NRP1) protein expression in box plot diagrams for individuals with Down syndrome and for typical individuals. NRP1 is a receptor for vascular endothelial growth factor (VEGF) and semaphorin and has roles in axon guidance and angiogenesis.

FIG. 28 illustrates the distribution of Notch1 and Notch3 protein expression in box plot diagrams for individuals with Down syndrome and for typical individuals.

FIG. 29 illustrates the distribution of expression for a number of receptors and ligands associated with tumor necrosis factor,

FIG. 30 illustrates the acute response signaling pathway,

FIG. 31 illustrates the complement pathway,

FIG. 32 illustrates the intrinsic prothrombin activation pathway.

### DETAILED DESCRIPTION

Technology now allows non-assumptive data mining of the human nucleic acid (genome and transcriptome), polypeptide (proteome), small molecule (metabolome), epigenome and gut microflora (microbiome) repertoire. A functional consequence is that this technology can be used to generate a disease “signature” of biochemical markers which segregates with a condition and/or disease process or severity associated with Down syndrome. These biochemical changes are assumed to be specific to individuals with Down syndrome. This has important consequences in therapeutics discovery for Down syndrome. For instance, (1) it can lead to the generation of sensitive disease biomarkers that are functional surrogates for disease progression and/or therapeutic benefit, (2) it can lead to identification of previously unidentified targets or pathways critical in the manifestation of the disease, (3) it can support hypotheses of pathogenesis that confirm and/or extend our knowledge of the disease process, and (4) it can identify targets or pathways important for preventing or treating diseases in typical individuals.

A biomarker can be invaluable to make more efficient treatment trials at both early and late stages of clinical investigation. At present, it is possible to assess the success of an intervention only in cumbersome and expensive prolonged interventional studies. A biomarker has the potential to provide a “read-out” in early proof of concept studies, permitting the allocation of limited treatment trial resources to be focused only on the most promising compounds. In more advanced clinical testing, a biomarker can help expand the potential population of patients to include those too young or old, or too severely or mildly affected, to be eligible for clinical trials as they must presently be done using clinical outcomes. Moreover, biomarkers may lessen the need to design trials that include frequent burdensome travel to clinical centers. Finally, treatment-associated improvement with biomarkers can enhance the meaningfulness of other clinical outcomes. Thus, there is need in the art to identify biomarkers that can serve as intermediate read-outs of disease spectrum in individuals with Down syndrome.

The present invention is based, in part, on the identification of biomarkers in biological samples (e.g., blood and stool) from a broad range of individuals with Down syndrome that differ in individuals with Down syndrome compared to typical individuals. Accordingly, the present invention provides a method of evaluating a condition or disease prevalent in an individual with Down syndrome compared to a typical individual, comprising: (a) measuring the level of at least one biomarker and/or the ratio of at least two biomarkers listed in Table 1 or Table 2 in a biological sample obtained from the individual with Down syndrome; and (b) comparing the measured level or ratio to a reference value or range of reference values, wherein the reference is one or more typical individuals, and wherein a change in the level of the at least one biomarker or the ratio of at least two biomarkers is indicative of the condition or disease prevalent in the individual with Down syndrome.

In one embodiment, the at least one biomarker is a protein, peptide, polypeptide, polynucleotide, transcript, small molecule or microbiome profile. In another embodiment, the at least one biomarker is a surrogate biomarker. In another embodiment, the biological sample is selected from the group consisting of saliva, tears, buccal swabs, nasal epithelium, skin, plasma, urine, blood and stool. In a further embodiment, the biological sample is blood.

In one embodiment, the at least one biomarker selected from Table 1 or Table 2. In another embodiment, the at least one biomarker is fibroblast growth factor receptor, epidermal growth factor receptor, platelet-derived growth factor receptor, neuropilin, matrix metalloproteinase, B2-microglobulin, Cystatin C, Annexin II, Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin. In another embodiment, the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE.

In other embodiments, the at least one biomarker functions in a pathway selected from the group consisting of acute phase response signaling, the complement pathway and the prothrombin pathway. In one embodiment, the at least one biomarker functions in the acute phase response signaling. In another embodiment, the at least one biomarker is TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9) or 4-1BB ligand (TNFSF9). In one embodiment, the at least one biomarker functions in the complement pathway. In another embodiment, the at least one biomarker is C1QBP, C1R, C1S, C3, C6, C7, CFH or CFP. In one embodiment, the at least one biomarker functions in the prothrombin pathway. In another embodiment, the at least one biomarker is SerpinC1 (antithrombin III), MBL2 or F10 (Factor Xa).

In another embodiment, the measured level of the at least one biomarker is indicative of the type and/or the severity of the condition or disease in the individual with Down syndrome. In one embodiment, the condition or disease in the individual with Down syndrome comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea. Alternatively, the measured level of the at least one biomarker is indicative of the type of and/or the severity of and/or the propensity to be afflicted by the condition or disease in a typical individual. In one embodiment, the condition or disease in a typical individual comprises heart disease, cancer, stroke, coronary artery disease, atherosclerosis, hypertension and/or angiopathies.

As used herein, the singular forms “a,” “an” and “the” include plural references, unless the content clearly dictates otherwise, and are used interchangeably with “at least one” and “one or more.” Thus, reference to “a biomarker” includes mixtures of biomarkers, reference to “a molecule” includes mixtures of molecules, and the like.

As used herein, the term “about” represents an insignificant modification or variation of the numerical value such that the basic function of the item to which the numerical value relates is unchanged.

As used herein, the terms “altered”, “changed” or “significantly different” refer to a detectable change or difference from a reasonably comparable state, profile, measurement, or the like. One skilled in the art should be able to determine a reasonable measurable change. Such changes may be all or none. They may be incremental and need not be linear. They may be by orders of magnitude. A change may be an increase or decrease by 1%, 5%, 10%, 20%,30%, 40%, 50%, 60%, 70%, 80%, 90%, 95%, 99%, 100%, or more, or any value in between 0% and 100%. Alternatively the change may be 1-fold, 1.5-fold 2-fold, 3-fold, 4-fold, 5-fold or more, or any values in between 1-fold and five-fold. The change may be statistically significant with a p value of 0.1, 0.05, 0.001, or 0.0001. The change may be statistically significant with a q value of less than 0.05.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

As used herein, the terms “biological sample”, “sample”, and “test sample” are used interchangeably to refer to any material, biological fluid, tissue, or cell obtained or otherwise derived from an individual. This includes blood (including whole blood, leukocytes, peripheral blood mononuclear cells, buffy coat, plasma, and serum), sputum, tears, mucus, nasal washes, nasal aspirate, breath, urine, semen, saliva, peritoneal washings, cystic fluid, meningeal fluid, amniotic fluid, glandular fluid, lymph fluid, cytologic fluid, ascites, pleural fluid, nipple aspirate, bronchial aspirate, bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (broncho-alveolar lavage), pleura, thyroid, breast, pancreas and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

Further, it should be realized that a biological sample can be derived by taking biological samples from a number of individuals and pooling them or pooling an aliquot of each individual's biological sample. The pooled sample can be treated as a sample from a single individual and if the presence of a condition or disease is established in the pooled sample, then each individual biological sample can be re-tested to determine which individual(s) have that condition or disease.

In one embodiment, the present invention provides a method for evaluating a sample from an individual with Down syndrome for a condition or disease, comprising: preparing a biomarker profile from a biological sample obtained from the individual, and determining the presence or absence of a biomarker signature indicative of the condition or disease, the biomarker profile comprising the level, abundance, or concentration of at least two biomarkers listed in Table 1 or Table 2. In one embodiment, the at least two biomarkers are selected from the group consisting of FGFR1, EGFR, ERBB3, ERBB4, PDGFR, NRP1, MMPI, B2-microglobulin (B2M), Cystatin C (CST3), Annexin II (ANXA2), Notch1, Notch3, TNF sR-1 (TNFRSFIA), TNF sR-2 (TNFRSF1B), CD30 (TNFRSF8), CD30 ligand (TNFSFS), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 and IgE. In one embodiment, the biological sample is a blood sample. In one embodiment of a method for evaluating a sample from an individual with Down syndrome for a condition or disease, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea. In another embodiment, the biological sample is a blood sample.

For purposes of this specification, the phrase “data attributed to a biological sample from an individual” is intended to mean that the data in some form derived from, or were generated using, the biological sample of the individual. The data may have been reformatted, revised, or mathematically altered to some degree after having been generated, such as by conversion from units in one measurement system to units in another measurement system; but, the data are understood to have been derived from, or were generated using, the biological sample.

As used herein, the terms “biomarker” and “marker” are used interchangeably to refer to a target molecule that indicates or is a sign of a normal or abnormal process in an individual or of a disease or other' condition in an individual. More specifically, a “marker” or “biomarker” is an anatomic, physiologic, biochemical, or' molecular parameter associated with the presence of a specific physiological state or process, whether normal or abnormal, and, if abnormal, whether chronic or acute. Biomarkers are detectable and measurable by a variety of methods including laboratory assays and medical imaging. When a biomarker is a protein, it is also possible to use the expression of the corresponding gene as a surrogate measure of the amount or presence or absence of the corresponding protein biomarker in a biological sample or methylation state of the gene encoding the biomarker or proteins that control expression of the biomarker.

As used herein, “biomarker value”, “value”, “biomarker level”, and “level” are used interchangeably to refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or' corresponding to the biomarker in the biological sample. The exact nature of the “value” or “level” depends on the specific design and components of the particular analytical method employed to detect the biomarker.

As used herein, “reference value” refers to a pre-determined value of the level or concentration of a biomarker ascertained from a known sample. For instance, the reference value can reflect the level or concentration of a biomarker in a sample obtained from a typical individual (i.e., an individual without Down syndrome). In other embodiments, the reference value can reflect the level or concentration of a biomarker in a sample obtained from an individual at a particular stage in the condition or disease .e., exhibiting specific clinical criteria) or from an individual with a particular form of the disease. In still other embodiments, the reference value can reflect the level or concentration of a biomarker in an initial or baseline sample (i.e., pre-treatment sample) from an individual. A reference value can also be a known amount of a biomarker. Such a known amount of a biomarker may correlate with an average level of the biomarker from a population of typical individuals, a population of individuals with Down syndrome with a particular disease spectrum, a population of typical individuals with a particular disease spectrum, or population of individuals with Down syndrome at a particular stage of a condition or disease. In another embodiment, the reference value can be a range of values, which, for instance, can represent a mean plus or minus a standard deviation or confidence interval. A range of reference values can also refer to individual reference values for a particular biomarker across various disease outcomes.

When a biomarker indicates or is a sign of an abnormal process or a disease or other condition in an individual, that biomarker is generally described as being either over-expressed or under-expressed as compared to an expression level or value of the biomarker that indicates or is a sign of a normal process or an absence of a disease or other condition in an individual. “Up-regulation”, “up-regulated”, “over-expression”, “over-expressed”, and any variations thereof are used interchangeably to refer to a value or level of a biomarker in a biological sample that is greater than a value or level (or range of values or levels) of the biomarker that is typically detected in similar biological samples from healthy or normal individuals. The terms may also refer to a value or level of a biomarker in a biological sample that is greater than a value or level (or range of values or levels) of the biomarker that may be detected at a different stage of a particular disease.

“Down-regulation”, “down-regulated”, “under-expression”, “under-expressed”, and any variations thereof are used interchangeably to refer to a value or level of a biomarker in a biological sample that is less than a value or level (or range of values or levels) of the biomarker that is typically detected in similar biological samples from healthy or normal individuals. The terms may also refer to a value or level of a biomarker in a biological sample that is less than a value or level (or range of values or levels) of the biomarker that may be detected at a different stage of a particular disease.

Further, a biomarker that is either over-expressed or under-expressed can also be referred to as being “differentially expressed” or as having a “differential level” or “differential value” as compared to a “normal” expression level or value of the biomarker that indicates or is a sign of a normal process or an absence of a disease or other condition in an individual. Thus, “differential expression” of a biomarker can also be referred to as a variation from a “normal” expression level of the biomarker.

The term “differential gene expression” and “differential expression” are used interchangeably to refer to a gene (or its corresponding protein expression product) whose expression is activated to a higher or lower level in a subject suffering from a specific disease, relative to its expression in a normal or control subject. The terms also include genes (or the corresponding protein expression products) whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a variety of changes including mRNA levels, surface expression, secretion or other partitioning of a polypeptide. Differential gene expression may include a comparison of expression between two or more genes or their gene products; or a comparison of the ratios of the expression between two or more genes or their gene products; or even a comparison of two differently processed products of the same gene, which differ between normal subjects and subjects suffering from a disease; or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

In one embodiment, the method comprises measuring one or more biomarkers selected from the biomarkers listed in Table 1 or Table 2. In another embodiment, the one or more biomarkers is fibroblast growth factor receptor, epidermal growth factor receptor, platelet-derived growth factor receptor, neuropilin, matrix metalloproteinase, B2-microglobulin, Cystatin C, Annexin II, Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin. In another embodiment, the one or more biomarkers is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSFIB), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1 C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE.

In one embodiment, the number of biomarkers useful for a biomarker subset or panel is based on the sensitivity and specificity value for the particular combination of biomarker values. The terms “sensitivity” and “specificity” are used herein with respect to the ability to correctly classify an individual, based on one or more biomarker values detected in their biological sample, as having, for example, a condition or disease more prevalent in individuals with Down syndrome than in typical individuals or not having a condition or disease more prevalent in individuals with Down syndrome than in typical individuals. “Sensitivity” indicates the performance of the biomarker(s) with respect to correctly classifying individuals with Down syndrome that have a condition or disease more prevalent in individuals with Down syndrome than in typical individuals. “Specificity” indicates the performance of the biomarker(s) with respect to correctly classifying individuals with Down syndrome who do not have a condition or disease more prevalent in individuals with Down syndrome than in typical individuals.

As used herein, the term “individual” refers to a test subject or patient. The individual can be a mammal or a non-mammal, In various embodiments, the individual is a mammal. A mammalian individual can be a human or non-human. In various embodiments, the individual is a human. A healthy or normal individual is an individual in which the disease or condition of interest is not detectable by conventional diagnostic methods.

As used herein, the terms “condition”, “disease” or “disease state” includes all disease which result or could potentially cause a change, for example, in the functional genome, transcriptome, proteome, metabolome, epigenome, or microbiome of a subject afflicted with said disease. Examples of diseases include metabolic diseases (e.g., obesity, cachexia, diabetes, anorexia, etc.), cardiovascular diseases (e.g., atherosclerosis, ischemia/reperfusion, hypertension, restenosis, arterial inflammation, etc.), immunological disorders (e.g., chronic inflammatory diseases and disorders, such as Crohn's disease, reactive arthritis, including Lyme disease, insulin-dependent diabetes, organ-specific autoimmunity, including multiple sclerosis, Hashimoto's thyroiditis and Grave's disease, contact dermatitis, psoriasis, graft rejection, graft versus host disease, sarcoidosis, atopic conditions, such as asthma and allergy, including allergic rhinitis, gastrointestinal allergies, including food allergies, eosinophilia, conjunctivitis, glomerular nephritis, certain pathogen susceptibilities such as helminthic (e.g., leishmaniasis) and certain viral infections, including HIV, and bacterial infections, including tuberculosis and lepromatous leprosy, etc.), nervous system disorders (e.g., neuropathies, Alzheimer disease, Parkinson's disease, Huntington's disease, amyotrophic lateral sclerosis, motor neuron disease, traumatic nerve injury, multiple sclerosis, acute disseminated encephalomyelitis, acute necrotizing hemorrhagic leukoencephalitis, dysmyelination disease, mitochondrial disease, migrainous disorder, bacterial infection, fungal infection, stroke, aging, dementia, peripheral nervous system diseases and mental disorders such as depression and schizophrenia, etc.), oncological disorders (e.g., leukemia, brain cancer, pancreatic cancer, prostate cancer, liver cancer, stomach cancer, colon cancer, throat cancer, breast cancer, ovarian cancer, skin cancer, melanoma, etc.). The term also includes disorders which result from oxidative stress. Individuals having disease or in a disease state can be individuals with Down syndrome or typical individuals. In one embodiment, individuals with Down syndrome have a condition and/or disease that is more prevalent in individuals with Down syndrome than in typical individuals. For example, such conditions more prevalent in individuals with Down syndrome can include Alzheimer's disease, diabetes, autism and/or autism spectrum disorders, congenital heart defects, celiac disease, thyroid dysfunction, autoimmune disorders, vision problems, hearing problems, intestinal atresia, and sleep apnea. In another embodiment, typical individuals have a condition and/or disease that is more prevalent in typical individuals than in individuals with Down syndrome. For example, such conditions more prevalent in typical individuals can include various forms of heart disease, cancer, stroke, coronary heart disease, atherosclerosis, hypertension, angiopathies, and diabetic retinopathies. As will be understood by the skilled equipped with the instant disclosure, the biomarkers described in tables 1 and 2 can be used to not only identify proteins and pathways associated with conditions more prevalent in individuals with Down syndrome, but can also be used to design modalities for the treatment of conditions prevalent in typical individuals but typically absent or rarely occurring in individuals with Down syndrome.

As used herein, the term “disease spectrum” refers to the set of conditions or diseases associated with a given individual. In one embodiment, the individual is an individual with Down syndrome. In another embodiment, the individual is a typical individual. For example, one individual with Down syndrome may have Alzheimer's, thyroid dysfunction, infantile spasms and autism, while another individual with Down syndrome may have Alzheimer's, Type I Diabetes, leukemia and congenital heart defects. As another example, one typical individual may have heart disease and hypertension, while another typical individual may have cancer, diabetic retinopathies and atherosclerosis.

As used herein, the term “disease prevalence” or variations of this term refers to the number of all new and old cases of a disease or occurrences of an event during a particular period. Prevalence is expressed as a ratio in which the number of events is the numerator and the population at risk is the denominator.

As used herein, the terms “target”, “target molecule”, and “analyte” are used interchangeably herein to refer to any molecule of interest that may be present in a biological sample. A “molecule of interest” includes any minor variation of a particular molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule”, “target”, or “analyte” is a set of copies of one type or species of molecule or multi-molecular structure. “Target molecules”, “targets”, and “analytes” refer to more than one such set of molecules. Exemplary target molecules include proteins, polypeptides, nucleic acids, carbohydrates, lipids, polysaccharides, glycoproteins, hormones, receptors, antigens, antibodies, affybodies, autoantibodies, antibody mimics, viruses, pathogens, toxic substances, substrates, metabolites, transition state analogs, cofactors, inhibitors, drugs, dyes, nutrients, growth factors, cells, tissues, and any fragment or portion of any of the foregoing.

As used herein, the terms “polypeptide,” “peptide,” and “protein” are used interchangeably to refer to polymers of amino acids of any length, The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can be single chains or associated chains. Also included within the definition are preproteins and intact mature proteins; peptides or polypeptides derived from a mature protein; fragments of a protein; splice variants; recombinant forms of a protein; protein variants with amino acid modifications, deletions, or substitutions; digests; and post-translational modifications, such as glycosylation, acetylation, phosphorylation, and the like.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA, It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the terms “diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual. For example, the diagnosis of a condition or disease in an individual with Down syndrome includes distinguishing individuals with Down syndrome who have this condition or disease from individuals with Down syndrome who do not. It further includes distinguishing the severity of the same condition or disease between two individuals with Down syndrome who have the same condition or disease, In one embodiment, the present invention provides a method of confirming or refuting a diagnosis of a condition or disease in an individual with Down syndrome comprising; (a) measuring the level of at least one biomarker and/or the ratio of at least two biomarkers listed in Table 1 or Table 2 in a biological sample obtained from said individual; and (b) comparing the measured level or ratio to a reference value or range of reference values, wherein the diagnosis of the condition or disease in said individual is confirmed or refuted based on a change in the level of the at least one biomarker or the ratio of at least two biomarkers. In one embodiment of the method of confirming or refuting a diagnosis of a condition or disease in an individual with Down syndrome, the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1 S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE. In another embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

As used herein, the terms “prognose”, “prognosing”, “prognosis”, and variations thereof refer to the prediction of a future course of a disease or condition in an individual who has the disease or condition (e.g., predicting patient survival), and such terms encompass the evaluation of disease response after the administration of a treatment or therapy to the individual. In one embodiment, the present invention provides a method of monitoring treatment of a condition or disease in an individual with Down syndrome in need thereof comprising: (a) obtaining at least one initial biological sample from the individual at an initial time point, wherein the initial time point is prior to the start of a therapeutic intervention protocol for the condition or disease; (b) obtaining at least one subsequent biological sample from the individual at a subsequent time point, wherein the subsequent time point is after the start of the therapeutic intervention protocol; (c) measuring the level of at least one biomarker or panel of biomarkers listed in Table 1 or Table 2 in the initial and subsequent biological samples; and (d) comparing the level of the at least one biomarker or panel of biomarkers in the at least one initial biological sample to the level of the at least one biomarker or panel of biomarkers in the at least one subsequent biological sample, wherein a change in the level of the at least one biomarker or panel of biomarkers is indicative of the efficacy of the therapeutic intervention protocol, In one embodiment, the method of monitoring treatment of a condition or disease in an individual with Down syndrome in need thereof further comprises modifying or changing the therapeutic intervention protocol based on the level of one or more biomarkers. In another embodiment, the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSFS), OPG (TNFRSF11B), TAJ, (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE. In another embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

As used herein, the terms “evaluate”, “evaluating”, “evaluation”, and variations thereof encompass both “diagnose” and “prognose” and also encompass determinations or predictions about the future course of a disease or condition in an individual who does not have the disease as well as determinations or predictions regarding the likelihood that a disease or condition will recur in an individual who apparently has been cured of the disease. The term “evaluate” also encompasses assessing an individual's response to a therapy, such as, for example, predicting whether an individual is likely to respond favorably to a therapeutic agent or is unlikely to respond to a therapeutic agent (or will experience toxic or other undesirable side effects, for example), selecting a therapeutic agent for administration to an individual, or monitoring or determining an individual's response to a therapy that has been administered to the individual. Thus, “evaluating” a condition or disease in an individual with Down syndrome can include, for example, any of the following: prognosing the future course of the condition or disease in the individual with Down syndrome; predicting the recurrence of the condition or disease in the individual with Down syndrome who apparently has been cured of the condition or disease; or determining or predicting the individual with Down syndrome's response to a treatment for the condition or disease, or selecting a treatment for the condition or disease to administer to the individual with Down syndrome based upon a determination of the biomarker values derived from the individual with Down syndrome's biological sample.

Any of the following examples may be referred to as either “diagnosing” or “evaluating” a condition or disease associated with an individual with Down syndrome or a typical individual: initially detecting the presence or absence of the condition or disease; determining a specific stage, type or sub-type, or other classification or characteristic of the condition or disease; or detecting/monitoring progression of the condition or disease, remission, or recurrence. In one embodiment, the present invention provides a method of evaluating a condition or disease prevalent in an individual with Down syndrome compared to a typical individual, comprising: (a) measuring the level of at least one biomarker and/or the ratio of at least two biomarkers listed in Table 1 or Table 2 in a biological sample obtained from the individual with Down syndrome; and (b) comparing the measured level or ratio to a reference value or range of reference values, wherein the reference is one or more typical individuals, and wherein a change in the level of at least one biomarker or the ratio of at least two biomarkers is indicative of the condition or disease prevalent in the individual with Down syndrome. In another embodiment, the present invention provides a method of evaluating a condition or disease prevalent in a typical individual but rare in an individual with Down syndrome, comprising: (a) measuring the level of at least one biomarker and/or the ratio of at least two biomarkers in a biological sample obtained from the typical individual; and (b) comparing the measured level or ratio to a reference value or range of reference values, wherein the reference is one or more individuals with Down syndrome, and wherein a change in the level of at least one biomarker or the ratio of at least two biomarkers is indicative of the condition or disease prevalent in the typical individual. In one embodiment, at least one biomarker is selected from Table 1 or Table 2. In another embodiment, the at least one biomarker is fibroblast growth factor receptor, epidermal growth factor receptor, platelet-derived growth factor receptor, neuropilin, matrix metalloproteinase, B2-microglobulin, Cystatin C, Annexin II, Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin. In another embodiment, the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSFIA), TNF sR-2 (TNFRSF1B), CD30 (TNFRSFB), CD30 ligand (TNFSF8), OPG (TNFRSF11B), TAJ (TNFRSF19), DR3 (TNFRSF25), 4-1BB (TNFRSF9)_{;} 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3)_{;} IGFBP6 or IgE.

As used herein, “additional biomedical information” refers to one or more evaluations of an individual with Down syndrome or a typical individual, other than using any of the biomarkers described herein, that are associated with risk for a condition or disease. “Additional biomedical information” includes any of the following: physical descriptors of an individual, the height and/or weight of an individual, the gender of an individual, the ethnicity of an individual, smoking history, occupational history, exposure to known carcinogens (e.g., exposure to any of asbestos, radon gas, chemicals, smoke from fires, and air pollution, which can include emissions from stationary or mobile sources such as industrial/factory or auto/marine/aircraft emissions), exposure to second-hand smoke, family history of disease, and the like. Additional biomedical information can be obtained from an individual using routine techniques known in the art, such as from the individual themselves by use of a routine patient questionnaire or health history questionnaire, etc., or from a medical practitioner, etc. Alternately, additional biomedical information can be obtained from routine imaging techniques, including CT imaging (e.g., low-dose CT imaging) and X-ray. Testing of biomarker levels in combination with an evaluation of any additional biomedical information may, for example, improve sensitivity, specificity and/or AUC for detecting a condition or disease as compared to biomarker testing alone or evaluating any particular item of additional biomedical information alone (e.g., CT imaging alone). Additional biomedical information can be obtained from electronic medical records.

As used herein, the term “kit” or “assay kit” (e.g., articles of manufacture) refers to an assembly of useful compounds and other means like solid support plates or test strips for detecting one or more biomarkers in a biological sample collected from one or more individuals. Components such as buffers, controls, and the like, known to those of ordinary skill in art, may be included in such test kits. The relative amounts of the various reagents can be varied, to provide for concentrations in solution of the reagents that substantially optimize the sensitivity of the assay. Particularly, the reagents can be provided as dry powders, usually lyophilized, which on dissolution will provide for a reagent solution having the appropriate concentrations for combining with a sample. The present kit may further include instructions for carrying out one or more methods of the present invention, including instructions for using any device and/or composition of the present invention that is included with the kit.

In one embodiment, the present invention provides a biomarker kit comprising reagents for measuring one or more biomarkers listed in Table 1 or Table 2. In one embodiment, the one or more biomarkers is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1 IMPDH2, C1CQBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE. In another embodiment, the kit further comprises a set of reference values to which the levels of the one or more biomarkers can be compared. In another embodiment, the reagents are adapted for measuring biomarkers in a blood sample. In another embodiment, the kit further comprises instructions for measuring said one or more biomarkers for diagnosing, evaluating level of severity, or monitoring progression of a condition or disease in an individual with Down syndrome. In another embodiment, the kit further comprises instructions for measuring said one or more biomarkers for monitoring the efficacy of a therapeutic intervention in an individual with Down syndrome having a condition or disease. In another embodiment, the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

The term “area under the curve” or “AUC” refers to the area under the curve of a receiver' operating characteristic (ROC) curve, both of which are well known in the art. AUC measures are useful for comparing the accuracy of a classifier' across the complete data range. Classifiers with a greater AUC have a greater capacity to classify unknowns correctly between two groups of interest (e.g., diseased samples and normal or control samples). ROC curves are useful for plotting the performance of a particular feature (e.g., any of the biomarkers described herein and/or any item of additional biomedical information) in distinguishing between two populations (e.g., cases having a disease and controls without disease). Typically, the feature data across the entire population (e.g., the cases and controls) are sorted in ascending order based on the value of a single feature. Then, for each value for that feature, the true positive and false positive rates for the data are calculated, The true positive rate is determined by counting the number of cases above the value for that feature and then dividing by the total number of cases, The false positive rate is determined by counting the number of controls above the value for that feature and then dividing by the total number of controls, Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve. These combinations of features may comprise a test. The ROC curve is the plot of the true positive rate (sensitivity) of a test against the false positive rate (1-specificity) of the test.

As used herein, “detecting” or “determining” with respect to a biomarker value includes the use of both the instrument required to observe and record a signal corresponding to a biomarker value and the material/s required to generate that signal. In various embodiments, the biomarker value is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

As used herein, the term “healthy” refers to individuals, whether they have Down syndrome or are typical, when a given condition and/or disease is not present compared to another individual where the condition and/or disease is present.

As used herein, the term “therapeutic” means an agent utilized to discourage, combat, ameliorate, prevent or improve an unwanted condition, disease or symptom of a patient. In one embodiment, the present invention provides a method of evaluating the therapeutic efficacy of a therapeutic intervention for treating a condition or disease in an individual with Down syndrome comprising: (a) obtaining at least one initial biological sample from the individual at an initial time point, wherein the initial time point is prior to the administration of the therapeutic intervention; (b) obtaining at least one subsequent biological sample from the individual at a subsequent time point, wherein the subsequent time point is after the administration of the therapeutic intervention; (c) measuring the level of at least one biomarker listed in Table 1 or Table 2 in the initial and subsequent biological samples; and (d) comparing the level of at least one biomarker in at least one initial biological sample to the level of at least one biomarker in at least one subsequent biological sample, wherein a change in the level of at least one biomarker is indicative of the efficacy of the therapeutic intervention as a treatment for the condition or disease in the individual with Down syndrome. In another embodiment, the at least one biomarker is a peptide, polypeptide, protein, polynucleotide, transcript, small molecule or microbiome profile. In another embodiment, the at least one biomarker is a surrogate marker. In another embodiment, the initial and subsequent biological samples are selected from the group consisting of saliva, tears, buccal swab, nasal epithelium, skin, plasma, urine, blood and stool. In another embodiment, the initial and subsequent biological samples are blood. In another embodiment, the at least one biomarker is selected from Table 1 or Table 2. In another embodiment, the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSFB), OPG (TNFRSF11B), TAJ (TNFRSF19), DR3 (TNFRSF25), 4-1BB (TNFRSF9), 4-IBB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE.

In another embodiment, the at least one biomarker functions in a pathway selected from the group consisting of acute phase response signaling, the complement pathway and the prothrombin pathway. In one embodiment, the at least one biomarker functions in the acute phase response signaling. In another embodiment, the at least one biomarker is TNF sR-I (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9) or 4-1BB ligand (TNFSF9). In one embodiment, the at least one biomarker functions in the complement pathway. In another embodiment, the at least one biomarker is C1C1BP, C1R, C1S, C3, C6, C7, CFH or CFP. In one embodiment, the at least one biomarker functions in the prothrombin pathway. In another embodiment, the at least one biomarker is SerpinC1 (antithrombin III), MBL2 or F10 (Factor Xa),

In another embodiment, the therapeutic intervention is a compound or biologic selected from a compound or biologic library. In another embodiment, the condition or disease comprises Alzheimer's, cognitive impairment, epilepsy, leukemia, diabetes, autism, congenital head defects, celiac disease, thyroid dysfunction, autoimmune disorders, vision problems, hearing problems, intestinal atresia and/or sleep apnea.

As the term is used herein, “treating” and “treatment” refers to an action which results in an improvement in a disease or disorder, for example, beneficial or desired clinical results. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease.

As used herein, the terms “typicals” or “typical individual” refer to individuals not affected by Down syndrome.

Experiments encompassed by the present invention seek to significantly accelerate research on Down syndrome to the point of rapidly making it one of the most well understood medical conditions. One goal of the present invention is to define how trisomy 21 causes a novel disease spectrum. Another goal of the present invention is to apply the knowledge gained from research for the development and delivery of novel diagnostic and therapeutic tools that will not only benefit those with trisomy 21, but also millions of typical individuals.

Certain conditions and/or diseases are more common among people with Down syndrome compared to unaffected individuals. For example, individuals with Down syndrome have higher incidence of Alzheimer's, diabetes, autism, congenital heart defects, celiac disease, thyroid dysfunction, autoimmune disorders, vision problems, hearing problems, intestinal atresia and sleep apnea. Conversely, it has also been observed that individuals with Down syndrome can also have reduced incidence of other conditions and/or diseases that are more prevalent in typical individuals such as heart disease, cancer, stroke, coronary artery disease, atherosclerosis, hypertension and angiopathies (e.g. diabetic retinopathies). Trisomy 21 is required but not sufficient to cause the various conditions and/or diseases associated with Down syndrome. In addition, the set of conditions and/or diseases associated with a given individual with Down syndrome may be different from the set of conditions and/or diseases associated with another individual with Down syndrome. Thus understanding the causal factors of these conditions and/or diseases would inform development of diagnostics and therapeutics not only for individuals with Down syndrome but for the general population also.

To determine the auxiliary causal factors of various conditions associated with Down syndrome, data can be gathered from individuals with Down syndrome plus control subjects, some of which may be close relatives of the individuals with Down syndrome to capture various layers of information including electronic medical records, stem cells, genome, epigenome, transcriptome, proteome, microbiome, metabolome, functional genome and bloodworks,

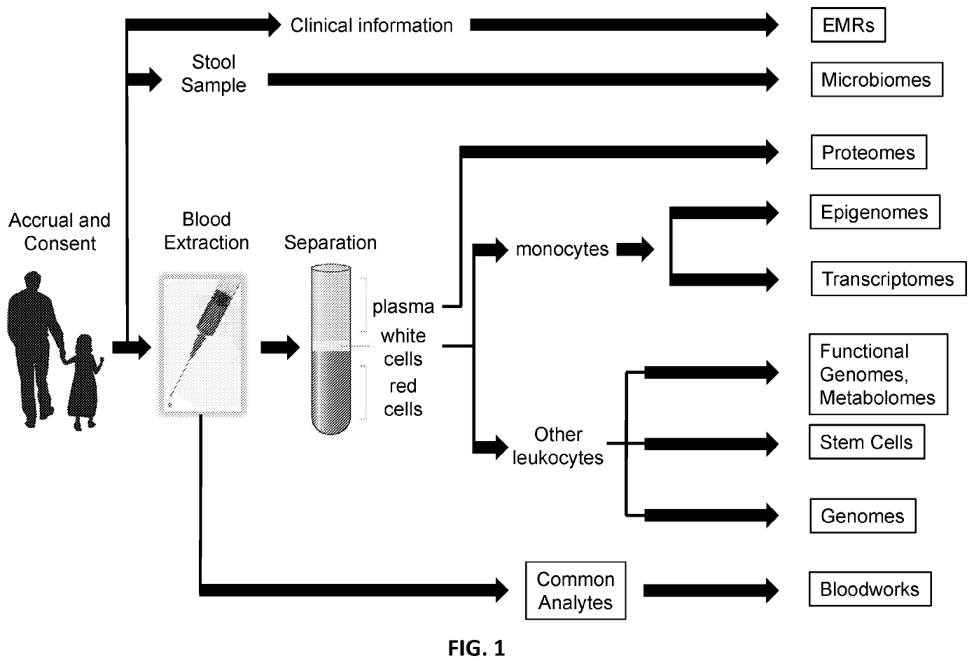

Blood can be extracted from individuals with Down syndrome and control subjects and separated into plasma, white blood cells and red blood cells. From plasma a proteome of >3700 proteins can be assessed. The white blood cells can be separated into monocytes and all other leukocytes. Epigenomes and transcriptomes can be analyzed in the monocytes. Lymphoblastoids can be used for functional genomics and metabolomics analyses. Induced pluripotent stem cells (iPSCs) can be produced from lymphocytes, and the iPSCs can be used to derive neurons, pancreas cells, bone cells, etc. Whole genome sequencing can be done on leukocytes. Bloodworks may involve using mass cytometry or CyTOF (Cytometry by Time-Of-Flight) to detect biomarkers (FIG. 1).

Stool samples can be collected for microbiome analysis (FIG. 1).

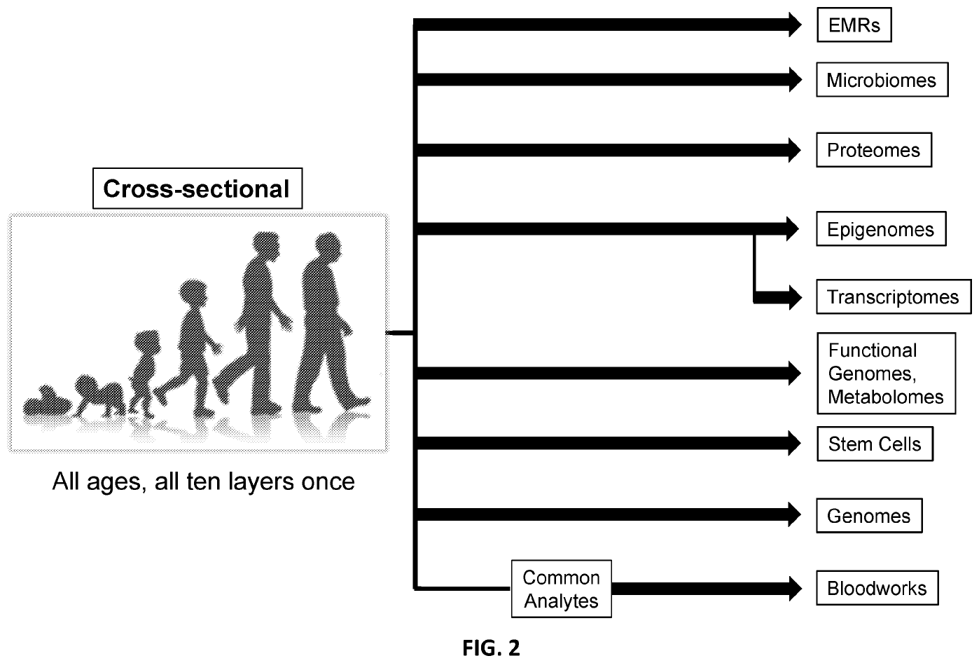

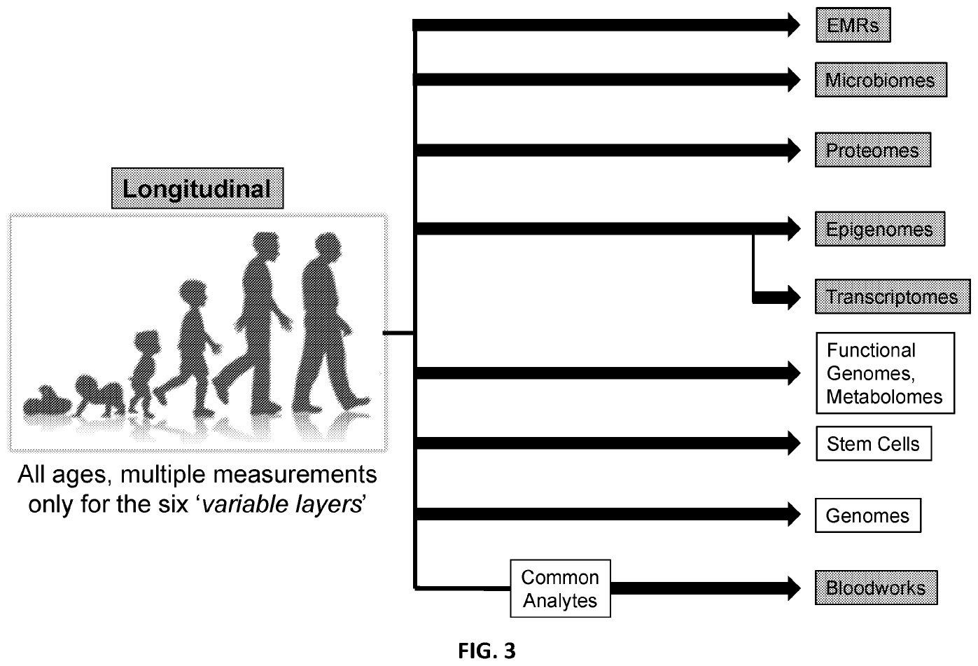

Data gathering may be cross-sectional, including Down syndrome and control individuals of all ages and obtaining data in all ten layers once (FIG. 2).

Data gathering may also be longitudinal, including Down syndrome and control individuals of all ages and obtaining multiple measurements for the six variable layers: EMRs, microtomes, proteomes, epigenomes, transcriptomes and bloodworks (FIG. 3).

The analysis of the ten layers yields multi-dimensional datasets that can be used to discover the molecular basis of a given disease. For example, EMRs may indicate extreme thirst, frequent urination, drowsiness or lethargy, increased appetite, sudden weight loss, sudden vision changes, heavy or labored breathing, stupor or unconsciousness. The analysis of genomes shows an increased frequency of certain HLA alleles. The analysis of transcriptomes shows low RNA levels of GLUT genes encoding cellular glucose transporters. The analysis of proteomes shows low insulin in plasma. The analysis of bloodworks show high glucose in plasma and a strong inflammatory signature (IFNγ, CD8+). A researcher may ask if there is a cause-effect relationship between specific HLA alleles, insulin production, cellular glucose uptake, an inflammatory signature and the levels of glucose in blood.

One hypothesis would be that certain HLA alleles cause an autoimmune response against insulin-producing cells, which lead to the loss of insulin production and systemic decrease in cellular glucose uptake. Thus high glucose is present in the blood, leading to exhibition of the observed symptoms in the subject.

Diagnostic opportunities for this example include genotyping for certain HLA alleles and monitoring glucose and insulin levels. Therapeutic opportunities for this example include insulin administration, diet control, blocking of the autoimmune response, stem cell therapy and beta cell transplant.

Several hypotheses can be proposed to explain the different disease spectra observed in different individuals with Down syndrome.

The Sensitized Background hypothesis posits that the different disease spectrum is caused by rare DNA variants (i.e. alleles) whose effect is greater in the genetic background of trisomy 21, These rare alleles may also be acting in the typical population, albeit with lower penetrance. The ability to identify DNA elements that protect from or predispose to various medical conditions or disease varies with the genetic background. The main datasets for this hypothesis include genuine, transcriptome and EMRs.

The 2 within 3 hypothesis posits that the inter-individual variation in conditions is due to the fact that some individuals have lost one functional copy of the gene whose trisomy drives the condition. Therefore, they do not present that particular condition. These individuals have 2 functional copies of the gene that causes a condition, but 3 copies of all other genes on chromosome 21. The main datasets for this hypothesis include genome, transcriptome and EMRs.

The Differential Microbiome Hypothesis posits that individuals with Down syndrome harbor a microbiome that is significantly different from that found in typical individuals. Differences in the microbiome may contribute to some aspects of the syndrome, such as chronic skin disorders and infections, immune dysfunction, poor gain weight, malabsorption, constipation and Hirschsprung's disease. If correct, the differential microbiome could enable diagnostic and therapeutic opportunities. The main datasets for this hypothesis include microbiome and EMRs.

Other hypotheses can be proposed by one skilled in the art.

### Acute Phase Response

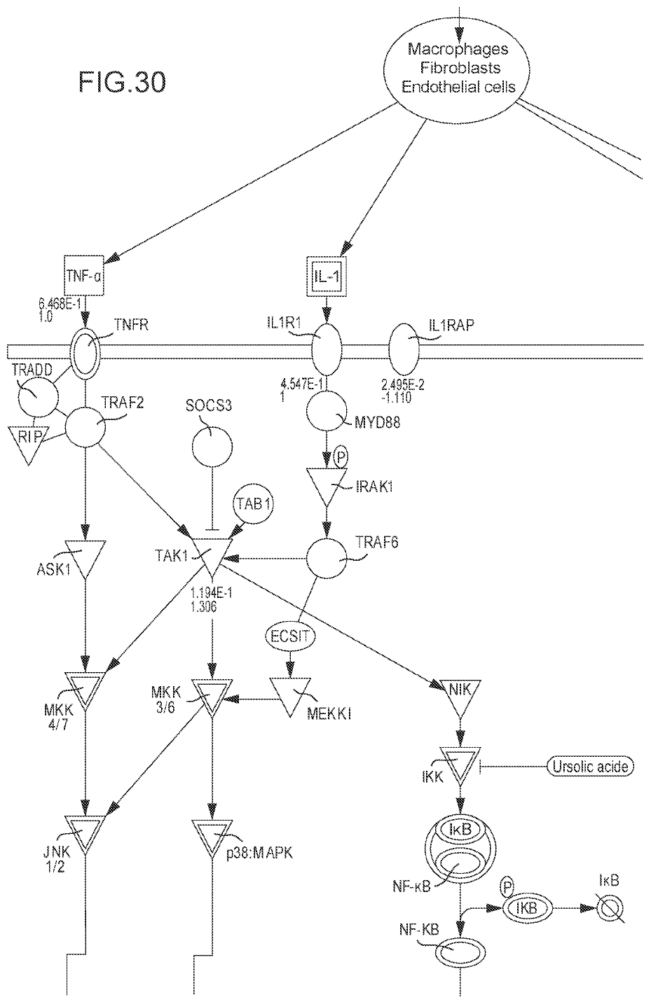

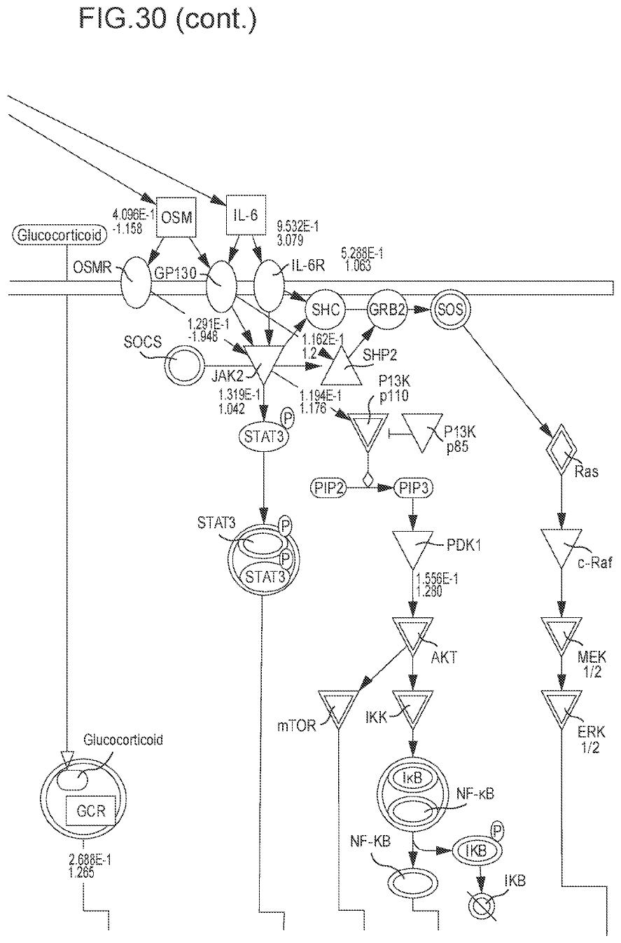

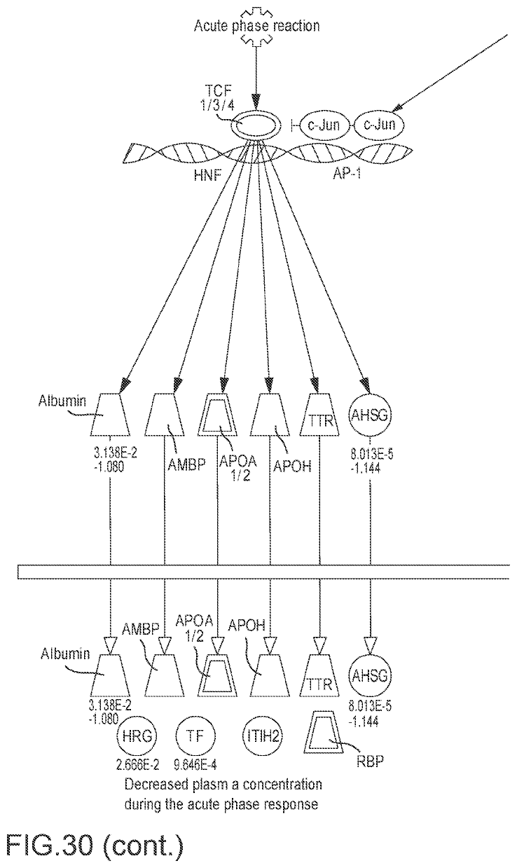

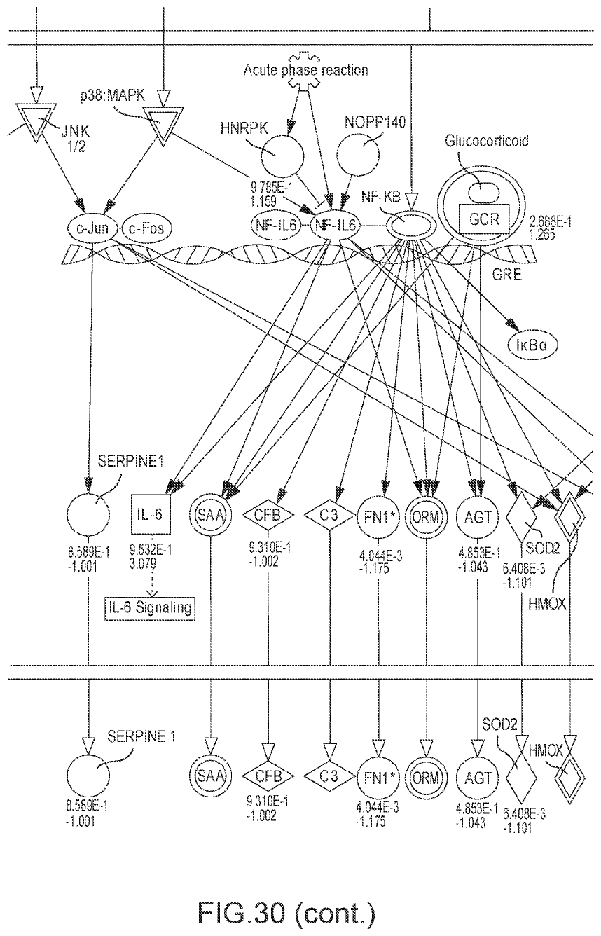

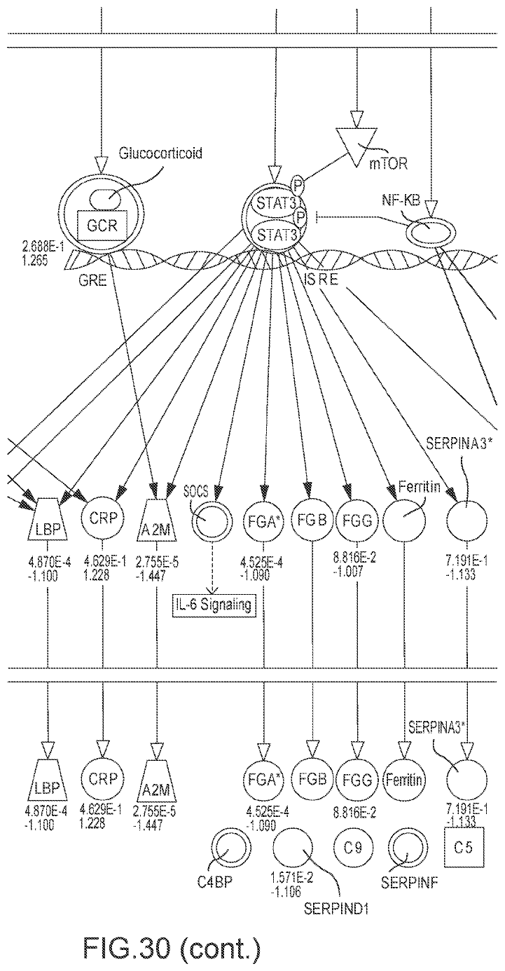

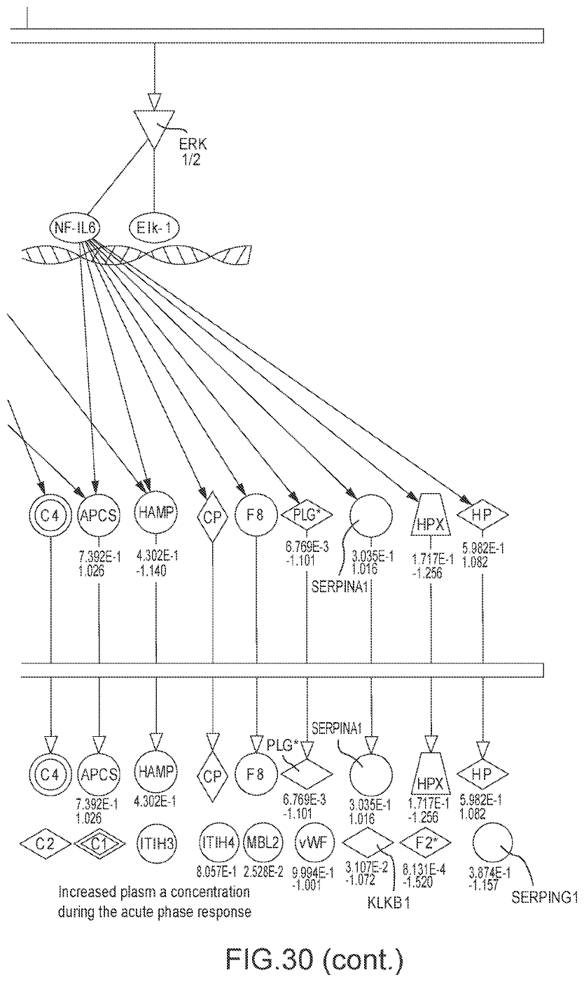

The acute phase response signaling pathway is illustrated in FIG. 30. A local inflammatory response can be triggered by infection, tissue injury, trauma or surgery, neoplastic growth or immunological disorders. In the local inflammatory response, the major pro-inflammatory cytokines interleukin-1 (IL-1), interleukin-6 (IL-6) and TNF-α are released and neutrophils, monocytes, macrophages and the vascular system are activated. These responses in turn are associated with production of more cytokines and other inflammatory mediators which diffuse to the extracellular fluid compartment and circulate in the blood.

The acute phase response is a prominent systemic reaction following the local inflammatory response and is characterized by reduction of growth hormone secretion, the induction of fever, anorexia, negative nitrogen balance and catabolism of muscle cells.

Furthermore a series of changes can be measured in the laboratory, such as: (1) a decrease of blood plasma low and high density lipoprotein-bound cholesterol and leukocyte numbers in blood, (2) increased values of adrenocorticotrophic hormone (ACTH) and glucocorticoids, (3) activation of the complement system and blood coagulation system, (4) decreased serum levels of calcium, zinc, iron, vitamin A and of a-tocopherol, and (5) a change in concentration of several plasma proteins, the acute phase proteins (APPs) largely due to an altered hepatic metabolism.

Within a few hours after infection the pattern of protein synthesis by the liver is drastically altered resulting in an increase of some blood proteins, the positive APPs. Hepatic mRNA upregulation of those APPs is associated with a decrease in synthesis of normal blood proteins, like transthyretin (TTR, formerly called prealbumin), retinol binding protein (RBP), cortisol binding globulin, transferrin and albumin, which represent the negative APPs.

The positive APPs include C-reactive protein (CRP), serum amyloid A (SAA) and haptoglobin (Hp) which are released by the hepatocytes after cytokine stimulation.

The acute phase response can affect many systems in an organism. Malnutrition and the anorectic effects of pro-inflammatory cytokines in the brain result in a negatively changed hepatic synthesis. Moreover, there is evidence that cytokines and their cognate receptors are present in the neuroendocrine system and brain. In addition, induction of the acute phase response and production of pro-inflammatory cytokines may directly affect the process of bone growth. Infection burdens often are associated with growth failure. This occurs because the infections may decrease food intake, impair nutrient absorption, cause direct nutrient losses, increase metabolic requirements and catabolic loss of nutrients and may impair transport of nutrients to target tissues.

The acute phase response with its changes in blood plasma composition is thought to be beneficial to the organism by preventing microbial growth and helping to restore homeostasis. Some APPs opsonize microorganisms and activate complement, while others scavenge cellular remnants and free radicals, or neutralize proteolytic enzymes.

Pro-inflammatory cytokines such as TNF-α, IL-1 and IL-6 activate hepatocytic receptors to allow synthesis of various APPs. Muscle protein functions as the major storage for the amino acids required for APP synthesis.

The positive APPs of man and domestic animals can generally be listed in three major groups: (1) ceruloplasmin and complement factor-3 (C3), with an increase of about 50%, (2) haptoglobin, fibrinogen, α-globulins with antiprotease-activity and lipopolysaccharide binding protein, with an increase of two-three fold, and (3) CRP and SAA, with a rapid increase of about 5-fold to 1000-fold.

CRP, a ring consisting of five 23,000 Da units (pentraxin), is the first described acute phase protein. It was discovered due to its binding to the C-polysaccharide of pneumococci. It binds directly to several microorganisms, degrading cells and cell remnants, and activates complement by the classical C1q pathway. It also acts as opsonin,

SAA is an apolipoprotein of high-density lipoprotein (apoSAA), It is thought to influence high-density lipoprotein-cholesterol transport, In tissues it attracts inflammatory cells and inhibits the respiratory burst of leukocytes and modulates the immune response, It has been observed to bind lipopolysaccharide, comparable to lipopolysaccharide binding protein (LBP). Several isotypes of SAA are found: types 1 and 2 represent positive APPs. Besides the acute phase SAAs, constitutive variants of SAA have been described: human SAA4 is normally present in serum; rabbit SAA3 is formed by synoviocytes, fibroblasts and macrophages, and is not a blood protein. The mammary gland is a well known source of an SAA3 variant occurring in colostrum and in mastitis milk that should have beneficial functions for the gut mucosa of the offspring.

Haptoglobin (Hp) strongly binds haemoglobin, has anti-inflammatory capabilities and binds to CD11b/CD18 integrins, which represent the major receptors on the cell membranes of leukocytes, Although representing a positive acute phase protein, its quantity may decrease on massive erythrolysis.

Ceruloplasmin (Cp) contains copper, has histaminase and ferroxidase activity, and scavenges Fe2+ and free radicals, while α2-macroglobulin (α2MG) binds proteolytic enzymes. The function of fibrinogen is clot-formation and C3 has complement function; acid glycoprotein (α1AGP), formerly called orosomucoid and which has been found not to react as a major (group C) acute phase protein in most domestic animal species except the cat, is reported to influence T-cell function and to bind steroids such as progesterone. The functions of α1-proteinase inhibitor which is also called α1-antitrypsin, a serine protease inhibitor or serpin, and α1-antichymotrypsin are inhibitors of leukocyte and lysosomal proteolytic enzymes.

Some disease states are associated with, or are causally related to APPs. The pathogenic role of fibrin in thrombosis is well known. Elevated serum values of CRP are associated with increased risk of human atherosclerosis. The causal relationship between the acute phase protein, SAA, and the extracellular deposition of amyloid fibrils has been proven but the mechanism of amyloid formation from the acute phase protein, however, remains to be elucidated.

During chronic infection, positive acute phase protein levels remain elevated in comparison to normal values, and can be used for diagnostic purposes. The significance of APPs as non-specific variables for monitoring inflammatory activity has been adopted in veterinary clinical chemistry. The acute phase signal/starvation situation obtained for an individual animal can be enhanced when the values of positive APPs (rapid and slow) are combined with those of rapid and slow negative APPs in an index (acute phase index (API) or nutritional and acute phase indicator, NAPI).

NAPI=(value of a rapid positive APP×value of a slow positive APP)/(value of rapid negative APP×value of a slow negative APP).

The index has been used as prognostic inflammatory and nutritional index (PINI) for human patients and as acute phase index (API) for cattle. In human patients a simple quotient of the values of CRP/TTR has already proven useful in monitoring bone fracture patients.

### Complement System

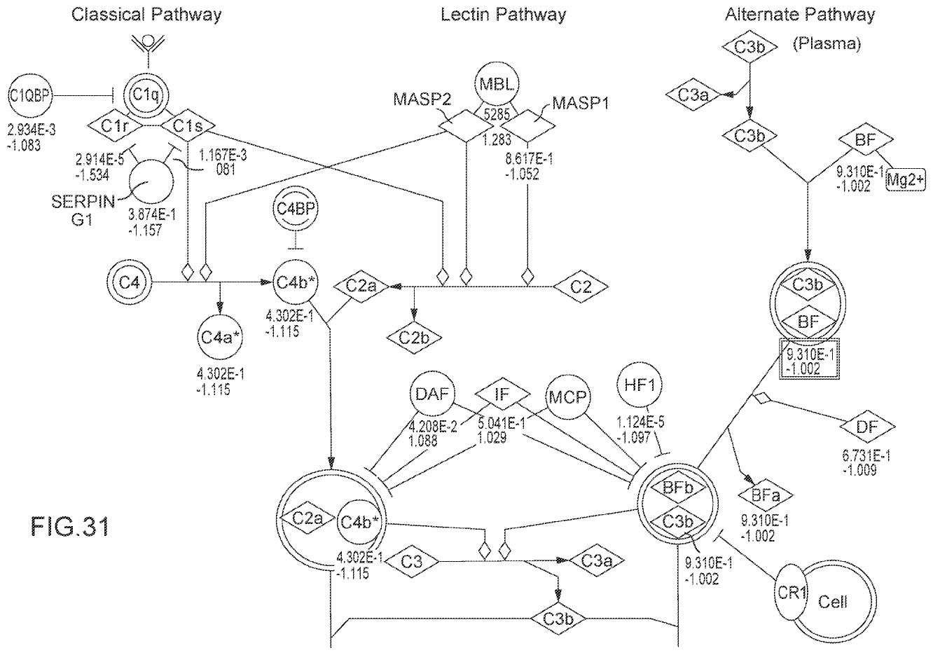

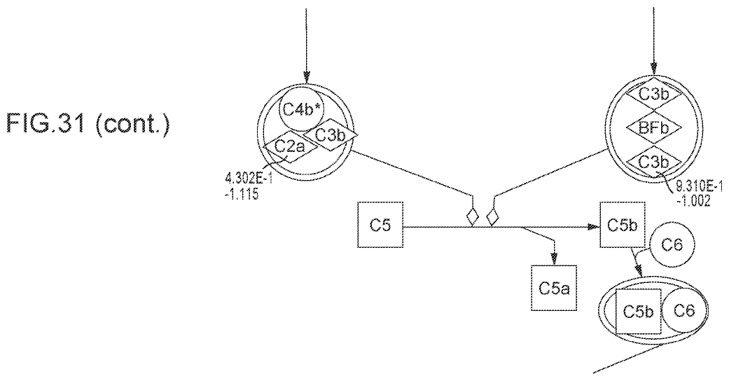

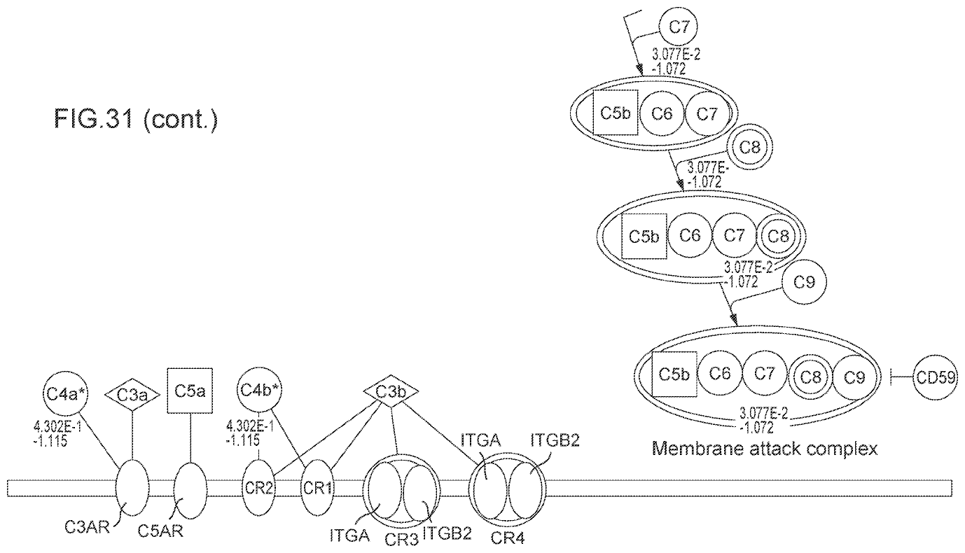

The complement system is illustrated in FIG. 31. The complement system is a part of the immune system that helps or complements the ability of antibodies and phagocytic cells to clear pathogens from an organism. It is part of the innate immune system, which is not adaptable and does not change over the course of an individual's lifetime. However, the complement pathway can be recruited and brought into action by the adaptive immune system.

The complement system comprises a number of small proteins found in the blood, in general synthesized by the liver, and normally circulating as inactive precursors (pro-proteins). When stimulated by one of several triggers, proteases in the system cleave specific proteins to release cytokines and initiate an amplifying cascade of further cleavages. The end result of this activation cascade is massive amplification of the response and activation of the cell-killing membrane attack complex. Over 30 proteins and protein fragments make up the complement system, including serum proteins, serosal proteins, and cell membrane receptors. They account for about 5% of the globulin fraction of blood serum and can serve as opsonins.

The complement pathway can: enhance phagocytosis of antigens (opsonization), attract macrophages and neutrophils (chemotaxis), rupture membranes of foreign cells (cell lysis) and/or cluster and bind pathogens together (agglutination). The proteins and glycoproteins that constitute the complement system are synthesized by hepatocytes. Significant amounts are also produced by tissue macrophages, blood monocytes, and epithelial cells of the genitourinal tract and gastrointestinal tract.

Three biochemical pathways activate the complement system: the classical complement pathway, the alternative complement pathway, and the lectin pathway. The three pathways of activation all generate homologous variants of the protease C3-convertase.

The classical complement pathway typically requires antigen-antibody complexes (immune complexes) for activation (specific immune response), whereas the alternative pathway can be activated by C3 hydrolysis, foreign material, pathogens, or damaged cells. The mannose-binding lectin pathway can be activated by C3 hydrolysis or antigens without the presence of antibodies (non-specific immune response). In all three pathways, C3-convertase cleaves and activates component C3, creating C3a and C3b, and causes a cascade of further cleavage and activation events. C3b binds to the surface of pathogens, leading to greater internalization by phagocytic cells by opsonization.

In the Alternative Pathway, C3b binds to Factor B. Factor D releases Factor Ba from Factor B bound to C3b. The complex of C3b(2)Bb is a protease which cleaves C5 into C5b and C5a. C5 convertase is also formed by the Classical Pathway when C3b binds C4b and C2a. C5a is an important chemotactic protein, helping recruit inflammatory cells. C3a is the precursor of an important cytokine (adipokine) named ASP and is usually rapidly cleaved by carboxypeptidase B. Both C3a and C5a have anaphylatoxin activity, directly triggering degranulation of mast cells as well as increasing vascular permeability and smooth muscle contraction. C5b initiates the membrane attack pathway, which results in the membrane attack complex (MAC), consisting of C5b, C6, C7, C8, and polymeric C9. MAC is the cytolytic end product of the complement cascade; it forms a transmembrane channel, which causes osmotic lysis of the target cell. Kupffer cells and other macrophage cell types help clear complement-coated pathogens.

The classical pathway is triggered by activation of the C1-complex. The C1-complex is composed of 1 molecule of C1q, 2 molecules of C1r and 2 molecules of C1s, or C1qr2s2. This occurs when C1q binds to IgM or IgG complexed with antigens. A single pentameric IgM can initiate the pathway, while several, ideally six, IgGs are needed. This also occurs when C1q binds directly to the surface of the pathogen. Such binding leads to conformational changes in the C1q molecule, which leads to the activation of two C1r molecules. C1r is a serine protease. They then cleave C1s (another serine protease). The C1r2s2 component now splits C4 and then C2, producing C4a, C4b, C2a, and C2b. C4b and C2a bind to form the classical pathway C3-convertase (C4b2a complex), which promotes cleavage of C3 into C3a and C3b; C3b later joins with C4b2a (the C3 convertase) to make C5 convertase (C4b2a3b complex). The inhibition of C1r and C1s is controlled by C1-inhibitor.

C3-convertase can be inhibited by Decay accelerating factor (DAF), which is bound to erythrocyte plasma membranes via a GPI anchor.

The alternative pathway is continuously activated at a low level as a result of spontaneous C3 hydrolysis due to the breakdown of the internal thioester bond (C3 is mildly unstable in aqueous environment), The alternative pathway does not rely on pathogen-binding antibodies like the other pathways. C3b that is generated from C3 by a C3 convertase enzyme complex in the fluid phase is rapidly inactivated by factor H and factor I, as is the C3b-dike C3 that is the product of spontaneous cleavage of the internal thioester. In contrast, when the internal thioester of C3 reacts with a hydroxyl or amino group of a molecule on the surface of a cell or pathogen, the C3b that is now covalently bound to the surface is protected from factor H-mediated inactivation. The surface-bound C3b may now bind factor B to form C3bB. This complex in the presence of factor D will be cleaved into Ba and Bb. Bb will remain associated with C3b to form C3bBb, which is the alternative pathway C3 convertase.

The C3bBb complex is stabilized by binding oligomers of factor P (Properdin). The stabilized 03 convertase, C3bBbP, then acts enzymatically to cleave much more C3, some of which becomes covalently attached to the same surface as C3b. This newly bound C3b recruits more B, D and P activity and greatly amplifies the complement activation. When complement is activated on a cell surface, the activation is limited by endogenous complement regulatory proteins, which include CD35, CD46, CD55 and CD59, depending on the cell. Pathogens, in general, don't have complement regulatory proteins (there are many exceptions, which reflect adaptation of microbial pathogens to vertebrate immune defenses). Thus, the alternative complement pathway is able to distinguish self from non-self on the basis of the surface expression of complement regulatory proteins. Host cells don't accumulate cell surface C3b (and the proteolytic fragment of C3b called iC3b) because this is prevented by the complement regulatory proteins, while foreign cells, pathogens and abnormal surfaces may be heavily decorated with C3b and iC3b. Accordingly, the alternative complement pathway is one element of innate immunity.

Once the alternative C3 convertase enzyme is formed on a pathogen or cell surface, it may bind covalently another C3b, to form C3bBbC3bP, the C5 convertase. This enzyme then cleaves C5 to C5a, a potent anaphylatoxin, and C5b. The C5b then recruits and assembles C6, C7, C8 and multiple C9 molecules to assemble the membrane attack complex. This creates a hole or pore in the membrane that can kill or damage the pathogen or cell.

The lectin pathway is homologous to the classical pathway, but with the opsonin, mannose-binding lectin (MBL), and ficolins, instead of C1q. This pathway is activated by binding of MBL to mannose residues on the pathogen surface, which activates the MBL-associated serine proteases, MASP-1, and MASP-2 (very similar to C1r and C1s, respectively), which can then split C4 into C4a and C4b and C2 into C2a and C2b. C4b and C2a then bind together to form the classical C3-convertase, as in the classical pathway.

Ficolins are homologous to MBL and function via MASP in a similar way. Several single-nucleotide polymorphisms have been described in M-ficolin in humans, with effect on ligand-binding ability and serum levels. Historically, the larger fragment of C2 was named C2a, but it is now referred as C2b. In invertebrates without an adaptive immune system, ficolins are expanded and their binding specificities diversified to compensate for the lack of pathogen-specific recognition molecules.

It is thought that the complement system might play a role in many diseases with an immune component, such as Barraquer-Simons Syndrome, asthma, lupus erythematosus, glomerulonephritis, various forms of arthritis, autoimmune heart disease, multiple sclerosis, inflammatory bowel disease, paroxysmal nocturnal hemoglobinuria, atypical hemolytic uremic syndrome and ischemia-reperfusion injuries, and rejection of transplanted organs.

The complement system is also becoming increasingly implicated in diseases of the central nervous system such as Alzheimer's disease and other neurodegenerative conditions such as spinal cord injuries.

Deficiencies of the terminal pathway predispose to both autoimmune disease and infections (particularly Neisseria meningitis, due to the role that the membrane attack complex (“MAC”) plays in attacking Gram-negative bacteria.

Mutations in the complement regulators factor H and membrane cofactor protein have been associated with atypical hemolytic uremic syndrome. Moreover, a common single nucleotide polymorphism in factor H (Y402H) has been associated with the common eye disease age-related macular degeneration. Polymorphisms of complement component 3, complement factor B, and complement factor I, as well as deletion of complement factor H-related 3 and complement factor H-related 1 also affect a person's risk of developing age-related macular degeneration. Both of these disorders are currently thought to be due to aberrant complement activation on the surface of host cells.

Mutations in the Cl inhibitor gene can cause hereditary angioedema, a genetic condition resulting from reduced regulation of bradykinin by C1-INH.

Mutations in the MAC components of complement, especially C8, are often implicated in recurrent Neisserial infection.

Diagnostic tools to measure complement activity include the total complement activity test.

### Intrinsic Prothrombin Activation Pathway

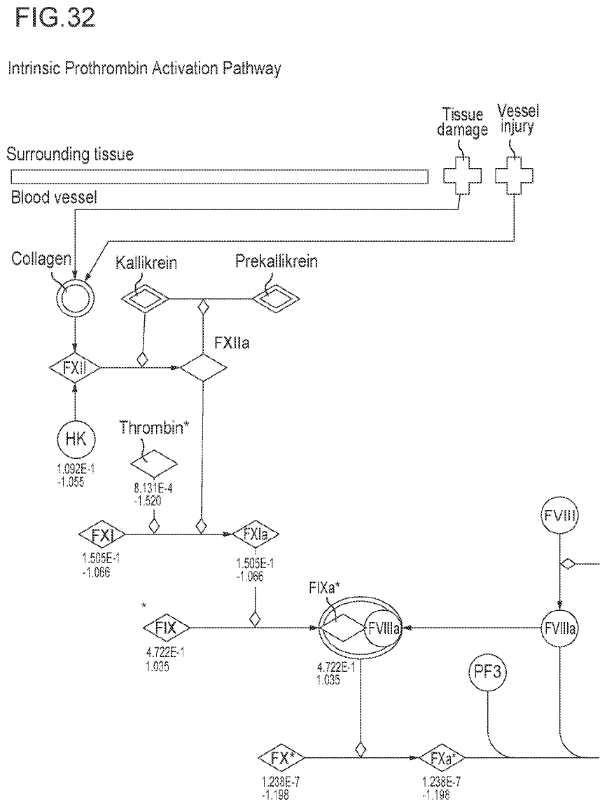

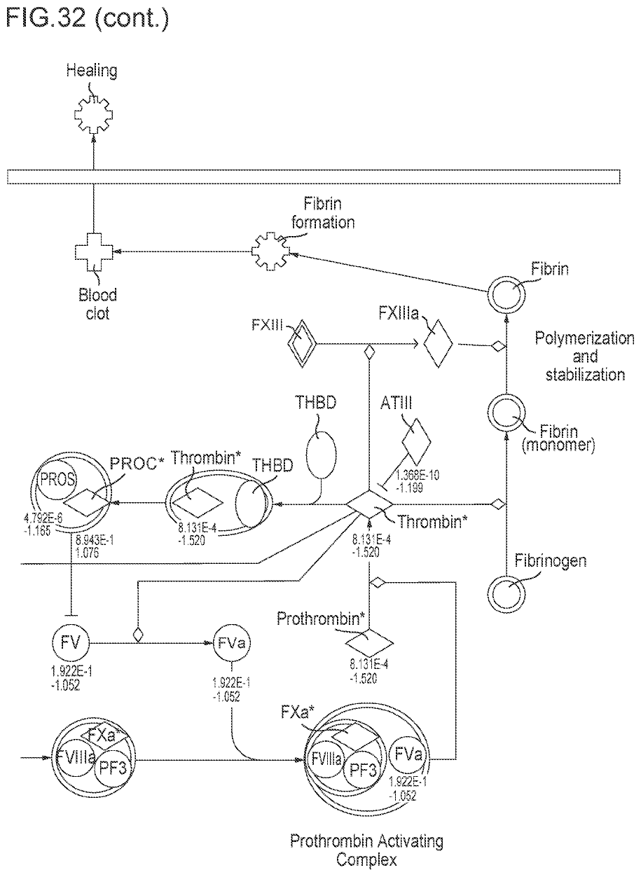

The intrinsic prothrombin activation pathway is illustrated in FIG. 32. Thrombin is a key coagulation enzyme. Among the many functions of thrombin are cleavage of fibrinogen to form fibrin and activation of platelets, both major constituents of normal and pathologic blood clots. The formation of fibrin clots proceeds through a tightly regulated series of reactions involving a group of plasma proteases and cofactors. Clot formation is essential for minimizing blood loss from an injured blood vessel (hemostasis), but pathologic fibrin formation and platelet activation may occlude vessels (thrombosis).

Plasma coagulation proceeds through a cascade of proteolytic reactions involving trypsin-like enzymes that form a biochemical amplifier, culminating in generation of sufficient thrombin to form a fibrin clot, There is an extrinsic pathway where initiation of fibrin formation occurs when plasma factor Vila forms a complex with the integral membrane protein tissue factor (TF).

In the intrinsic pathway, coagulation is initiated when factor XII is activated on a charged surface by a process called contact activation. Activation of factor XII is followed sequentially by activation of factor XI and factor IX. The intrinsic and extrinsic pathways converge at the level of factor X activation. Factor Xa activates prothrombin to thrombin in the presence of the cofactor factor Va, and thrombin subsequently converts fibrinogen to fibrin.

In humans, correlations between plasma levels of factors VIII, IX, and XI, and risk of venous thromboembolism have been demonstrated in large case controlled population studies. An association between factor XI and arterial disease has also been seen in several recent studies, which raise the possibility that factor XI may be more important for thrombus formation in the carotid artery or heart (the origins of most emboli that occlude cerebral vessels) than for thrombus formation at a site of plaque rupture in a coronary artery.

Protease-deficient mouse models have been used to elucidate the roles of the different factors in thromboembolic disease, Mice lacking factor IX, XI or XII have antithrombotic phenotypes. Inhibiting a protease in the intrinsic pathway may offer a strategy for preventing or treating arterial thrombosis.

### Genome

Genomics is a discipline in genetics that applies recombinant DNA, DNA sequencing methods, and bioinformatics to sequence, assemble, and analyze the function and structure of genomes (the complete set of DNA within a single cell of an organism). The field includes efforts to determine the entire DNA sequence of organisms and fine-scale genetic mapping. The field also includes studies of intragenomic phenomena such as heterosis, epistasis, pleiotropy and other interactions between loci and alleles within the genome.

Nucleic acid sequencing (e.g., genomic DNA, cDNA, rRNA, mRNA) in one embodiment is used to identify biomarkers in individuals with Down syndrome. Sequencing platforms include, but are not limited to, Sanger sequencing and high-throughput sequencing methods available from Roche/454 Life Sciences, Illumina/Solexa, Pacific Biosciences, Ion Torrent and Nanopore. The sequencing can be amplicon sequencing of particular DNA or RNA sequences or whole metagenome/transcriptome shotgun sequencing.

Traditional Sanger sequencing (Sanger et al. (1977) DNA sequencing with chain-terminating inhibitors. Proc Natl. Acad. Sci. USA, 74, pp. 5463-5467, incorporated by reference herein in its entirety) relies on the selective incorporation of chain-terminating dideoxynucleotides by DNA polymerase during in vitro DNA replication and is amenable for use with the methods described herein.

In another embodiment, the sample, or a portion thereof is subjected to extraction of nucleic acids, amplification of DNA of interest (such as the rRNA gene) with suitable primers and the construction of clone libraries using sequencing vectors. Selected clones are then sequenced by Sanger sequencing and the nucleotide sequence of the DNA of interest is retrieved.

454 pyrosequencing from Roche/454 Life Sciences yields long reads and can be used to identify biomarkers in individuals with Down syndrome (Margulies et al. (2005) Nature, 437, pp. 376-380; U.S. Pat. Nos. 6,274,320; 6,258,568; 6,210,891, each of which is herein incorporated in its entirety for all purposes). Nucleic acid to be sequenced (e.g., amplicons or nebulized genomic/metagenomic DNA) have specific adapters affixed on either end by PCR or by ligation. The DNA with adapters is fixed to tiny beads (ideally, one bead will have one DNA fragment) that are suspended in a water-in-oil emulsion. An emulsion PCR step is then performed to make multiple copies of each DNA fragment, resulting in a set of beads in which each bead contains many cloned copies of the same DNA fragment. Each bead is then placed into a well of a fiber-optic chip that also contains enzymes necessary for the sequencing-by-synthesis reactions. The addition of bases (such as A, C, G, or T) trigger pyrophosphate release, which produces flashes of light that are recorded to infer the sequence of the DNA fragments in each well. About 1 million reads per run with reads up to 1,000 bases in length can be achieved. Paired-end sequencing can be done, which produces pairs of reads, each of which begins at one end of a given DNA fragment. A molecular barcode can be created and placed between the adapter sequence and the sequence of interest in multiplex reactions, allowing each sequence to be assigned to a sample bioinformatically.

Illumina/Solexa sequencing produces average read lengths of about 25 basepairs (bp) to about 300 bp (Bennett et al. (2005) Pharmacogenomics, 6:373-382; Lange et al. (2014). BMC Genomics 15, p. 63; Fadrosh et al. (2014) Microbiome 2, p. Caporaso et al. (2012) ISME J, 6, p. 1621-1624; Bentley et al. (2008) Accurate whole human genome sequencing using reversible terminator chemistry. Nature, 456:53-59) and can be used to identify biomarkers in individuals with Down syndrome. This sequencing technology is also sequencing-by-synthesis but employs reversible dye terminators and a flow cell with a field of oligos attached. DNA fragments to be sequenced have specific adapters on either end and are washed over a flow cell filled with specific oligonucleotides that hybridize to the ends of the fragments. Each fragment is then replicated to make a cluster of identical fragments. Reversible dye-terminator nucleotides are then washed over the flow cell and given time to attach. The excess nucleotides are washed away, the flow cell is imaged, and the reversible terminators can be removed so that the process can repeat and nucleotides can continue to be added in subsequent cycles. Paired-end reads that are 300 bases in length each can be achieved. An Illumina platform can produce 4 billion fragments in a paired-end fashion with 125 bases for each read in a single run. Barcodes can also be used for sample multiplexing, but indexing primers are used.

The SOLiD (Sequencing by Oligonucleotide Ligation and Detection, Life Technologies) process is a “sequencing-by-ligation” approach, and can be used to identify biomarkers in individuals with Down syndrome (Peckham et al. SOLiD™ Sequencing and 2-Base Encoding. San Diego, Calif.: American Society of Human Genetics, 2007; Mitra et al. (2013) Analysis of the intestinal microbiota using SOLiD 16S rRNA gene sequencing and SOLiD shotgun sequencing. BMC Genomics, 14(Suppl 5): S16; Mardis (2008) Next-generation DNA sequencing methods. Annu Rev Genomics Hum Genet, 9:387-402; each incorporated by reference herein in its entirety). A library of DNA fragments is prepared from the sample to be sequenced, and are used to prepare clonal bead populations, where only one species of fragment will be present on the surface of each magnetic bead. The fragments attached to the magnetic beads will have a universal P1 adapter sequence so that the starting sequence of every fragment is both known and identical. Primers hybridize to the P1 adapter sequence within the library template. A set of four fluorescently labelled di-base probes compete for ligation to the sequencing primer. Specificity of the di-base probe is achieved by interrogating every 1st and 2nd base in each ligation reaction. Multiple cycles of ligation, detection and cleavage are performed with the number of cycles determining the eventual read length. The SOLID platform can produce up to 3 billion reads per run with reads that are 75 bases long. Paired-end sequencing is available and can be used herein, but with the second read in the pair being only 35 bases long. Multiplexing of samples is possible through a system akin to the one used by Illumina, with a separate indexing run.

The Ion Torrent system, like 454 sequencing, is amenable for use to identify biomarkers in individuals with Down syndrome. It uses a plate of microwells containing beads to which DNA fragments are attached. It differs from all of the other systems, however, in the manner in which base incorporation is detected. When a base is added to a growing DNA strand, a proton is released, which slightly alters the surrounding pH. Microdetectors sensitive to pH are associated with the wells on the plate, and they record when these changes occur. The different bases (A, C, G, T) are washed sequentially through the wells, allowing the sequence from each well to be inferred. The Ion Proton platform can produce up to 50 million reads per run that have read lengths of 200 bases. The Personal Genome Machine platform has longer reads at 400 bases. Bidirectional sequencing is available. Multiplexing is possible through the standard in-line molecular barcode sequencing.

Pacific Biosciences (PacBio) SMRT sequencing uses a single-molecule, real-time sequencing approach and in one embodiment, is used to identify biomarkers in individuals with Down syndrome. The PacBio sequencing system involves no amplification step, setting it apart from the other major next-generation sequencing systems. In one embodiment, the sequencing is performed on a chip containing many zero-mode waveguide (ZMW) detectors. DNA polymerases are attached to the ZMW detectors and phospholinked dye-labeled nucleotide incorporation is imaged in real time as DNA strands are synthesized. The PacBio system yields very long read lengths (averaging around 4,600 bases) and a very high number of reads per run (about 47,000). The typical “paired-end” approach is not used with PacBio, since reads are typically long enough that fragments, through CCS, can be covered multiple times without having to sequence from each end independently. Multiplexing with PacBio does not involve an independent read, but rather follows the standard “in-line” barcoding model.

In one embodiment, a biomarker can comprise the ITS genomic region. The ITS region has significant heterogeneity in both length and nucleotide sequence. The use of a fluorescence-labeled forward primer and an automatic DNA sequencer permits high resolution of separation and high throughput. The inclusion of an internal standard in each sample provides accuracy in sizing general fragments.

In another embodiment, fragment length polymorphism (RFLP) of PCR-amplified rDNA fragments, otherwise known as amplified ribosomal DNA restriction analysis (ARDRA), is used as biomarkers (Massol-Deya et al. (1995). Mol. Microb. Ecol. Manual. 3.3.2, pp. 1-18, incorporated by reference in its entirety for all purposes). rDNA fragments are generated by PCR using general primers, digested with restriction enzymes, electrophoresed in agarose or acrylamide gels, and stained with ethidium bromide or silver nitrate.

### Functional Genome

Functional genomics is a field of molecular biology that uses the vast wealth of data produced by genomic projects (such as genome sequencing projects) to describe gene (and protein) functions and interactions, Contrary to classical genomics, functional genomics focuses on the dynamic aspects such as gene transcription, translation, and protein-protein interactions, as opposed to the static aspects of the genomic information such as DNA sequence or structures. Functional genomics attempts to answer questions about the function of DNA at the levels of genes, RNA transcripts, and protein products. A key characteristic of functional genomics studies is a genome-wide approach to these questions, generally involving high-throughput methods rather than a more traditional “gene-by-gene” approach. Given the vast inventory of genes and genetic information it is advantageous to use genetic screens to provide information of what these genes do, what cellular pathways they are involved in and how any alteration in gene expression can result in a particular biological process.

Functional genomic screens and libraries attempt to characterize gene function in the context of living cells and hence are likely to generate biologically significant data. There are three key elements for a functional genomics screen: a good reagent to perturb the gene, a good tissue culture model and a good readout of cell state. Gene perturbation allows the understanding of that gene's function. Precise genome targeting technologies enable systematic reverse engineering of causal genetic variations by allowing selective perturbation of individual genetic elements, as well as to advance synthetic biology, biotechnological, and medical applications. Genome-editing techniques include designer zinc fingers, transcription activator-like effectors (TALEs), homing meganucleases and CRISPR-Cas.

### Proteome

The term “proteomics” was coined to make an analogy with genomics, and while it is often viewed as a continuation of genomics, proteomics is much more complicated than genomics. Most importantly, whilst the genome is a rather constant entity, the proteome differs from cell to cell and is constantly changing through its biochemical interactions with the genome and the environment. One organism will have radically different protein expression in different parts of its body, in different stages of its life cycle and in different environmental conditions.

The protein map of a biological system, including a cell, sub-cellular fraction or expression media, can be referred to as a proteome. Proteomics, or analysis of the proteome of a biological system, examines protein expression profiling and cellular or tissue protein identification from samples that are obtained under various specified conditions. Proteomics has an enormous breadth of application ranging from investigation and identification of biomarkers, molecules that are indicative of a particular pathological state, which in turn can be used for diagnostic purposes and targets for therapeutic intervention, Proteome analysis allows the investigator to obtain information on protein identity, protein-protein interaction, the level of protein expression and protein expression profiling, protein trafficking and turnover, protein variants, and protein post-translational modifications.

Traditionally, proteomics combines two-dimensional electrophoresis (2-DE), a high-resolution protein separation technique, with mass spectrometry (MS). Proteomics research is targeted towards characterization of the proteins encoded by a particular genome and its changes under the influence of biological stimulation. Proteomics also involves the study of non-genome encoded events such as the post-translation modification of proteins, interactions between proteins, and the location of proteins within the cell. The study of gene expression at the protein level is important because many of the most important cellular activities are directly regulated by proteins in the cell rather than by gene activity. Also, the protein content of a cell is highly relevant to drug discovery and drug development efforts since most drugs are designed to target proteins. Therefore, the information gained from proteomics is expected to greatly boost the number of drug targets,

Although two-dimensional gel electrophoresis is one of the most powerful methods in the current study of proteomics, this method is labor-intensive, time consuming, and limited in sensitivity. The two-dimensional gel electrophoresis method also suffers from poor reproducibility. To avoid the aforementioned disadvantages of two-dimensional gel electrophoresis, microchip-based separation devices (microarrays) have been developed for rapid analysis of large numbers of samples. Compared to conventional separation columns or devices, microarrays have higher sample throughput, reduced sample and reagent consumption, and reduced chemical waste. Such devices are capable of fast analyses and provide improved precision and reliability compared to the conventional analytical instruments. The cDNA microarray methodologies provide parallel and quantitative expression profiles of thousands of genes, which when combined with bioinformatics tools, can identify genes in a biologic pathway, characterize the function of novel genes, and detect disease subclasses.

Mass spectrometry is a technique that measures m/z (mass-to-charge) intensity pairs of an ionizable substance, The m/z-intensity pair or pairs of an analyte provides a signature distinguishing the analyte from other substances having a different m/z-intensity pair' or pairs. The intensity of an analyte's m/z-intensity pair changes with the analyte's abundance within the response range of the instrument, Techniques and equipment for generating mass spectrometry data are well known in the art. Examples of ionization techniques that can be employed include electronspray ionization, matrix-assisted laser desorption/ionization (MALDI), surface enhanced laser desorption/ionization (SELDI), electron impact ionization, chemical ionization, and photoionization,

Recently, a chip-based proteomics approach has been introduced using biomolecular interaction analysis-mass spectrometry (BIA-MS) in rapidly detecting and characterizing proteins present in complex biological samples at very low levels. One of the most powerful techniques is Surface Enhanced Laser Desorption/lonization Time-of-Flight Mass Spectrometry (SELDI-TOF-MS) technology, which has been commercially embodied in Ciphergen's ProteinChip® Biomarker System. The system uses chemically (cationic, anionic, hydrophobic, metal, etc.) or biochemically (antibody, DNA, enzyme, receptor, etc.) treated surfaces for specific interaction with proteins of interest, followed by selected washes for SELDI-TOF-MS detection. Surface-Enhanced Laser Desorption/Ionization (SELDI) was invented in the late 1980's. When coupled to a time-of-flight mass spectrometer (TOF), SELDI provides a means to rapidly analyze molecules retained on a chip. The power of the system incorporates straightforward sample preparation with on-chip capture (binding) and detection for protein discovery, protein purification, and protein identification from small samples, allowing rapid analysis and assay development on a single platform,

Proteins present in complex biological samples can also be captured using an aptamer-based technology. SELEX (Systematic Evolution of Ligands by EXponential enrichment) allows the selection of nucleic acid ligands, or aptamers, with high binding affinity for target molecules, Aside from their high affinities, aptamers possess exquisite specificities for their targets as several crystal structures of aptamer-target complexes have shown precisely folded entities that recognize their targets through shape complementarity. The stability of aptamers has been improved by the use of modified nucleoside triphosphates. Thus SELEX now entails use of 2′-aminopyrimidine and 2′-fluoropyrimidine RNA libraries. One of the major advantages of SELEX compared with other combinatorial methods is the enormous size of the initial random libraries that can be generated and screened (typically 10¹⁵ molecules), which is about 100,000 times larger than peptide-based libraries. However, the chemical diversity of nucleic acid libraries is substantially lower compared with protein-based libraries. A new class of ligands have been produced with the judicious introduction of functional groups absent in natural nucleic acids (Rohloff et al, (2014), Molecular Therapy—Nucleic Acids (2014) 3, e201). Hydrophobic aromatic side chains at the 5-position of uracil have the most profound influence on the success rate of SELEX and allow the identification of ligands with very low dissociation rate constants (named Slow Off-rate Modified Aptamers or SOMAmers). Such modified nucleotides create unique intramolecular motifs and make direct contacts with proteins. Importantly, SOMAmers engage their protein targets with surfaces that have significantly more hydrophobic character compared with conventional aptamers, thereby increasing the range of epitopes that are available for binding. The current collection of SOMAmers recognize over 3,000 human proteins encompassing major families such as growth factors, cytokines, enzymes, hormones, and receptors.

### Metabolome

The invention pertains, at least in part, to the generation and the analysis of small molecule profiles of cells, cellular compartments, and specific organelles (e.g., mitochondria, Golgi, endoplasmic reticulum, cytoplasm, nucleus, etc.). Small molecule profiles allow for the identification and interrogation of inventories of small molecules (e.g., the metabolome) to find, for example, disease-relevant small molecules as well as potential targets for drug design.

Small molecule profiles of cells and organelles can be used directly to identify drug candidates. Small molecule profiling can either eliminate entirely or accelerate the process of identifying genes and proteins associated with a condition and/or disease. In one embodiment of the invention, the methods of the invention include, for example, comparing small molecule profiles of diseased cells, cellular compartments, and organelles to standard profiles of healthy cells, cellular compartments, and organelles. Therefore, if a particular diseased cell, cellular compartment, or organelle was found to be deficient in a particular compound, the deficiency may be overcome by simply administering the compound or an analogue thereof. Metabolomics offers a new route to the identification of potentially therapeutic agents and targets.

Small molecule profiling allows one to investigate the very biochemical pathway (e.g., cellular metabolites) involved in the condition and/or disease by comparing small molecule profiles of cells, cellular compartments, or organelles with those of cells, cellular compartments, or organelles treated with toxins, chemical agents or other therapeutic agent (or derived from an organism treated with the agent or drug).

The invention also includes methods for identifying potential cell drug targets (e.g., cellular components which interact with the labeled small molecules). This method is particularly useful because it can identify components which are known to interact with disease relevant small molecules. Therefore, targets identified through this method are “pre-validated,” and some of the guess work surrounding the choice of target is eliminated. In a further embodiment, this method can be used in conjunction with conventional genomics as a further validation step to identify targets for further research.

Unlike genomics, small molecule profiling is not limited to disease states with a genetic component. Many disease states are not genetically determined and genomics offers little to those suffering or at risk of suffering from non-genetic linked disease states. Therefore, there is a need for a comprehensive method to study the effects of nongenetic factors on cells and living systems.

Small molecule profiling of cells, organelles, or extracellular material can be used to study both genetic and non-genetically linked disease states. For example, methods of the invention can be used to identify small molecules associated with, for example, Alzheimer's, diabetes, autism, congenital heart defects, celiac disease, thyroid dysfunction, autoimmune disorders, vision problems, hearing problems, intestinal atresia and sleep apnea.

In addition, metabolomics can be used in tandem with genomics and/or proteomics. For example, small molecule profiles can be used to identify small molecules regulated, modulated, or associated with genetic modification or alterations of cells, both engineered and naturally occurring.

In addition, metabolomics can also be applied to the field of predictive medicine. For example, the invention pertains to diagnostic assays, prognostic assays, pharmacometabolomics, and the monitoring of clinical trials which are used for prognostic (predictive) purposes to treat an individual prophylactically, based on an individual's small molecule profile. Unlike pharmacogenetics, which is limited to genetic factors, pharmacometabolomics is able to predict an individual's response to a drug based not only on genetic factors, but also non-genetic factors, such as other drugs in the patient's body, the patient's current state of health, etc. Pharmacometabolomics allows for the use of a subject's small molecule profile to deliver the right drug to the right patient. Subjects respond differently to drugs based on their small molecule profiles.

The invention pertains, at least in part, to the generation of small molecule profiles of samples, cells, and cellular compartments. Small molecule profiles “fingerprint” the cell or cellular compartment and identify the presence, absence or relative quantity of small molecules. The small molecule profiles of the cells or cellular compartments may be obtained through, for example, a single technique or a combination of techniques for separating and/or identifying small molecules known in the art. Examples of separation and analytical techniques which can be used to separate and identify the compounds of the small molecule profiles include: HPLC, TLC, electrochemical analysis, mass spectroscopy, refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), Light Scattering analysis (LS) and other methods known in the art. Preferably, the methods of the invention detect both electrically neutral as well as electrochemically active compounds. Detection and analytical techniques can be arranged in parallel to optimize the number of molecules identified.

The term “cells” includes eukaryotic cells and human cells. The term cells includes transgenic cells from cultures. The cells may be from a specific tissue, body fluid, organ (e.g., brain tissue, nervous tissue, muscle tissue, retina tissue, kidney tissue, liver tissue, etc.), or any derivative fraction thereof. The term includes healthy cells, transgenic cells, cells affected by internal or exterior stimuli, cells suffering from a disease state or a disorder, cells undergoing transition (e.g., mitosis, meiosis, apoptosis, etc.), etc.

In a further embodiment, the samples are obtained from a specific cellular compartment. The term “cellular compartment” includes organelles (such as mitochondria, Golgi apparatus, centrioles, chloroplasts), the nucleus, the cytoplasm (optionally including the organelles), and other cellular regions capable of being isolated. In one embodiment, the cellular compartment is the entire cell.

The analysis of a particular cellular compartment has many advantages over analysis of whole cells, whole cell lysates, body fluids, etc. For example, often the mechanism of action of a drug, a toxic compound, etc. is directed to a specific cellular function, such as, for example, the electron transport chain in the mitochondria, nucleic acid replication in the nucleus, etc. By isolating the specific cellular compartment or organelle (e.g., mitochondria, nuclei, Golgi apparatus, endoplasmic reticulum, ribosomes, etc.), it is possible to narrow the focus of the profile to small molecules involved in the relevant pathway. Previously, metabolome studies have been complicated by the large number of chemical species present in a given sample. By narrowing the scope of the study to the particular organelle, researchers will be able to study the pathway of interest in more detail without irrelevant molecules present in interstitial fluid, blood, spinal fluid, saliva, etc.

The term “small molecules” includes organic and inorganic molecules which are present in the cell, cellular compartment, or organelle. The term does not include large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000). The small molecules of the cell are generally found free in solution in the cytoplasm or in other organelles, such as the mitochondria, where they form a pool of intermediates which can be metabolized further or used to generate large molecules, called macromolecules. The term “small molecules” includes signaling molecules and intermediates in the chemical reactions that transform energy derived from food into usable forms. Examples of small molecules include sugars, fatty acids, amino acids, nucleotides, intermediates formed during cellular processes, and other small molecules found within the cell. In one embodiment, the small molecules cf the invention are isolated.

The term “metabolome” includes all of the small molecules present in a given organism. The metabolome includes both metabolites as well as products of catabolism, In one embodiment, the invention pertains to a small molecule profile of the entire metabolome of a species. In another embodiment, the invention pertains to a computer database (as described below) of the entire metabolome of a species, e.g., an animal, e.g., a mammal, e.g., a mouse, rat, rabbit, pig, cow, horse, dog, cat, bear, monkey, and, preferably, a human. In another embodiment, the invention pertains to a small molecule library of the entire metabolome of an organism (as described below), e.g., a mammal, e.g., a mouse, rat, rabbit, pig, cow, horse, dog, cat, bear, monkey, and, preferably, a human.

The language “small molecule profile” includes the inventory of small molecules in tangible form within a targeted cell, tissue, organ, organism, or any derivative fraction thereof, e.g., cellular compartment, that is necessary and/or sufficient to provide information to a user for its intended use within the methods described herein, The inventory would include the quantity and/or type of small molecules present. The ordinarily skilled artisan would know that the information which is necessary and/or sufficient will vary depending on the intended use of the “small molecule profile.” For example, the “small molecule profile,” can be determined using a single technique for an intended use but may require the use of several different techniques for another intended use depending on such factors as the disease state involved, the types of small molecules present in a particular targeted cellular compartment, the cellular compartment being assayed per se., etc.

The relevant information in a “small molecule profile” also may vary depending on the intended use of the compiled information, e.g. spectra. For example for some intended uses, the amounts of a particular small molecule or a particular class of small molecules may be relevant, but for other uses the distribution of types of small molecules may be relevant.

The ordinarily skilled artisan would be able to determine the appropriate “small molecule profiles” for each method described herein by comparing small molecule profiles from Down syndrome subjects with typical and/or healthy subjects. These comparisons can be made by individuals, e.g., visually, or can be made using software designed to make such comparisons, e,g., a software program may provide a secondary output which provides useful information to a user. For example, a software program can be used to confirm a profile or can be used to provide a readout when a comparison between profiles is not possible with a “naked eye”. The selection of an appropriate software program, e.g., a pattern recognition software program, is within the ordinary skill of the art. An example of such a program is Pirouette. It should be noted that the comparison of the profiles can be done both quantitatively and qualitatively.

To create a small molecule profile, organs, cells, cellular compartments, or organelles are homogenized in standard ways know for those skilled in the art. Different fractionation procedures can be used to enrich the fractions for small molecules. The small molecules obtained will then be passed over several fractionation columns. The fractionation columns will employ a variety of detectors used in tandem or parallel to generate the small molecule profile for the organ, cell, cellular compartment, or organelle.

### Methods of Identification of Disease-Relevant Small Molecules

In another embodiment, the invention includes a method of identifying disease-relevant small molecules. The method includes comparing small molecule profiles of diseased cells, cellular compartments, or organelles to a standard profile of a healthy cell, cellular compartment, or organelle. The method also involves identifying the small molecules which are present in aberrant amounts in the diseased small molecule profile. The small molecules present in aberrant amounts in the diseased cells are “disease-relevant small molecules.”

The language “disease-relevant small molecules” includes both small molecules present in aberrant amount in diseased small molecule profiles and, in addition, small molecules which are potentially involved in disease initiation, progression or prediction. The term also may include small molecules which when modulated, result in the lessening or curing of at least one symptom of a disease, The disease relevant small molecules are ideal drug candidates in the screening assays discussed elsewhere in the application,

For example, identified disease relevant small molecules may be screened using in vitro or in vivo assays known in the art to determine biological activity, The biological activity of disease relevant small molecules can also be pinpointed by using screening assays against protein targets which have been implicated in the disease state. In another embodiment, the biological activity of disease relevant small molecules can be determined using cell-based assays, e,g., tumor cell assays (Lillie et al. Cancer Res. 53(13):3172-8 (1993)). The disease relevant small molecules can also be tested for neuronal protection activity by exposing primary or cultured neurons to the compounds and toxic agents, such as glutamate, and identifying the compounds which protect the neurons from death. Animal models can also be used to further identify the biological activity of disease relevant small molecules. For example, animal models of Huntington's disease, Parkinson's disease, and ALS can be used to identify small molecules useful as neuroprotective agents. (Kilvenyi, Nature Med. 5:347-350 (1999); Mathews et al, Experimental Neurology 157:142-149 (1999)). In a further embodiment, the disease relevant small molecules can be chemically modified to further enhance their pharmaceutical or nutraceutical properties.

The language “aberrant levels” includes any level, amount, or concentration of a small molecule in a cell, cellular compartment, or organelle which is different from the level of the small molecule of a standard sample.

The term “standard profile” includes profiles derived from healthy cells, advantageously from a similar origin as the source. In one embodiment, the standard profile is an average of many samples of a certain cell type and/or a certain cellular compartment. In another embodiment, the standard profile may be derived from a typical individual. Or, in another embodiment the standard profile can be an average of the profiles obtained from numerous sources, e.g., the standard profile may be an average of small molecule profiles obtained from 2 or more typical individuals. The standard profile can be a small molecule profile of a certain cellular compartment or from a certain subset of cells. In one embodiment, the invention pertains to the standard profile of healthy cells. Advantageously, the small molecules with aberrant levels in the sample are identified, e.g., HPLC, TLC, electrochemical analysis, mass spectroscopy, refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), Light Scattering analysis (LS) and other methods known in the art. In one embodiment, the small molecule profile of the sample, cell, or cellular compartment, is compared to the standard profile by using subtracting one profile from the other, The compounds which are present in aberrant amounts can then be used in drug design to identify deregulated cellular components. Standard profiles can also be made of the effects of certain agents (e.g., drugs, therapeutic agents, toxins, etc.) on both healthy and diseased cells (e.g., cells diseased with the type of disease treated by the therapeutic agent).

Furthermore the language “standard profile” includes information regarding the small molecules of the profile that is necessary and/or sufficient to provide information to a user for its intended use within the methods described herein. The standard profile would include the quantity and/or type of small molecules present. The ordinarily skilled artisan would know that the information which is necessary and/or sufficient will vary depending on the intended use of the “standard profile.” For example, the “standard profile,” can be determined using a single technique for an intended use but may require the use of several different techniques for another intended use depending on such factors as the types of small molecules present in a particular targeted cellular compartment, the cellular compartment being assayed per se., etc.

The relevant information in a “standard profile” also may vary depending on the intended use of the compiled information, e.g. spectra, For example for some intended uses, the amounts of a particular small molecule or a particular class of small molecules of the standard profile may be relevant, but for other uses the distribution of types of small molecules small molecules of the standard profile may be relevant,

Furthermore, comparison of the standard profiles to profiles from diseased cells can be used to identify small molecules deregulated in the disease state. The small molecules identified can be used to guide the drug discovery effort. For example, the small molecules present in aberrant levels in the sample cells, can be identified and used as pharmaceutical or nutraceutical agents. For example, if a patient is suffering from a disease state associated with an aberrantly low level of a certain compound, the compound or a precursor thereof may be tested in an assay that mimics the disease state. In another embodiment, the small molecules present in aberrant amounts may be used as targets for drug design to develop agents with enhanced activity, e.g., enhanced activity to treat the disease state associated with the aberrant levels of the small molecule, Additionally libraries of small molecules based on the structures of the small molecules present in aberrant amounts can be used to develop more potent therapeutics. The cellular targets and pathways could also be used to guide drug design,

In a further embodiment, the invention pertains to a method for treating a patient with a deficiency in certain disease relevant small molecules. The method includes obtaining cells from the patient, obtaining the small molecule profile of either a particular organelle (e.g., mitochondria, nucleus, cytoplasm, Golgi apparatus, endoplasmic reticulum, etc.) or a cell, comparing the small molecule profile with a standard profile, determining a deficiency in the patient's small molecule profile of a certain disease relevant small molecule, and administering the disease relevant small molecule to the patient.

In a further embodiment, the invention features diagnostic assays for the detection of disease states. For example, the method includes identifying a small molecule which is present in aberrant amounts in a particular disease state, e.g., by comparing standard profiles of diseased cells or cellular compartments with healthy cells or cellular compartments to identify compounds which are present in aberrant amounts in the diseased cell or cellular compartment. The method also involves designing a reagent that specifically reacts with the compound present in aberrant amounts to indicate the presence or absence of the compound, and therefore, the presence or the absence of the disease. The invention also pertains to kits which include the reagent and instructions for its use to diagnose the disease.

Methods of Identifying the Effect of Chemical Agents on Small Molecule Profiles of Cells, Cellular Compartments, Organelles, and Extracellular Material

In another aspect, the invention pertains to the comparison of small molecule profiles of cells, cellular compartments, organelles, or extracellular material with those of cells, cellular compartments, organelles, or extracellular material treated with toxins, chemical agents or therapeutic agent (or derived from an organism treated with the agent or drug). In one embodiment, the cells, cellular compartments, organelles, or extracellular material are diseased (or derived from a diseased organism) and are treated with a therapeutic agent which is known to modify or treat that disease. For example, the small molecule profile of a cell treated with a therapeutic agent, chemical agent, or toxin, can be compared to the small molecule profile of a normal cell, e.g., a healthy cell of similar lineage, or a diseased cell of similar lineage which was not treated with the therapeutic agent, chemical agent, or toxin. Examples of toxins include bacterial toxins such as endotoxins and exotoxins, such as cholera toxin, diptheria toxin, verotoxin, enterotoxin, etc. In a further embodiment, the cells are genetically altered.

Extracellular material include blood, sera, spinal fluid, brain fluid, saliva, urine, semen, mucosal excretions, etc. Small molecule profiles of these extracellular materials of a particular organism may be obtained in a similar fashion to small molecule profiles of cells, cellular compartments and organelles.

In addition, subtraction profiles can be obtained by subtracting the non-treated profile or a standard profile with the small molecule profile from a treated cell, cellular compartment, organelle, or extracellular fluid. The subtraction profiles can then be used to identify certain small molecules the presence or the absence of which may indicate the efficacy or the toxicity of the compound. The subtraction profiles can be made using, for example, computer programs known to those of skill in the art, e.g., pattern recognition software program. An example of such a program is Pirouette. It should be noted that the comparison of the profiles can be done both quantitatively and qualitatively.

In a further embodiment, the invention pertains to certain small molecules which indicate the efficacy or the toxicity of the compound. The invention also applies to assays which can be developed to indicate the presence or absence of these certain small molecules. For example, if the presence of a certain small molecule is essential for the efficacy of a particular therapeutic compound, then an assay can be developed to quickly determine the presence or absence of this certain small molecule in cell samples treated with test compounds. This can be both an effective and inexpensive method to determine the potential efficacy of compounds. It can be used alone or in combination with traditional drug screening assays such as, for example, binding assays and other enzymatic assays.

For example, in search of molecules with anti-tumor activity, small molecule profiles could be taken of cells at certain intervals after being treated with a known anti-tumor drug (e.g., taxol, cisplatin, adriamycin, etc.). Comparison of the small molecule profiles of these cells could lead to the identification of small molecules regulated by these drugs. The identified small molecules could then be used to guide drug discovery by pointing to pathways which could be targeted for drug design or by using them as therapeutic or nutraceutical agents.

The invention also includes a method for determining the toxicity of a test compound, e.g., a compound in development as a therapeutic agent. The method includes culturing cells, contacting a portion of the cells with the test compound, taking small molecule profiles of both the cells contacted with the test compound, taking the small molecule profiles of cells not contacted with the test compound, and comparing the profiles to either each other or profiles from cells contacted with a known therapeutic agent or cells contacted with a known toxin. The method also can include a step of purifying a particular organelle of interest from the cells and obtaining the small molecule profile of the particular organelle of interest (e.g., nuclei, mitochondria, Golgi apparatus, endoplasmic reticulum, ribosome, etc.). Extracellular material also may be monitored in a similar fashion.

In a further embodiment, the invention pertains to a method for reducing side effects of drugs under development. For example, cells can be cultured, contacted with the test compound, the small molecule profile can be generated, and compared to the profiles of known toxins and therapeutic agents. Changes then can be made to the structure of the test compound to reduce the side effects. For example, in order to test for liver toxicity, the compound may be incubated in a liver cell culture to mimic the biotransformation that occurs in the liver. The small molecule profiles of cells and organelles in the treated and untreated liver cultures can be compared to the small molecule profiles of known toxins. Both the total cellular small molecule profile could be compared or the small molecule profile of a particular organelle, e.g., mitochondria, Golgi apparatus, nuclei, ribosomes, endoplasmic reticulum, etc. could be used for comparison.

The methods of the invention are particularly useful because they offer a quick and relatively inexpensive method to determine whether a certain test compound is likely toxic to a body organ, such as the liver. This allows for pharmaceutical companies to quickly screen and identify compounds which are toxic and to direct their research towards non-toxic compounds.

The methods and small molecule profiles of the invention may also be used to rescue drugs, e.g., drugs which fail a particular step in the clinical or pre-clinical trial procedure. The failed drug can be exposed to cells or a test organism and small molecule profiles of the cells, cellular compartments, organelles, extracellular fluid, etc. can be taken and compared to those of known toxins, known therapeutic agents, etc. to pinpoint the reason for failure of the drug. Small molecule profiles of various organs can also be taken if it is advantageous for the study (e.g., small molecule profiles can be taken from muscle, brain, retinal, nerve, heart, lung, stomach, colon, skin, breast, fatty tissue, blood, etc.) Then the drug can be redesigned to avoid its previous adverse effects,

The methods and small molecule profiles of the invention can also be used to “reposition” drugs.

The term “reposition” refers to discovering new uses for an agent. In one embodiment, a dose of an agent is administered to a subject (e.g., a human or other animal, healthy or diseased) and small molecule profiles are then taken from various organs, tissues, cells, cellular compartments, organelles, and/or extracellular fluid of the subject to determine what tissues, organs, cells, cellular compartments, organelles, and/or extracellular fluids are being affected by the administration of the agent.

Assays for Identifying Potential Cell Drug Targets Using Labeled Disease Relevant Small Molecules

In another embodiment, the invention also pertains to methods for identifying potential cell drug targets (e.g., cellular components which interact with the labeled small molecules). This method is particularly useful because it can identify components which are known to interact with disease relevant small molecules. Therefore, targets identified through this method are “pre-validated,” and some of the guess work surrounding the choice of target is eliminated. In a further embodiment, this method can be used in conjunction with conventional genomics as a further validation step to identify targets for further research.

The method includes obtaining a cell from a source, obtaining samples of small molecules from the cell; testing the samples for biological activity; identifying the biologically active small molecules of the samples; labeling the biologically active small molecules; contacting the labeled small molecules with cellular components; and identifying interactions between cellular components and said labeled small molecules. The invention includes the identified cell drug targets as well as the identified biologically active small molecules.

In another embodiment, the invention includes a method for identifying potential cell drug targets. The method includes contacting a labeled disease relevant small molecule with cellular components; and identifying interactions between said cell components and the labeled disease-relevant small molecule.

The labeled small molecules also include labeled “disease-relevant small molecules,” identified by any of the techniques described herein (e.g., comparison of small molecule profiles in healthy and diseased cells, etc.). In another embodiment, the method includes contacting a labeled disease relevant small molecule with cellular components, and identifying the interactions between the cellular components and the labeled disease relevant small molecule.

The term “label” includes any moieties or molecules which enhance the ability of the labeled small molecules to be detected, Examples of suitable labels are well known in the art, such as radiolabels and fluorescent labels. The term “label” includes direct labeling of the small molecule by radiolabeling, coupling (i.e., physically linking) a detectable substance (e.g., a fluorescent moiety) to the small molecule, and indirect labeling of the small molecule by reacting the small molecule with another reagent that is directly labeled. Examples of indirect labeling include detection of a small molecules by labeling it with biotin such that it can be detected with fluorescently labeled streptavidin. In one embodiment, the small molecules are fluorescently labeled or radiolabeled.

The term “cellular components” includes material derived from cells. The cellular components can be purified or crude cellular extracts. The cellular components can be derived from one type of cell, or even a specific cellular compartment such as an organelle (e.g., mitochondria, nucleus, cytoplasm). Furthermore, the term includes both natural proteins found within biological systems and chimeric and other engineered proteins. In one embodiment, the term “cellular component” includes cellular receptors. The term also includes natural and unnatural polysaccharides and nucleic acids. In one embodiment, the term “cellular component” is a crude cellular extract from a human cell, The term “cellular component” includes “targets.”

Samples of the invention that bind to cellular components can be identified by preparing a reaction mixture of the cellular components and the samples under conditions and for a time sufficient to allow the components and the sample to interact and bind, thus forming a complex which can be removed and/or detected in the reaction mixture. The cellular components used can vary depending upon the goal of the screening assay. In one embodiment, the sample of the invention is an isolated, labeled small molecule, e.g., a disease relevant small molecule, a small molecule with biological activity or another small molecule which is present in aberrant levels in disease states. The assay can be used to determine which cellular components the small molecule interacts with. The identified cellular components which interact with the small molecule can then be used for drug design,

In a further embodiment, the cellular components are a nucleic acid array. High density arrays of nucleic acids (such as cDNA's and synthetic oligonucleotides) allow for a high degree of automation, repetitive analysis and duplication at minimal cost (Fraser, Electrophoresis, 18:1207-1215 (1997)). The development of recent technology has provided methods for making very large arrays of oligonucleotide probes in very small areas (see, for example, U.S. Pat. No. 5,143,854, WO 90/15070 and WO 92/10092, each of which is incorporated herein by reference). In one embodiment, the nucleic acids of the array are human genes. Examples of nucleic acid arrays include those mentioned in U.S. Pat. No. 6,027,880 and U.S. Pat. No. 5,861,242. The nucleic acids also can be representative of RNA molecules present in a cell, tissue or organ (e.g., the “transcriptome”, see Hoheisel, J. et al. Trends Biotechnol. 15:465-469 (1997); Velculescu, Cell, 88:243-251 (1997)). In one embodiment, the nucleic acids are in array.

In another further embodiment, the cellular components are a protein array. Examples of protein arrays include those employing conventional protein separation techniques, such as 2-dimensional gel electrophoresis, chromatographic procedures (e.g., FPLC, SMART by Pharmacia, Uppsala, Sweden), capillary electrophoretic techniques and mass spectrometry. In another embodiment, the protein array is a soup of proteins that contains a significant portion of the diversity encoded by a genome (see WO 99/39210).

In a further embodiment, the cellular components are a 2D protein gel. The 2D protein gel may be a complete or an incomplete set of the protein molecules present in a cell, tissue or organ (e.g., the proteome, see Sagliocco, et al. Yeast 12, 1519-1534 (1996); Shevalanko, et al. Porch. Nat. Acad. Sci. 93, 14440-14445 (1996)). Labeled biologically active small molecules previously identified through methods of the invention can then be contacted with the 2D gels and interactions between the labeled small molecules and the protein of the 2D gel can be detected.

The proteins identified through this method can then be further tested for biological activity, e.g., biological activity relating to that of the small molecule, e.g., through knock-out mice, inhibition studies, and other techniques known in the art. Furthermore, the identified proteins can then be used in drug design to identify other molecules (either naturally occurring or chemically synthesized) which bind or interact with the protein which may have advantageous characteristics (e.g., enhanced biological activity, less toxic side effects).

Predictive Medicine and Pharmacometabolomics

The present invention also pertains to the field of predictive medicine in which diagnostic assays, prognostic assays, pharmacometabolomics, and monitoring clinical trials are used for prognostic (predictive) purposes to thereby treat an individual prophylactically. Accordingly, one aspect of the present invention relates to diagnostic assays for determining small molecule profiles, in the context of a biological sample (e.g., blood, serum, cells, tissue, cellular organelles) to thereby determine whether an individual is afflicted with a disease or disorder, or is at risk of developing a disorder, associated with aberrant levels of small molecules. The invention also provides for prognostic (or predictive) assays for determining whether an individual is at risk of developing a disorder associated with relevant small molecules. For example, aberrant levels of small molecules can be profiled from a biological sample. Such assays can be used for prognostic or predictive purpose to thereby prophylactically treat an individual prior to the onset of a disorder characterized by or associated with a relevant small molecule.

Another aspect of the invention provides methods for determining small molecule profiles of an individual to thereby select appropriate therapeutic or prophylactic agents for that individual (referred to herein as “pharmacometabolomics”). Pharmacometabolomics allows for the selection of agents (e.g., drugs) for therapeutic or prophylactic treatment of an individual based on the small molecule profile of the individual. The small molecule profile of the individual is examined to predict what the person's reaction to a particular therapeutic compound will be. Yet another aspect of the invention pertains to monitoring the influence of agents (e.g., drugs or other compounds) on the small molecule profiles of the patients in clinical trials.

Pharmacometabolomics is similar to pharmacogenomics but it is also able to taken in to account environmental and other non-genetic factors (e.g., other drugs, etc.) which may affect an individual's response to a particular therapeutic compound. Pharmacometabolomics can be used alone or in combination with pharmacogenomics to predict an individual's reaction to a particular drug based upon their small molecule profile and/or their genotype.

Pharmacometabolomics is particularly useful because it provides an early warning sign, due to its capability of detecting aberrant small molecules long before any disease symptoms or predisposed phenotypes are noticed.

Diagnostic Assays

In one embodiment, the invention pertains to a method for facilitating the diagnosis of a disease state of a subject. The method includes obtaining a small molecule profile from a subject suspected of having and/or having a disease state, and comparing the small molecule profile from the subject to a standard small molecule profile.

The invention provides a method of assessing small molecule profiles, especially aberrant small molecule profiles. Aberrant small molecule profiles (e.g., excessive amounts of a particular molecule, deficient amounts of a particular molecule, the presence of a small molecule not usually present, etc.) may indicate the presence of a disease state. More generally, aberrant small molecule profiles may indicate the occurrence of a deleterious or disease-associated profile contributed by small molecules present in aberrant amounts.

The standard small molecule profile can be obtained from healthy subjects or subjects afflicted with the disease state which the subject is suspected of having. The small molecule profiles can be taken from a particular organ, tissue, or combinations or organs or tissues, The small molecule profiles can also be taken of cells, cellular compartments, particular organelles, or extracellular material.

Prognostic Assays

The invention also pertains to a method for predicting whether a subject is predisposed to having a disease state. The method includes obtaining a small molecule profile from the subject; and comparing the small molecule profile from the subject to a standard small molecule profile, thereby predicting whether a subject is predisposed to having a disease state.

The methods described herein can furthermore be used as diagnostic or prognostic assays to identify subjects having or at risk of developing a disease or disorder associated with aberrant small molecule profiles. For example, the assays described herein, such as the preceding diagnostic assays or the following assays, can be utilized to identify a subject having or at risk of developing a disorder associated with an aberrant small molecule profile, such as drug resistance of tumor cells. Alternatively, the prognostic assays can be utilized to identify a subject having or at risk for developing such a disease or disorder. Thus, the present invention provides a method in which a test sample is obtained from a subject and a small molecule profile is taken, wherein an aberrant small molecule profile is diagnostic for a subject having or at risk of developing a disease or' disorder associated with an aberrant small molecule profile. The term “test sample” is a biological sample obtained from a subject of interest. For example, a test sample can be a biological fluid (e.g., serum, blood, saliva, etc.), cell sample, or tissue. Advantageously, the test sample may consist of cells, extracellular material, or individual organelles, e.g., mitochondria, nuclei, Golgi apparatus, endoplasmic reticulum, ribosomes, chloroplasts, etc.

Furthermore, the prognostic assays described herein can be used to determine whether a subject can be administered an agent (e.g., peptidomimetic, protein, peptide, nucleic acid, small molecule, or other drug candidate) to treat a disease or disorder associated with an aberrant small molecule profile. For example, such methods can be used to determine whether a subject can be effectively treated with a specific agent or class of agents (e.g., agents of a type which affect the small molecule profile in particular ways). Thus, the present invention provides methods for determining whether a subject can be effectively treated with an agent for a disorder' associated with an aberrant small molecule profile in which a test sample is obtained and an aberrant small molecule profile is detected (e.g., wherein the presence or relative quantity of particular relevant small molecules is diagnostic for a subject that can be administered the agent to treat a disorder associated with the aberrant small molecule profile). In some embodiments, the foregoing methods provide information useful in prognostication, staging and management of particular states that are characterized by altered small molecule profiles and thus by a particular metaboprint. The information more specifically assists the clinician in designing treatment regimes to eradicate such particular states from the body of an afflicted subject.

The methods of the invention can also be used to detect the presence or absence of relevant small molecules, thereby determining if a subject is at risk for a disorder associated with this relevant small molecule. For example, the presence or absence of relevant small molecules, may indicate whether the process of developing a disease state has been initiated or is likely to arise in the tested cells. In preferred embodiments, the methods include detecting the presence or absence of the relevant small molecule, in a sample of cells or extracellular material from the subject, the presence or absence of a disease state. Preferably the sample of cells or extracellular material is obtained from a body tissue suspected of comprising diseased cells. Thus, the present method provides information relevant to diagnosis of the presence of a disease state. In one embodiment, the sample of cells is comprised mainly of a particular cellular organelle, e.g., mitochondria, Golgi apparatus, nuclei, etc.

The methods described herein may be performed, for example, by utilizing pre-packaged diagnostic kits comprising at least one reagent for detecting a relevant small molecule, which may be conveniently used, e.g., in clinical settings to diagnose patients exhibiting symptoms or family history of a disease or illness involving a relevant small molecule.

Pharmacometabolomics

The invention also pertains to a method for predicting a subject's response to a therapeutic agent. The method includes obtaining a small molecule profile from the subject, and comparing the small molecule profile of the subject to a known standard established for the therapeutic agent as an indication of whether the subject would benefit from treatment with the therapeutic agent.

Agents, or modulators which alter levels of particular relevant small molecules, as identified by a screening assay described herein can be administered to individuals to treat (prophylactically or therapeutically) disorders associated with the relevant small molecules. In conjunction with such treatment, the pharmacometabolomics (i.e., the study of the relationship between an individual's small molecule profile and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacometabolomics of the individual permits the selection of effective agents (e,g., drugs) for prophylactic or therapeutic treatments based on a consideration of the individual's small molecule profile. Such pharmacometabolomics can further be used to determine appropriate dosages and therapeutic regimens. Accordingly, the small molecule profile of an individual can be determined to thereby select appropriate agent(s) for therapeutic or prophylactic treatment of the individual.

The known standard can be obtained from subjects who benefited from the agent, e.g., patients who were treated with the agent and were cured, maintained their health, or prevented or slowed the deterioration of health. The known standard can be taken from a particular tissue, organ. It can also be taken from any organelle, cell, or cellular compartment during any point during the beneficial treatment. It can be derived from a single patient or from an average of more than one patient who were treated successfully with the agent. In addition, the known standard can also be derived using other techniques.

Small Molecules Databases and Methods of Use

In one embodiment, the invention pertains to the creation of small molecule databases containing information regarding the metabolome of cells, cellular compartments, and organelles, e.g., cells, cellular compartments, and organelles in health, diseased, and altered states. The information regarding the small molecules of each cell, cellular compartment, or organelle can be found using the separation and analytical techniques described elsewhere in the application. The small molecule databases can include compounds derived from the same or different animal organs. For example, the small molecule databases can include compounds obtained from cells of specific organs such as a heart, brain, kidney, liver, done, blood, gastrointestinal tract, and/or muscle. In addition, the small molecule databases can include information regarding compounds obtained from individuals suffering from a particular disease state, e.g., cardiovascular diseases, neurodegenerative diseases, diabetes, obesity, immunological disorders, etc.

The databases can be made based on information obtained from the techniques described elsewhere in the application to determine the identity and presence of various small molecules in cells, cellular compartments, and organelles. The databases may include information regarding the compounds found, such as structure, molecular weight, amounts found in particular organelles in a particular state of health, and any other information that a person of skill in the art would consider relevant and useful to be contained in the database. For example, information regarding known biochemical pathways involving the particular compound may also be included as well as other such information,

In one embodiment, the databases of the invention contain information on the compounds of the metabolome of a particular organelle of a particular species in a particular state of health from a particular organ (e.g., one database may include compounds of the metabolome of the mitochondria of a healthy human heart), In other embodiments, the databases may include information regarding the metabolome of a variety of organelles (e.g., mitochondria; nuclei, Golgi apparatus, endoplasmic reticulum, ribosomes, cytosol, chloroplasts, etc.) or cells from a particular species from a particular organ in a particular state of health. In another embodiment, the databases may include information regarding either specific organelles or cells from a variety of tissues (e.g., fatty tissue, muscle tissue, nerve tissue, brain tissue, heart tissue, bone tissue, blood, connective tissue, retinal tissue, etc.) from an organism in a health or diseased stated (e.g., the tissue can be from an organism suffering from any disorder known to afflict it). Examples of disorders include neurological disorders, central nervous system disorders, metabolic disorders, cardiovascular disorders, immunological disorders, oncological disorders. In a further embodiment, a database may comprise information regarding compounds of the entire metabolome of a particular species, e.g., human.

If the database is in electronic form, the program used to organize the database can be any program known in the art which is capable of storing the information in a useful format.

The databases of the invention can be organized in such a way that they can be licensed to companies, such as pharmaceutical companies. The databases can then be used for many purposes, such as drug discovery, design, etc.

### Transcriptome

The gene expression of the genes related to conditions and/or diseases associated with Down syndrome according to the present invention is either up-regulated or down-regulated in individuals with Down syndrome having these conditions/diseases compared to typical individuals. By determining the level of gene expression through measuring of the amounts of DNA, RNA or gene expression products in cells from a Down syndrome sample and comparing the gene expression from the sample cells with the gene expression in control cells, it is possible to identify individuals with Down syndrome that will have these conditions and/or diseases.

In another embodiment, the gene expression of the genes related to conditions and/or diseases more prevalent in typical individuals according to the present invention is either up-regulated or down-regulated in typical individuals having these conditions/diseases compared to individuals with Down syndrome. By determining the level of gene expression through measuring of the amounts of DNA, RNA or gene expression products in cells from a typical sample and comparing the gene expression from the sample cells with the gene expression in control cells, it is possible to identify typical individuals that will have these conditions and/or diseases prevalent in typical individuals.

The present invention therefore provides a method for predicting or assessing the disease spectrum of an individual with Down syndrome, wherein the method comprises the steps of (a) determining the gene expression of genes in an individual with Down syndrome test sample and a control sample derived from a typical individual, wherein the gene expression is determined, and (b) determining whether the genes are up-regulated or down-regulated in the Down syndrome sample compared to the typical sample.

The present invention also provides a method for predicting or assessing the disease spectrum of a typical individual, wherein the method comprises the steps of (a) determining the gene expression of genes in a typical individual test sample and a control sample derived from an individual with Down syndrome, wherein the gene expression is determined, and (b) determining whether the genes are up-regulated or down-regulated in the typical sample compared to the Down syndrome sample.

The present invention furthermore relates to a kit for distinguishing between typical and Down syndrome cells by quantitative determination of mRNA, which comprises a solid support on which different isolated polynucleotides are immobilized.

The present invention also relates to the use of a compound for the manufacture of a medicament for the treatment of a condition and/or disease associated with Down syndrome, wherein the compound increases or decreases the expression of genes identified in a transcriptomics experiment. In an exemplary embodiment, the compound decreases the expression or activity of one or more proteins listed in Table 1 in an individual with Down syndrome. In another exemplary embodiment, the compound increases the expression or activity of one or more proteins listed in Table 2 in an individual with Down syndrome. In one embodiment, the protein in Table 1 or Table 2 is fibroblast growth factor receptor, epidermal growth factor receptor, platelet-derived growth factor receptor, neuropilin, matrix metalloproteinase, B2-microglobulin, Cystatin C, Annexin Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin. In another embodiment, the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1 C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE, In a further embodiment, the condition or disease associated with Down's syndrome comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

In another embodiment, the present invention relates to the use of a compound for the manufacture of a medicament for the treatment of a condition and/or disease prevalent in a typical individual, wherein the compound increases or decreases the expression of genes identified in a transcriptomics experiment. In an exemplary embodiment, the compound increases the expression or activity of one or more proteins listed in Table 1 in a typical individual, In another exemplary embodiment, the compound decreases the expression or activity of one or more proteins listed in Table 2 in a typical individual. In one embodiment, the protein in Table 1 or Table 2 is fibroblast growth factor receptor, epidermal growth factor receptor, platelet-derived growth factor receptor, neuropilin, matrix metalloproteinase, B2-microglobulin, Cystatin C, Annexin II, Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin. In another embodiment, the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch?), TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE. In a further embodiment, the condition or disease associated with a typical individual comprises heart disease, cancer, stroke, coronary artery disease, atherosclerosis, hypertension and/or angiopathies.

In an exemplary embodiment, the invention provides a method for treating a condition or disease prevalent in an individual with Down syndrome, the method comprising administering an effective amount of a pharmaceutical composition to the individual. The pharmaceutical composition administered reduces the expression or activity of a protein in Table 1 associated with the condition or disease prevalent in the individual with Down syndrome and/or increases the expression or activity of a protein in Table 2 associated with the condition or disease prevalent in the individual with Down syndrome. In one embodiment, the protein in Table 1 or Table 2 is fibroblast growth factor receptor, epidermal growth factor receptor, platelet-derived growth factor receptor, neuropilin, matrix metalloproteinase, B2-microglobulin, Cystatin C, Annexin II, Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin. In another embodiment, the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE. In a further embodiment, the condition or disease prevalent in an individual with Down's syndrome comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

In another exemplary embodiment, the invention provides a method for treating a condition or disease prevalent in a typical individual but rarely occurring in an individual with Down syndrome, the method comprising administering an effective amount of a pharmaceutical composition to the individual. The pharmaceutical composition administered increases the expression or activity of a protein in Table 1 and/or reduces the expression or activity of a protein in Table 2. In one embodiment, the protein in Table 1 or Table 2 is fibroblast growth factor receptor, epidermal growth factor receptor, platelet-derived growth factor receptor, neuropilin, matrix metalloproteinase, B2-microglobulin, Cystatin C, Annexin II, Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin. In another embodiment, the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE. In a further embodiment, the condition or disease prevalent in a typical individual but rarely occurring in an individual with Down syndrome comprises heart disease, cancer, stroke, coronary artery disease, atherosclerosis, hypertension and/or angiopathies.

In another embodiment, the invention provides a method for treating a condition or disease prevalent in an individual with Down syndrome comprising administering an effective amount of a pharmaceutical composition to the individual, wherein the expression or activity level of a protein in Table 1 is higher than a predetermined threshold level, and wherein the effective amount of the pharmaceutical composition reduces the expression or activity level of the protein in the individual. In another embodiment, the invention provides a method for treating a condition or disease prevalent in an individual with Down syndrome comprising administering an effective amount of a pharmaceutical composition to the individual, wherein the expression or activity level of a protein in Table 2 is lower than a predetermined threshold level, and wherein the effective amount of the pharmaceutical composition increases the expression or activity level of the protein in the individual. In one embodiment, the protein in Table 1 or Table 2 is fibroblast growth factor receptor, epidermal growth factor receptor, platelet-derived growth factor receptor, neuropilin, matrix metalloproteinase, B2-microglobulin, Cystatin C, Annexin II, Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin. In another embodiment, the at least one biomarker is FGFR1, EGFR, ERBB3, ERBB4, PDGFR, NRP1 MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1IS, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE. In a further embodiment, the condition or disease prevalent in an individual with Down's syndrome comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

In another embodiment, the invention provides a method for treating a condition or disease prevalent in a typical individual comprising administering an effective amount of a pharmaceutical composition to the individual, wherein the expression or activity level of a protein in Table 1 is lower than a predetermined threshold level, and wherein the effective amount of the pharmaceutical composition increases the expression or activity level of the protein in the individual. In another embodiment, the invention provides a method for treating a condition or disease prevalent in a typical individual comprising administering an effective amount of a pharmaceutical composition to the individual, wherein the expression or activity level of a protein in Table 2 is higher than a predetermined threshold level, and wherein the effective amount of the pharmaceutical composition decreases the expression or activity level of the protein in the individual. In one embodiment, the protein in Table 1 or Table 2 is fibroblast growth factor receptor, epidermal growth factor receptor, platelet-derived growth factor receptor, neuropilin, matrix metalloproteinase, B2-microglobulin, Cystatin C, Annexin II, Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin. In another embodiment, the at least one biomarker is FGFR1, EGFR, ERBB3, ERBB4, PDGFR, NRP1, MMPI , B2-microglobulin (B2M), Cystatin C (CST3), Annexin II (ANXA2), Notch1, Notch3, TNF sR-1 TNFRSF1A), TNF sR-2 (TNFRSFIB), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, CIS, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE. In a further embodiment, the condition or disease prevalent in a typical individual comprises heart disease, cancer, stroke, coronary artery disease, atherosclerosis, hypertension and/or angiopathies.

In a preferred embodiment of the invention, the gene expression is determined by measuring the mRNAs or gene expression products corresponding to the genes associated with Down syndrome in a quantitative manner.

It is preferred that the Down syndrome sample and the typical sample comprise monocytes.

In another preferred embodiment of the invention, the step of determining whether the genes are up-regulated or down-regulated in the test sample compared to the control sample comprises to determine the fold change values of the genes in the test sample. In one embodiment, the test sample is from an individual with Down syndrome. In another embodiment, the test sample is from a typical individual. In one embodiment the control sample is from a typical individual. In another embodiment, the control sample is from an individual with Down syndrome.

It is preferred that a gene is defined as up-regulated if its fold change value is at least 2 and down-regulated if its fold change value is smaller than or equal to 0,5.

It is further preferred that a gene is defined as up-regulated if its fold change value is at least 3 and down-regulated if its fold change value is smaller or equal to 0.33.

It is further preferred that the median false discovery rate (FDR) of the method for determining and analyzing the gene expression is smaller than 10%, more preferred smaller than 7% and even more preferred smaller than 4%,

Furthermore it is preferred that the step of determining the gene expression comprises the use of hybridization technology and/or polymerase chain reactions (PCR),

In one embodiment of the present invention the PCR method comprises: a) contacting the mixture of mRNAs or cDNAs from said sample with amplification reagents comprising pairs of primers, wherein said pairs of primers substantially correspond or are substantially complementary to the gene sequences of the genes to be determined, b) carrying out an amplification reaction, c) measuring the generation of amplification products; and d) determining the quantity of mRNA in said sample from the results obtained in step c).

It is preferred that said amplification reaction is a real-time-PCR (polymerase chain reaction).

In another embodiment, the hybridization technology comprises measuring the gene expression by hybridizing the mRNAs or cDNAs of the samples with complementary nucleotide probes immobilized on a solid support.

The present invention furthermore relates to a method for identifying compounds which modulate the expression of any of the genes associated with a condition and/or disease of Down syndrome or of a typical individual, comprising: (a) contacting a candidate compound with cells which express said genes and (b) determining the effect of said candidate compound on the expression of said genes.

In a preferred embodiment of the invention the candidate compound is selected from the group consisting of si-RNA, anti-sense-RNA or other interfering nucleic acids, antibodies, aptamers and small molecules.

It is preferred that the step of determining the effect of a compound on the gene expression according to this method, comprises comparing said expression with the expression of said genes in cells which were not contacted with the candidate compound.

In some embodiments, the effect of candidate compounds can first be screened in an animal model. For instance, the Ts65Dn mice—a model of Down syndrome—can be utilized to assess the effect of various candidate compounds on gene and/or protein expression. Ts65Dn mice are trisomic for about two-thirds of the genes orthologous to human chromosome 21 and are a well-characterized model for studying Down syndrome.

The present invention also relates to a kit for detecting a condition and/or disease associated with an individual with Down syndrome by quantitative determination of mRNA according to the method as described above. In another embodiment, the present invention also relates to a kit for detecting a condition and/or disease more prevalent in a typical individual by quantitative determination of mRNA according to the method as described above.

In a preferred embodiment, the kit comprises a solid support on which at least one isolated polynucleotide is immobilized.

The biological sample of the present invention can be any sample derived from or containing body liquid or tissue material such as e.g. samples of blood. It is preferred that the sample comprises plasma, red blood cells or white blood cells.

Usually, the sample has been processed to be in a condition suitable for the method of determining the gene expression. The processing may include dilution, concentration, homogenization, extraction, precipitation, fixation, washing and/or permeabilization, etc. The processing may also include reverse transcription according to methods well known in the field.

The phrase “determining the gene expression” as used herein preferably means “determining the expression level”. The expression or expression level correlates with the amount of polynucleotide or expression product thereof in the sample.

The gene expression can be determined by qualitatively or quantitatively measuring the function, protein level, mRNA level or the gene copy number referring to these genes. It is preferred to determine the gene expression quantitatively. The mRNA level determination can furthermore comprise to measure the amount of the corresponding cDNA, whereas the synthesis of cDNA can be performed applying common techniques. It is preferred to use cDNA in the method according to the present invention. The gene expression products or the total cellular RNA are isolated from these samples by techniques well known in the field. For example the single-step guanidinium-thiocyanate-phenol-chloroform method described in Chomczynski and Sacchi (Anal. Biochem. 1987; 162:156-159) can be used. The LiCl/urea method described in Auffray and Rougeon (Eur. J. Biochem. 1980; 107:303) can also be used.

The quantitative determination of the expression of the above described genes can be performed by measuring the amount of RNA, mRNA, genomic DNA (obtained by cloning or produced synthetically) or cDNA corresponding to said genes. The DNA may be double- or single-stranded. Single-stranded DNA may be the coding strand, also known as the sense strand, or it may be the noncoding strand, also referred to as the antisense strand. The quantitative determination of the gene expression can be performed by using the hybridization technology or a polymerase chain reaction or a mixture or a combination of these techniques, but is not limited to these methods. Furthermore the gene expression can be determined by measuring the amount of gene expression products (polypeptides) referring to these genes by common methods.

Assaying the gene copy number of the genes of the present invention can be performed by any known technique such as, for example, by visualizing extrachromosomal double minutes (dmin) or integrated homogeneously staining regions (hsrs) (Gebhart et al., Breast Cancer Res. Treat. 1986; 8:125; or Dutrillaux et al., Cancer Genet. Cytogenet. 1990; 49:203). Other techniques such as comparative genomic hybridization (CGH) single nucleotide polymorphism (SNP) and a strategy based on chromosome microdissection and fluorescence in situ hybridization can also be used to search for regions of increased DNA copy number in tumor cells (Guan et al., Nature Genet. 1994; 8:155).

The hybridization technology comprises contacting RNA, (such as mRNA) or DNA (such as cDNA) with a nucleotide probe. It is preferred that the nucleotide probes are immobilized on a solid support. The nucleotide sequence to be determined (target) hybridizes to the nucleotide probe, wherein a double-strand is formed. In order to measure the amount of hybridization products, common methods can be applied, such as spectroscopic techniques using fluorescent dyes.

The polynucleotide probes of the present invention, which can be freely dissolved or can be immobilized have a sequence according to the genes as described above or are complementary to these genes. A person skilled in the art is well aware that also characteristic fragments of these sequences are suitable for the detection of the targets. Furthermore probes may have a sequence which is a variant of the sequences of the genes of the present invention. The variant may be a sequence having one or more additions, substitutions, and/or deletions of one or more nucleotides such as an allelic variant or single nucleotide polymorphism of the sequences of the marker genes. It is preferred that the probes are at least 80% identical to the target gene sequences, whereas the probe can also have the complementary sequence. Further preferred is that the probes are at least 85%, more preferred 90% and even more preferred 97% identical or complementary to the target gene sequences.

The fluorescent dyes to be used for the spectroscopical quantification can be directly attached to the nucleotide probe, but can also be present in solution without any covalent-bond to the nucleotide. The use of oligonucleotide probes comprising at least one intercalator pseudonucleotide is disclosed in US 2006/0014144 and is incorporated by reference. The intercalation of the fluorescent dye into the double-stranded hybrid results in specific fluorescent properties, which can be measured by using common methods. Suitable assay formats for detecting hybrids formed between probes and target nucleic acid sequences in a sample are known in the art and include the immobilized target assay formats, such as the dot-blot format, and immobilized probe assay formats, such as the reverse dot-blot assay. Dot blot and reverse dot blot assay formats are described in U.S. Pat. Nos. 5,310,893; 5,451,512; and 5,468,613. The northern blot method among others is e.g. disclosed in U.S. Pat. No. 5,981,218 and furthermore in Harada et. al (Cell 1990; 63:303-312), whereas all methods therein are incorporated by reference.

However, any known standard hybridization technique can be used according to the invention. One preferred example, using immobilized nucleotide probes is the microarray technology. The microarray technology, which is also known as DNA chip technology, gene chip technology and solid-phase nucleic acid array technology, is well known to the skilled person and is based on, but not limited to, obtaining an array of identified nucleic acid probes on a fixed support, labelling target molecules with reporter-molecules (e.g., radioactive, chemiluminescent or fluorescent tags), hybridizing target nucleic acids to the probes, and evaluating target-probe hybridization. A probe with a nucleic acid sequence that perfectly matches the target sequence will, in general, result in detection of a stronger reporter-molecule signal than with probes with less perfect matches. Many components and techniques utilized in nucleic acid microarray technology are presented in “The Chipping Forecast”, Nature Genetics, volume 21, January 1999.

According to the present invention, microarray supports may include but are not limited to glass, silica, aluminosilicates, borosilicates, plastics, metal oxide, nitrocellulose or nylon. The use of a glass support is preferred. According to the invention, probes are selected from the group of polynucleotides including, but not limited to DNA, genomic DNA, cDNA and oligonucleotides; and maybe natural or synthetic. Oligonucleotide probes preferably are 20-25-mer oligonucleotides and DNA/cDNA probes preferably are 500-5000 bases in length, although other lengths may be used. Appropriate probe length may be determined by the skilled person by known procedures. Probes may be purified to remove contaminants using standard methods known to those of ordinary skill in the art such as gel filtration or precipitation. Accordingly, the polynucleotide immobilized to the solid support is preferably an isolated polynucleotide. The term “isolated” polynucleotide refers to a polynucleotide that is substantially free from other nucleic acid sequences, such as and not limited to other chromosomal and extrachromosomal DNA and RNA. Isolated polynucleotides may be purified from a host cell. Conventional nucleic acid purification methods known to skilled artisans may be used to obtain isolated polynucleotides. The term also includes recombinant polynucleotides and chemically synthesized polynucleotides.

In one embodiment, probes are synthesized directly on the support in a predetermined grid pattern using methods such as light-directed chemical syntheses, photochemical deprotection or delivery of nucleotide precursors to the support and subsequent probe production. In embodiments of the invention one or more control polynucleotides are attached to the support. Control polynucleotides may include but are not limited to cDNA of genes such as housekeeping genes or fragments thereof.

The solid support comprises at least one polynucleotide immobilized on or attached to its surface, wherein said polynucleotide hybridizes with a polynucleotide as described supra, preferably under stringent conditions. Suitable hybridization conditions are for example described in the manufacturers instructions of “DIG Easy HYB Granules” (Roche Diagnostics GmbH, Germany, Cat. No. 1796895). These instructions are incorporated herein by reference. The hybridization conditions described in the following protocol may be used: 1. Hybridizations are carried out using DIG Easy Hyb buffer (Roche Diagnostics, Cat. No. 1796895). 2. Ten microliters of hybridization solution with probe is placed on the microarray and a cover slip carefully applied. 3. The slide is replaced in a hybridization chamber and incubated for 16 hours incubation at 42° C. 4. The cover slips are removed in a container with 2×SSC+0.1% SDS and the microarrays are washed for 15 minutes in 2×SSC+0.1% SDS at 42° C., followed by a 5 minutes wash in 0.1×SSC+0.1% SDS at 25° C., followed by two short washes in 0.1×SSC and 0.01×SSC at 25° C., respectively.

5. The microarrays are dried by centrifugation and can be stored at 4° C.

The detection of the fluorescence of the samples can be performed by any techniques known in the art and can also be performed analog to the methods as described for the PCR below. The quantitative determination of the target sequences is also well known in the art.

In one embodiment, preferred probes are sets of ten or more of the nucleic acid molecules as defined. In a specific embodiment, at least twenty different isolated polynucleotides are immobilized on said solid support.

In another embodiment, at least ten or at least 15, further preferred at least 20, further preferred at least 30, or at least 40 different isolated polynucleotides corresponding to or being complementary to the up-regulated and down-regulated genes as specified above are immobilized on said solid support, whereas preferably the solid support can additionally contain polynucleotides which do not refer to the genes of interest.

In another embodiment, the method comprises utilizing an antibody directed against a polypeptide encoded by the genes described above, e.g., an directed antibody against a protein listed in Table 1 in an individual with Down syndrome or an antibody directed against a protein listed in Table 2 in a typical individual. The antibody may be polyclonal or monoclonal, with monoclonal antibodies being preferred. The antibody is preferably immunospecific for anyone of the polypeptides encoded by the above genes. The antibodies can be used to detect a polypeptide by any standard immunoessay technique including a ELISA, flow cytometry, immunohistochemistry, immunoblotting, (western blotting), immunoprecipitation, BIACORE technology and the like, as will be appreciated by one of ordinary skill in the art.

Another possibility to perform the determination of the gene expression is the polymerase chain reaction (PCR). The PCR method can be used to amplify the above indicated RNA/mRNA or DNA/cDNA samples and allowed to quantitatively back-calculate the quantity and the concentration of the amount of specific polynucleotide (target) in the sample. The PCR method is well known in the art and for example disclosed in WO 99/28500 or Sambrook et al. (Molecular Cloning—A Laboratory Manual, Cold Spring Harbor Laboratory, Cold Spring Harbor, N.Y. 1989) or Nucleic Acid Hybridization (Hames and Higgins eds., 1984) or Current Protocols in Human Genetics (Dracopoli et al., eds, 1984 with quarterly updates, John Wiley & Sons, Inc.), all of which are incorporated herein by reference.

The PCR method utilizes a pair of oligonucleotides (primers), each hybridizing to one strand of a double-stranded DNA/RNA target. The target corresponds to the specific DNA/RNA, which has to be determined in a quantitative manner. The primers flank the region that will be amplified. The PCR method comprises contacting the primers and target sequence or mixture of target sequences and optional polynucleotide probes and performing the amplification steps.

The primer will contain a “hybridizing region” exactly or substantially complementary or corresponding to a nucleotide sequence (target sequence) from one gene which is related to a condition and/or disease associated with Down syndrome or prevalent in a typical individual. The amplification is carried out using the primer, whereas the primer extension is performed under sufficiently stringent hybridization conditions, which allow the selective amplification of specific target sequences. Preferably the primer is from 15 to 35 nucleotides in length. A primer can either consist entirely of the hybridizing region or can contain additional features which allow for detection, immobilization, or manipulation of the amplified product, but which do not alter the basic property of the primer (acting as a point of initiation of DNA synthesis).

The primers can furthermore comprise covalently-bound fluorescent dyes, which confer specific fluorescence properties to the hybrid consisting of the primer and the target-sequence. An example for this method is the LUX™-primer technique (GEN et al., J Virol Methods 2004; 22:57-61) utilizing specific fluorescent dyes, which change their fluorescence properties because of the structural changes occurring due to the formation of the double-stranded DNA/RNA.

But it is also possible to use fluorescent dyes which are not covalently-bound to the primer, but can interact with the double-stranded DNA/RNA to change the fluorescence properties, Fluorescent dyes which can be used are for example SYBR-green or Ethidium bromide (U.S. Pat. No, 6,346,386 or Zipper et al., Nucl. Acid Res. 2004; 32;103),

If amplification products have to be determined by fluorescence and a mixture of target sequences is amplified at the same time in the same reaction mixture, the different targets have to be specifically and separately detected. This can be done by using primers comprising fluorescent dyes, whereas the different primers can be detected at different wavelength, due to different fluorescent properties. Different fluorescence properties can be achieved with different dyes or additional covalently attached dyes which alter the fluorescence properties by changing the electronic properties. But also any other method can be used to influence the fluorescence properties.

The detection of the fluorescence of the samples can be performed by any techniques known in the art. For example UV/Vis spectrophotometers can be used to determine the intensity of the signals at different wavelengths.

Another possibility is to use polynucleotide probes in addition to the pairs of primers. These probes contain a “hybridizing region” which is exactly or substantially complementary to the target sequence and specifically hybridizes to one amplified target sequence. Furthermore these probes comprise fluorescent dyes, Again the fluorescence properties of these probes are different and allow the detection of each target separately. This method has for example been reported by Wong et al (BioTechniques 2005; 39:75-85) and EP0678581. Also other techniques can be used such as: Taq Man, Molecular Beacon, ARMS, Scorpions, FRET (DE19755642 and Tyagi et al., Nat. Biotechnol. 2000; 18:1191-1196) etc. These methods are well known in the art and can be adapted and performed by a person skilled in the art. Of course, the PCR methods which can be used according to the present invention are not limited to these examples.

For the polynucleotide probes suitable for the PCR method the same applies as for the probes as specified for the hybridization technique above.

A person skilled in the field is able to synthesize suitable primers according to common techniques, based on the polynucleotide-sequences of the genes as identified above. It is understood that the primers can be suitable to amplify the complete corresponding or complementary nucleotide sequence of the marker genes or can be suitable to amplify only a part of this sequence, whereas the primers are selected to be suitable to specifically amplify and identify one marker gene. Accordingly pairs of primers have to be selected for every marker gene which has to be determined.

The hybridized primer acts as a substrate for a thermostable DNA polymerase (most commonly derived from Thermus aquaticus and called Taq polymerase) that synthesizes a complementary strand via a sequential addition of deoxyribonucleotides. The process includes repetitive cycles of three steps, denaturation of double-stranded DNA, annealing of the primers and extension of the DNA fragments, which are accomplished by cycle temperature changes in the reaction. The number of repetitive cycles varies usually from 25 to 50 in PCR tests used for diagnostic purposes. If the starting material is RNA/mRNA, a further step with a reverse transcriptase (RD enzyme can be performed before amplification. This technique is then referred to as RT-PCR, which is e.g. described by Makino et al, (Technique 1990; 2:295-301) or WO 97/06256.

Quantitation of a sample containing an unknown number of target sequences typically is carried out with reference to a “standard curve” generated from a series of amplifications of samples containing the target sequence in a range of known amounts. The standard curve is used to calculate an input copy number from the signal generated during an amplification. Thus, the unknown target sequence copy number in the sample of interest is estimated using the standard curve by calculating the copy number that previously was determined to yield a signal equal to that observed. The concentration of the target sequence in the sample then is calculated from the input copy number and the sample size, which is determined prior to reaction.

Quantitative estimates can be sensitive to variability in either the input sample size or in the reaction efficiency. The effect of inter-reaction viability of the input sample size on the calculated target concentration can be eliminated by using a control gene. A control gene provides an independent measure of the amount of RNA in the sample. The calculated concentration of target mRNA is adjusted based on the independent measure of sample size.

It is especially preferred to use a real-time PCR, whereas the accumulation of the PCR products is monitored continuously during the PCR run. There are several instruments available for real-time PCR, in which the accumulation of the product is monitored by measuring the fluorescence in each cycle, and these methods can be used according to the invention. The measured fluorescence is plotted against the cycle number. The cycle number, in which the exponential amplification (threshold cycle CT) is first detected over background, has an inverse linear relationship to the amount of target in the initial reaction. Absolute quantitation of the amount of target in the initial sample can be accomplished by measuring its CT value and using the external standard curve to determine the target sequence, By using the real time FOR it is possible to determine quantitatively the amount of mRNA in a sample. If the concentration of a mRNA species in a sample is low, additional cycles are required in order to detect a signal compared to a higher concentration of the mRNA species.

The real-time FOR can also be combined with the microarray technique which allows to quantitatively and simultaneously determine a plurality of nucleic acids. This method is disclosed in US 2006/0088844 and incorporated by reference.

In a special embodiment, pairs of primers are selected, which are suitable to each amplify a specific nucleotide sequence which can be found in more than one marker gene, whereas in this case every pair of primers amplifies parts of the DNA/RNA sequences corresponding to more than one marker gene. In other words, during the gene expression determination more than one gene is subject to the same pair of primers and can be amplified by this primer pair. The amount of DNA/RNA measured for this subgroup of genes corresponds to the total amount of gene expression for this subgroup by addition of the single levels of gene expression of every member of the subgroup, This allows a comparison of this gene expression value of the subgroup with the control sample derived from a healthy individual. In this case it is not possible to give the gene expression value for the single members of this subgroup, but only the total value.

After the amount of polynucleotide referring to the genes according to the invention has been determined, a comparison has to be made with the values observed in healthy cells, Amplification-based quantitation methods using an internal standard are described in U.S. Pat, Nos. 5,219.727 and 5,476,774, incorporated herein by reference. In these methods the internal standard is added to the reaction in a known copy number and co-amplified along with the RNA/DNA target.

It is furthermore possible to use a probe-less method, referred to herein as a kinetic-FOR method, for measuring the increase in amplified nucleic acid by monitoring the increase in the total amount of double-stranded DNA in the reaction mixture. This method is described in Higucci et al, (Bio/Technology 1992; 10:413-417; Bio/Technology 1993; 11:1026-1030) and U.S. Pat. No. 5,994,056, EP 487,218 and EP 512,334, each incorporated herein by reference. The detection of double stranded target DNA can be performed by using fluorescent dyes as described above.

It is also possible to measure the level of gene expression by determining the amounts of gene expression products in the samples. For example antibody-based methods are useful for detecting the gene expression and include immunoassays, such as the enzyme linked immunosorbent assay (ELISA) and the radio immunoassay (RIA), For example, a monoclonal antibody can be used both as an immunoabsorbent and as an enzyme-labelled probe to detect and quantify the proteins, corresponding to the genes which are either upregulated or downregulated in Down syndrome or in typical individuals. These methods can be performed according to standard procedures, whereas also any other method known in the art can be used to measure the amount of gene expression products in the samples. For example such an ELISA for detecting a tumor antigen is described in lacobelli et al. (Breast cancer research and treatment 1988; 11:19-30).

For determining whether or not a sample refers to an individual having a condition and/or disease, the gene expression level of the different marker genes has to be compared to the expression level in reference samples derived from healthy individuals. As will be appreciated in the art, once a standard expression level is known, it can be used repeatedly as a standard for comparison. Furthermore samples obtained from individuals having a condition and/or disease can be used for comparison. In this case, if the test sample and the sample for comparison have been obtained from individuals suffering from a condition and/or disease, a relative prognosis between these individuals can be provided.

The quantification of the gene expression in the sample for comparison can be performed using the same method as for the test sample or any other method which is known in the art. It is preferred that the same method is used for the test sample and the samples for comparison. The fold change value is calculated as the quotient of probe versus control samples. If the gene is up-regulated, a fold change value .gtoreq.two is regarded as significant, If the gene is down-regulated, a fold change value .ltoreq.0.5 is regarded as significant.

The comparison between the test sample and the control sample allows the identification of differentially expressed genes. The analysis of the expression patterns in these samples reveal which specific genes are up-regulated or down-regulated or normally expressed in the sample. The analysis leads to an arbitrary expression value based on specific hybridization intensity for the genes associated with a condition and/or disease in an individual with Down syndrome or typical individual, wherein the fold change value indicates if a gene is down-regulated or up-regulated or normally expressed. A gene is identified as up-regulated, if the fold change (FC) is greater than 2, preferably greater than 3. A gene is identified as down-regulated if the fold change (FC) is smaller than 0.5, preferably smaller than 0.33. In addition it is preferred to determine the median false discovery rate (FDR) of the method for determining and analyzing the gene expression, whereas it is preferred that the method displays a median false discovery rate of smaller than 10%, more preferred smaller than 7% and even more preferred smaller than 4%.

In order to determine whether a sample is from an individual having a condition and/or disease, the number of genes which are up-regulated or down-regulated have to be compared to the number of total genes which have been measured in the individual. In order to make a reliable diagnosis, it is preferred that at least 40% of these genes are identified as up- or down-regulated. It is even more preferred that at least 60% and further preferred at least 80% of these genes are up- or down-regulated. For example if at least 83% of 40 genes are up-regulated or down-regulated the estimated accuracy is .gtoreq.98%. It is preferred to perform the diagnosis of a sample by using the PAM method. If at least 90° k of 42 genes are up-regulated or down-regulated the estimated accuracy is .gtoreq.92%.

Another aspect of this invention is a method for identifying compounds which modulate the expression of the genes associated with a condition and/or disease in an individual with Down syndrome or typical individual. This method comprises contacting compounds with cells obtained from patients suffering from the condition and/or disease. A comparison between the gene expression in the presence of these compounds and without these compounds allows the identification of compounds which can be used to modulate the expression of these genes. The candidate compound may be selected if the expression of said genes in the cells which were contacted with the candidate compound is lower than in the cells which were not contacted with the candidate compound. In such case, the compound is capable of suppressing the expression of the genes referring to the disease. One may further compare the viability of the cells which were contacted with the candidate compound and the viability of cells which were not contacted with the candidate compound.

For example the antisense-, siRNA, antibodies, aptamers, anticalins and other small molecules designed to the genes and their products which are correlated with a condition and/or disease in an individual with Down syndrome or typical individual according to the invention can be used to modulate the gene expression.

The compounds which can modulate gene expression can be used for the treatment of a condition and/or disease in an individual with Down syndrome or typical individual.

Another aspect of the present invention is to provide a method for monitoring the progress of a condition and/or disease in an individual with Down syndrome or typical individual. By determining the gene expression levels at different points of time in the therapy, it is possible to draw conclusions, whether a therapy shows an effect and the sample displays a level of gene expression which is smaller than at the beginning of a therapy.

The present invention furthermore provides a kit for carrying out the method for diagnosis of a condition and/or disease in an individual with Down syndrome or typical individual according to the present invention.

Additionally the kit comprises primers suitable for the detection of the individual genes, It is preferred that one pair of primers is suitable to determine more than one gene, further preferred more than three genes. The kit also comprises the reagents necessary for carrying out the PCR reaction and quantitative measuring the amounts of target sequences. The kit further comprises the Taq polymerase. Additionally the kit comprises suitable nucleotide probes for selective quantitative determination of the different gene levels in the sample.

### Microbiome

The human intestine hosts up to 1014 bacteria, which harmoniously balance the immune system, help digest food, produce vitamins, and promote gastrointestinal (GI) motility. Hence, loss of homeostasis in the gut may contribute to the imbalance of disease states. The present embodiments provide for the characterization of one or more typical gut microbiomes and for the characterization of one or more gut microbiomes from subjects with Down syndrome. One potentially important environmental factor in disease states is abnormal intestinal flora, which often interacts with other factors such as intestinal permeability and transport of toxic substances, Some subjects with Down syndrome have autism and intestinal atresia. Considering the interactions of intestinal microflora and the central nervous system, human intestinal microbes might also contribute to the autistic symptoms regardless of the manifestation as GI problems.

Techniques for characterizing the microbiome include use of nucleic acid and/or proteins. Nucleic acid analysis includes analysis of , for example, DNA, RNA, mRNA, rRNA, and/or tRNA, and can be accomplished using, for example, pyrosequencing, qPCR, RT-qPCR, clone libraries, DGGE, T-RFLP, ARISA, microarrays, FIFH, dot-blot hybridization, next generation sequencing, and any other DNA hybridization methods that will detect a specific sequence. Protein analysis includes, for example, 2-Dimensional Gel Electrophoresis, 2-Dimensional Difference Gel Electrophoresis (2D-DIGE), MALDI TOF-MS, (2D-) LC-ESI-MS/MS, AQUA, and iTRAQ. These characterizations can be combined with rigorous statistical analysis to determine the constituents of the microbiome. In one non-limiting example, parallel pyrosequencing, provides for high-capacity, low-cost sequencing. The present disclosure uses different statistical tests and the use of rigorous correction methods for multiple testing that strengthen the interpretation of the present data. Bioinformatics provides for the efficient definition of the characteristics and distributions of intestinal microflora between subjects.

Many gram-negative bacteria work as pathogens because their cell wall contains lipopolysaccharide (LPS), which stimulates host immune systems to cause fever and neurological dysfunction. LPS can increase the permeability of the blood-brain barrier and increase mercury levels in the cerebrum. LPS also tends to decrease levels of glutathione, an important antioxidant involved in heavy metal detoxification, Lower levels of glutathione may increase the vulnerability of children to autism and other neurologic disorders such as Parkinson's and Alzheimer's diseases,

The human intestine also possesses numerous protective commensal microbes. Microbes domesticate the host and tend to survive together in the long run. Bifidobacterium and Lactobacillus are good examples of beneficial bacteria in the human intestine, and are often used as probiotics to promote motility. Many Clostridium species are pathogenic, but it has been reported that the sub-group of Clostridium IV/XlVa have a beneficial role in maintaining a balanced immune system, similar to the segmented filamentous bacteria.

Molecular techniques such as those based on parallel sequencing enable thorough and systematic identification of intestinal microorganisms. From this, alterations in gut microbe composition can be linked to various human disorders.

The present invention also includes altering various combinations of species, such as at least two species, at least three species, at least four species, at least five species, at least six species, at least seven species, at least eight species, at least nine species, or at least ten species.

It is contemplated that the abundance of gut microorganisms within an individual subject may be altered (i.e., increased or decreased) from about a one fold difference to about a ten fold difference or more, depending on the desired result and the individual subject. In one embodiment, the abundance may be altered from about a one fold difference to about a ten fold difference. In another embodiment, the abundance may be altered by an increase of about a two fold difference to about a ten fold difference, of about a three fold difference to about a ten fold difference, of about a four fold difference to about a ten fold difference, of about a five fold difference to about a ten fold difference, or of about a six fold difference to about a ten fold difference.

Stated another way, it is contemplated that the abundance of gut microorganisms within an individual subject may be altered (i.e., increased or decreased) from about 1% to about 100% or more depending on the desired result and the individual subject. In one embodiment, the abundance may be altered by an increase of from about 20% to about 100%, from about 30% to about 100%, from about 40% to about 100%, from about 50% to about 100%, from about 60% to about 100%, from about 70% to about 100%, from about 80% to about 100%, or from about 90% to 100%.

Another aspect of the invention encompasses use of the gut microbiome as a biomarker for a condition or disease in an individual with Down syndrome or typical individual. The biomarker may be utilized to construct arrays that may be used for several applications including as a diagnostic or prognostic tool to determine disease risk judging efficacy of existing treatments, drug discovery, for the identification of additional biomarkers involved in the condition or disease, and for the discovery of therapeutic targets. Generally speaking, the array may comprise biomolecules from a diseased host microbiome or a healthy host microbiome.

Array

The array may be comprised of a substrate having disposed thereon at least one biomolecule that is modulated in a diseased host microbiome compared to a healthy host microbiome. Several substrates suitable for the construction of arrays are known in the art, and one skilled in the art will appreciate that other substrates may become available as the art progresses. The substrate may be a material that may be modified to contain discrete individual sites appropriate for the attachment or association of the biomolecules and is amenable to at least one detection method. Non-limiting examples of substrate materials include glass, modified or functionalized glass, plastics (including acrylics, polystyrene and copolymers of styrene and other materials, polypropylene, polyethylene, polybutylene, polyurethanes, TeflonJ, etc.), nylon or nitrocellulose, polysaccharides, nylon, resins, silica or silica-based materials including silicon and modified silicon, carbon, metals, inorganic glasses and plastics. In an exemplary embodiment, the substrates may allow optical detection without appreciably fluorescing.

A substrate may be planar, a substrate may be a well, i.e. a 364 well plate, or alternatively, a substrate may be a bead. Additionally, the substrate may be the inner surface of a tube for flow-through sample analysis to minimize sample volume, Similarly, the substrate may be flexible, such as a flexible foam, including closed cell foams made of particular plastics.

The biomolecule or biomolecules may be attached to the substrate in a wide variety of ways, as will be appreciated by those in the art. The biomolecule may either be synthesized first, with subsequent attachment to the substrate, or may be directly synthesized on the substrate. The substrate and the biomolecule may be derivatized with chemical functional groups for subsequent attachment of the two. For example, the substrate may be derivatized with a chemical functional group including, but not limited to, amino groups, carboxyl groups, oxo groups or thiol groups. Using these functional groups, the biomolecule may be attached using functional groups on the biomolecule either directly or indirectly using linkers.

The biomolecule may also be attached to the substrate non-covalently. For example, a biotinylated biomolecule can be prepared, which may bind to surfaces covalently coated with streptavidin, resulting in attachment. Alternatively, a biomolecule or biomolecules may be synthesized on the surface using techniques such as photopolymerization and photolithography. Additional methods of attaching biomolecules to arrays and methods of synthesizing biomolecules on substrates are well known in the art, i.e. VLSIPS technology from Affymetrix (e,g., see U.S. Pat. No. 6,566,495, and Rockett and Dix, “DNA arrays: technology, options and toxicological applications,” Xenobiotica 30(2):155-177, all of which are hereby incorporated by reference in their entirety).

In one embodiment, the biomolecule or biomolecules attached to the substrate are located at a spatially defined address of the array. Arrays may comprise from about 1 to about several hundred thousand addresses. In one embodiment, the array may be comprised of less than 10,000 addresses. In another alternative embodiment, the array may be comprised of at least 10,000 addresses. In yet another alternative embodiment, the array may be comprised of less than 5,000 addresses. In still another alternative embodiment, the array may be comprised of at least 5,000 addresses. In a further embodiment, the array may be comprised of less than 500 addresses. In yet a further embodiment, the array may be comprised of at least 500 addresses.

A biomolecule may be represented more than once on a given array. In other words, more than one address of an array may be comprised of the same biomolecule, In some embodiments, two, three, or more than three addresses of the array may be comprised of the same biomolecule. In certain embodiments, the array may comprise control biomolecules and/or control addresses. The controls may be internal controls, positive controls, negative controls, or background controls.

The array may be comprised of biomolecules indicative of a diseased host microbiome. Alternatively, the array may be comprised of biomolecules indicative of a healthy host microbiome. A biomolecule is “indicative” of a diseased or healthy microbiome if it tends to appear more often in one type of microbiome compared to the other. Additionally, the array may be comprised of biomolecules that are modulated in the diseased host microbiome compared to the healthy host microbiome. As used herein, “modulated” may refer to a biomolecule whose representation or activity is different in a diseased host microbiome compared to a healthy host microbiome. For instance, modulated may refer to a biomolecule that is enriched, depleted, up-regulated, down-regulated, degraded, or stabilized in the diseased host microbiome compared to a healthy host microbiome. In one embodiment, the array may be comprised of a biomolecule enriched in the diseased host microbiome compared to the healthy host microbiome. In another embodiment, the array may be comprised of a biomolecule depleted in the diseased host microbiome compared to the healthy host microbiome. In yet another embodiment, the array may be comprised of a biomolecule up-regulated in the diseased host microbiome compared to the healthy host microbiome. In still another embodiment, the array may be comprised of a biomolecule down-regulated in the diseased host microbiome compared to the healthy host microbiome. In still yet another embodiment, the array may be comprised of a biomolecule degraded in the diseased host microbiome compared to the healthy host microbiome. In an alternative embodiment, the array may be comprised of a biomolecule stabilized in the disease host microbiome compared to the healthy host microbiome.

Generally speaking, an array of the invention may comprise at least one biomolecule indicative or, or modulated in, a diseased host microbiome compared to a healthy host microbiome. In one embodiment, the array may comprise at least 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 biomolecules indicative of, or modulated in, a diseased host microbiome compared to a healthy host microbiome. In another embodiment, the array may comprise at least 200, at least 300, at least 400, at least 500, or at least 600 biomolecules indicative of, or modulated in, a diseased host microbiome compared to a healthy host microbiome.

As used herein, “biomolecule” may refer to a nucleic acid, an oligonucleic acid, an amino acid, a peptide, a polypeptide, a protein, a lipid, a metabolite, or a fragment thereof. Nucleic acids may include RNA, DNA, and naturally occurring or synthetically created derivatives. A biomolecule may be present in, produced by, or modified by a microorganism within the gut.

Biomolecules that are enriched in the diseased microbiome compared to the healthy microbiome may include biomolecules derived from the following Kyoto Encyclopedia of Genes and Genomes (KEGG) Categories: Carbohydrate Metabolism, Amino Acid Metabolism, Metabolism of Other Amino Acids, Glycan Biosynthesis and Metabolism, Biosynthesis of Polyketides and Nonribosomal Peptides, Transcription, Folding/Sorting/Degradation, Signal Transduction, and Cell Growth and Death. In certain embodiments, the biomolecules derived from the KEGG categories above may include biomolecules from a corresponding KEGG pathway.

Additionally, the biomolecule may be at least 70, 75, 80, 85, 90, or 95% homologous to a biomolecule having an accession number. In one embodiment, the biomolecule may be at least 80, 81, 82, 83, 84, 85, 86, 87, 88, or 89% homologous to a biomolecule having an accession number. In another embodiment, the biomolecule may be at least 90, 91, 92, 93, 94, 95, 96, 97, 98, or 99% homologous to a biomolecule having an accession number.

In determining whether a biomolecule is substantially homologous or shares a certain percentage of sequence identity with a sequence of the invention, sequence similarity may be determined by conventional algorithms, which typically allow introduction of a small number of gaps in order to achieve the best fit, In particular, “percent identity” of two polypeptides or two nucleic acid sequences is determined using the algorithm of Karlin and Altschul (Proc, Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See www.ncbi.nlm,nih.gov for more details.

The arrays may be utilized in several suitable applications. For example, the arrays may be used in methods for detecting association between two or more biomolecules. This method typically comprises incubating a sample with the array under conditions such that the biomolecules comprising the sample may associate with the biomolecules attached to the array. The association is then detected, using means commonly known in the art, such as fluorescence, “Association,” as used in this context, may refer to hybridization, covalent binding, or ionic binding, A skilled artisan will appreciate that conditions under which association may occur will vary depending on the biomolecules, the substrate, and the detection method utilized. As such, suitable conditions may have to be optimized for each individual array created.

In yet another embodiment, the array may be used as a tool in a method to determine whether a compound has efficacy for treatment of a condition and/or disease in a host.

The array may also be used to quantitate the plurality of biomolecules of the host microbiome before and after administration of a compound. The abundance of each biomolecule in the plurality may then be compared to determine if there is a decrease in the abundance of biomolecules associated with the condition andior disease after treatment.

In some embodiments, the array may be used as a diagnostic or prognostic tool to identify subjects that are susceptible to a condition and/or disease. Such a method may generally comprise incubating the array with biomolecules derived from the subject's gut microbiome to determine the relative abundance of microorganisms,

Microbiome Profiles

The present invention also encompasses use of the microbiome as a biomarker to construct microbiome profiles. Generally speaking, a microbiome profile is comprised of a plurality of values with each value representing the abundance of a microbiome biomolecule. The abundance of a microbiome biomolecule may be determined, for instance, by sequencing the nucleic acids of the microbiome. This sequencing data may then be analyzed by known software to determine the abundance of a microbiome biomolecule in the analyzed sample. The abundance of a microbiome biomolecule may also be determined using an array described above. For instance, by detecting the association between a biomolecule comprising a microbiome sample and the biomolecules comprising the array, the abundance of a microbiome biomolecule in the sample may be determined.

A profile may be digitally-encoded on a computer-readable medium. The term “computer-readable medium” as used herein refers to any medium that participates in providing instructions to a processor for execution. Such a medium may take many forms, including but not limited to non-volatile media, volatile media, and transmission media. Non-volatile media may include, for example, optical or magnetic disks. Volatile media may include dynamic memory. Transmission media may include coaxial cables, copper wire and fiber optics. Transmission media may also take the form of acoustic, optical, or electromagnetic waves, such as those generated during radio frequency (RF) and infrared (IR) data communications. Common forms of computer-readable media include, for example, a floppy disk, a flexible disk, hard disk, magnetic tape, or other magnetic medium, a CD-ROM, CDRW, DVD, or other optical medium, punch cards, paper tape, optical mark sheets, or other physical medium with patterns of holes or other optically recognizable indicia, a RAM, a PROM, and EPROM, a FLASH-EPROM, or other memory chip or cartridge, a carrier wave, or other medium from which a computer can read.

A particular profile may be coupled with additional data about that profile on a computer readable medium. For instance, a profile may be coupled with data about what therapeutics, compounds, or drugs may be efficacious for that profile. Conversely, a profile may be coupled with data about what therapeutics, compounds, or drugs may not be efficacious for that profile. Alternatively, a profile may be coupled with known risks associated with that profile. Non-limiting examples of the type of risks that might be coupled with a profile include disease or disorder risks associated with a profile. The computer readable medium may also comprise a database of at least two distinct profiles.

Such a profile may be used, for instance, in a method of selecting a compound for treating a condition and/or disease in a host. Generally speaking, such a method would comprise providing a microbiome profile from the host and providing a plurality of reference microbiome profiles, each associated with a compound, and selecting the reference profile most similar to the host microbiome profile, to thereby select a compound for treating a condition and/or disease in the host. The host profile and each reference profile may comprise a plurality of values, each value representing the abundance of a microbiome biomolecule.

The microbiome profiles may be utilized in a variety of applications. For example, the microbiome profiles may be used in a method for predicting risk for a condition and/or disease in a host. The method comprises, in part, providing a microbiome profile from a host, and providing a plurality of reference microbiome profiles, then selecting the reference profile most similar to the host microbiome profile, such that if the host's microbiome is most similar to a reference diseased microbiome, the host is at risk for that condition and/or disease. The microbiome profile from the host may be determined using an array of the invention. The reference profiles may be stored on a computer-readable medium such that software known in the art and detailed in the examples may be used to compare the microbiome profile and the reference profiles.

The host microbiome may be derived from a subject that is a human.

Kits

The present invention also encompasses a kit for evaluating a compound, therapeutic, or drug. Typically, the kit comprises an array and a computer-readable medium. The array may comprise a substrate, the substrate having disposed thereon at least one biomolecule that is modulated in a diseased host microbiome compared to a healthy host microbiome. The computer-readable medium may have a plurality of digitally-encoded profiles wherein each profile of the plurality has a plurality of values, each value representing the abundance of a biomolecule in a host microbiome detected by the array. The array may be used to determine a profile for a particular host under particular conditions, and then the computer-readable medium may be used to determine if the profile is similar to known profile stored on the computer-readable medium. Non-limiting examples of possible known profiles include profiles for individuals with Down syndrome and typical individuals.

### Epigename

The inheritance of information based on gene expression levels is known as epigenetics, as opposed to genetics, which refers to information transmitted on the basis of gene sequence. Cancer, which includes any malignant neoplastic disease, including but not limited to solid tumors and hematologic malignancies, as well as premalignant conditions, are epigenetic diseases characterized by the generation of aberrant patterns of DNA methylation and histone modifications with dramatic consequences in gene expression and architectural organization of genomic information (Esteller, 2008, Ballestar, 2008). Epigenetic events represent important mechanisms by which gene expression is selectively activated or inactivated leading to functional and biological alterations, which accumulate during aging and are important in tumorigenesis (Fraga, 2007a). In utero exposures can lead to life-course imprinting in the offspring and potentially modify disease susceptibility and risk (Sinclair, 2007). The epigenome is reproduced during mitosis and can be inherited across generations. The innate plasticity of the epigenome also enables it to be reprogrammed by social, chemical, biological and physical factors (Dolinoy, 2008). Emerging evidence indicates that various epigenetic alterations common to most types of cancer, such as global histone modifications and DNA hypomethylation, are also observed in other chronic diseases (Wilson, 2007). In many cases, epigenetic modifications are reversible, thus providing an opportunity to reverse the chronic disease process and understand the impact of lifestyle choices on chronic disease susceptibility and risk (Herranz, 2007).

The stability of the genome and correct gene expression is maintained to a great extent by a perfectly preestablished pattern of DNA methylation and histone modifications. In cancer and other chronic diseases this scenario breaks down due to a sudden loss of global methylation associated with histone modifications which lead to genomic instability, chromosomal rearrangements, activation of transposable elements and retroviruses, microsatellite instability and aberrant gene expression (Guerrero-Preston, 2007, Esteller, 2006a). In cancer an interesting gene-specific phenomenon following global DNA hypomethylation has been widely studied whereby the regulatory regions (CpG islands) of certain tumor suppressor genes (such as BRCA1, hMLHI, and VHL) become hypermethylated, inactivating the gene as a consequence, whilst the regulatory regions of proto-oncogenes become hypomethylated thus leading to transcriptional activation of the oncogene (Esteller, 2007a, Esteller , 2006b). Thus global DNA hypomethylation is usually seen together with gene-specific hyper and hypomethylation in cancer and other chronic diseases (Ehlrich,2006). The global methylcytosine content of a large collection of normal tissues and tumors has been studied to begin to understand this mechanism in cancer and other diseases (Hoffmann, 2005).

The human epigenome is dynamic, not only throughout the cell cycle and during mitotic divisions, but also in its response to environmental factors, which can be critical in development and during aging (Fraga, 2007b). Transient and fixed epigenetic modifications continually modulate the normal human epigenome throughout the life course in response to endogenous and exogenous stimuli. The epigenome serves as an interface between the dynamic environment and the inherited static genome, configured during development to shape the diversity of gene expression programs in the different cell types of the organism by a highly organized process. It is has been shown that exposure to physical, biological and chemical factors, as well as exposure to social behavior, such as maternal care, modifies the epigenome (Szyf, 2008). Therefore exposures to different environmental agents throughout the life course may lead to interindividual phenotypic diversity, as well as differential susceptibility to disease and behavioral pathologies.

DNA methylation, the most important epigenetic modification known, is a chemical modification of the DNA molecule itself, which is carried out by an enzyme called DNA methyltransferase. DNA methylation can directly switch off gene expression by preventing transcription factors binding to promoters. However, a more general effect is the attraction of methyl-binding domain (MBD) proteins, These are associated with further enzymes called histone deacetylases (HDACs), which function to chemically modify histones and change chromatin structure. Chromatin containing acetylated histones is open and accessible to transcription factors, and the genes are potentially active. Histone deacetylation causes the condensation of chromatin, making it inaccessible to transcription factors and the genes are therefore silenced (Eberharter,2002). The link between histone deacetylation and DNA methylation was the finding that MeCP2 physically interacts with the transcriptional co-repressor protein Sin3A, and in so doing recruits a histone deacetylase (HDAC) to chromatin that contains methylated DNA (Tycko, 2000, Studnicki, 2005).

A recently published study (Fraga, 2005 a) examined the global and locus-specific differences in DNA methylation and histone acetylation of a large cohort of monozygotic twins, They found that, although twins are epigenetically indistinguishable during the early years of life, older monozygous twins exhibited remarkable differences in their overall content and genomic distribution of 5-methylcytosine DNA and histone acetylation, affecting their gene-expression portrait,

In one embodiment, an epigenetic change in a subject with Down syndrome indicates that the subject has an increased risk of being afflicted with a condition or disease more prevalent in individuals with Down syndrome, In another embodiment, an epigenetic change in a typical subject indicates that the typical subject has an increased risk of being afflicted with a condition or disease more prevalent in typical individuals and rare in individuals with Down syndrome. In another embodiment, the present invention is used as an epigenomic screening andior detecting tool for early detection of a condition or disease more prevalent in individuals with Down syndrome. In another embodiment, the present invention is used as an epigenomic screening and/or detecting tool for early detection of a condition or disease more prevalent in typical individuals. In another embodiment, the present invention is used as an epigenomic screening and/or detecting tool of recurrence of a condition or disease prevalent in an individual with Down syndrome after treatment, or as a biomarker of therapeutic effectiveness. In another embodiment, the present invention provides means to decrease mortality rates, increase survival rates and decrease overall disease associated health care expenditures, by improving detection, including early detection, detection of recurrences, measuring therapeutic effectiveness and monitoring modifiable lifestyle and contextual effects related to the disease.

### Induced Pluripotent Stem Cells

Induced pluripotent stem cells (iPSCs) are a type of pluripotent stem cell that can be generated directly from adult cells. Pluripotent stem cells hold great promise in the field of regenerative medicine. Because they can propagate indefinitely, as well as give rise to every other cell type in the body (such as neurons, heart, pancreatic, and liver cells), they represent a single source of cells that could be used to replace those lost to damage or disease, iPSCs are currently being used in personalized drug discovery efforts and understanding the patient-specific basis of disease.

iPSCs are typically derived by introducing a specific set of pluripotency-associated genes, or “reprogramming factors”, into a given cell type. The original set of reprogramming factors (also dubbed Yamanaka factors) are the genes Oct4 (Pou5f1), Sox2, cMyc, and Klf4. While this combination is most conventional in producing iPSCs, each of the factors can be functionally replaced by related transcription factors, miRNAs, small molecules, or even non-related genes such as lineage specifiers.

iPSC derivation is typically a slow and inefficient process, taking 1-2 weeks for mouse cells and 3-4 weeks for human cells, with efficiencies around 0.01%-0.1%. However, considerable advances have been made in improving the efficiency and the time it takes to obtain iPSCs. Upon introduction of reprogramming factors, cells begin to form colonies that resemble pluripotent stem cells, which can be isolated based on their morphology, conditions that select for their growth, or through expression of surface markers or reporter genes.

Human iPSCs can be generated from human fibroblasts using a retroviral or lentiviral system to transform the cells with reprogramming factors.

An attractive feature of human iPS cells is the ability to derive them from adult patients to study the cellular basis of human disease. Since iPS cells are self-renewing and pluripotent, they represent a theoretically unlimited source of patient-derived cells which can be turned into any type of cell in the body. This is particularly important because many other types of human cells derived from patients tend to stop growing after a few passages in laboratory culture. iPS cells have been generated for a wide variety of human genetic diseases, including common disorders such as Down syndrome and polycystic kidney disease. In many instances, the patient-derived iPS cells exhibit cellular defects not observed in iPS cells from healthy patients, providing insight into the pathophysiology of the disease.

In another embodiment, the invention pertains to pharmaceutical compositions comprising a biologically active small molecule, disease relevant, or another molecule obtained through using the methods of the invention and a pharmaceutically acceptable carrier. In another embodiment, the invention includes nutraceutical preparations of biologically active small molecules of the invention.

The biologically active small molecules may be chemically modified to enhance their biological activity. It is known in the art that through chemical modifications, one can enhance the biological activity, stability, or otherwise modify a molecule to make it more suitable as a pharmaceutical or nutraceutical agent.

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body, Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations,

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about percent to about 70 percent, most preferably from about 10 percent to about 30 percent.

Methods of preparing these formulations or compositions include the step of bringing into association a compound of the present invention with the carrier and, optionally one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent,

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel,

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbin, acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, topically, or rectally, They are of course given by forms suitable for each administration route. For example, they are administered in tablets or capsule form, by injection, inhalation, eye lotion, ointment, suppository, etc, administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician having ordinary skill in the art can readily determine and prescribe the effective amount of the pharmaceutical composition required, For example, the physician could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

As set out above, certain embodiments of the present compounds can contain a basic functional group, such as amino or alkylamino, and are, thus, capable of forming pharmaceutically acceptable salts with pharmaceutically acceptable acids. The term “pharmaceutically acceptable salts” is art recognized and includes relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

In other cases, the compounds of the present invention may contain one or more acidic functional groups and, thus, are capable of forming pharmaceutically acceptable salts with pharmaceutically acceptable bases. The term “pharmaceutically acceptable salts” in these instances includes relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. Hydroxyls can be converted into esters via treatment with an esterifying agent such as alkanoyl halides. The term also includes lower hydrocarbon groups capable of being solvated under physiological conditions, e.g., alkyl esters, methyl, ethyl and propyl esters. (See, for example, Berge et al., supra.)

### EXAMPLE 1

### Proteomics Studies of the Systemic Signaling Changes Caused by Trisomy 21

Proteomics studies were undertaken to: 1) identify systemic signaling events caused by trisomy 21, 2) identify biomarkers of the ‘natural history’ of Down syndrome (i.e. age-dependent molecular events), and 3) identify biomarkers of specific co-morbidities associated with Down syndrome.

Discovery Study

Blood samples were collected from 121 individuals with Down syndrome and 54 control subjects. The blood samples were separated into plasma, white blood cells and red blood cells.

Binding. Modified aptamers (Gold et al, (2010), PLoS ONE 5(12): e15004; Rohloff et al. (2014), Molecular Therapy—Nucleic Acids (2014) 3, e201) and samples were mixed in 96-well microwell plates and allowed to bind. Cognate and non-cognate aptamer-target protein complexes were allowed to form. Free aptamers and protein were also present,

DNA-based aptamer molecules have unique shapes selected to bind to a specific protein. Aptamers contain biotin, a photo-cleavable linker and a fluorescent tag at the 5′ end. Most aptamers bind to cognate proteins, but some aptamers form non-cognate complexes.

Catch-1. Aptamers were captured onto a bead coated with streptavidin which binds biotin. Un-complexed proteins were washed away, Proteins were then tagged with NHS-biotin. Subsequently, UV light (hv) was used to cleave the linker and aptamers were released from beads, leaving biotin on the beads. Samples were challenged with anionic competitor (dextran sulfate) to preferentially dissociate non-cognate complexes.

Catch-2. Aptamer-protein complexes were captured onto new avidin coated beads by the protein biotin tag. Free aptamers were washed away, Aptamers were released from complexes into solution at high pH and quantified by hybridization to a microarray containing single-stranded DNA probes complementary to aptamer DNA sequence. Hybridized aptamers were detected by fluorescent tags when the array was scanned.

The capture of aptamers on a hybridization array permits quantitative determination of the protein present in the original sample by converting the assay signal (relative fluorescence units, RFUs) to analyte concentration. Thus, the assay takes advantage of the dual nature of aptamers as molecules capable of both folding into complex three-dimensional structures, which is the basis of their unique binding properties, and hybridization to specific capture probes.

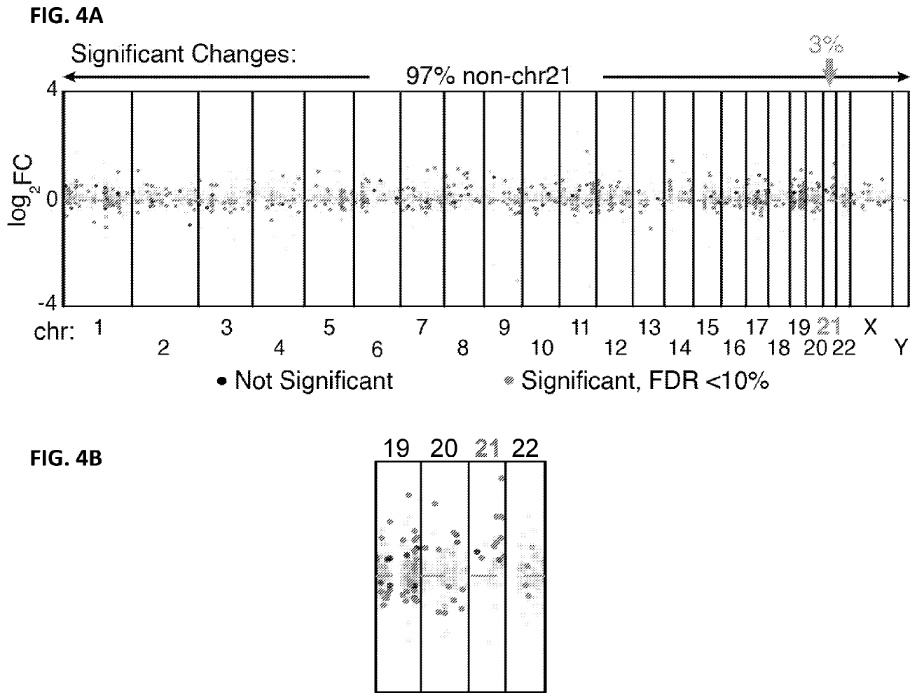

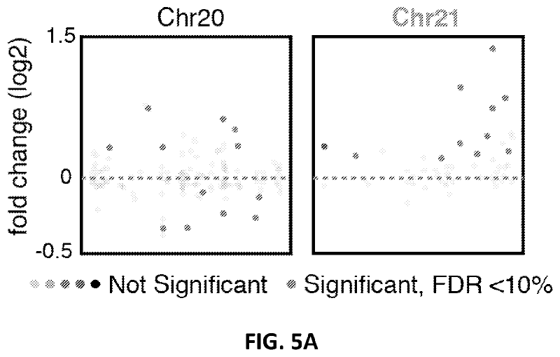

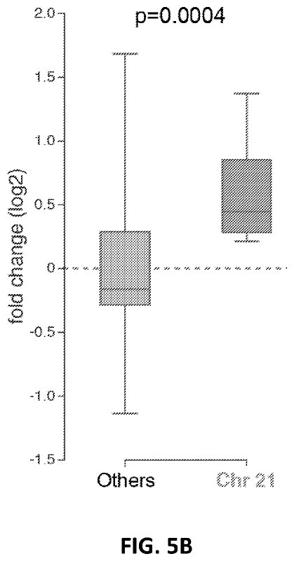

A total of 3624 proteins were identified in the study. 97% of significant protein expression changes were on chromosomes other than chromosome 21, while 3% of significant protein expression changes were on chromosome 21 (FIG. 4A). Proteins on chromosome 21 tended to be upregulated, with some reaching statistical significance, but none were significantly downregulated (FIG. 4B), FIGS. 5A and 5B show that significant protein expression changes on chromosome 21 were all increases.

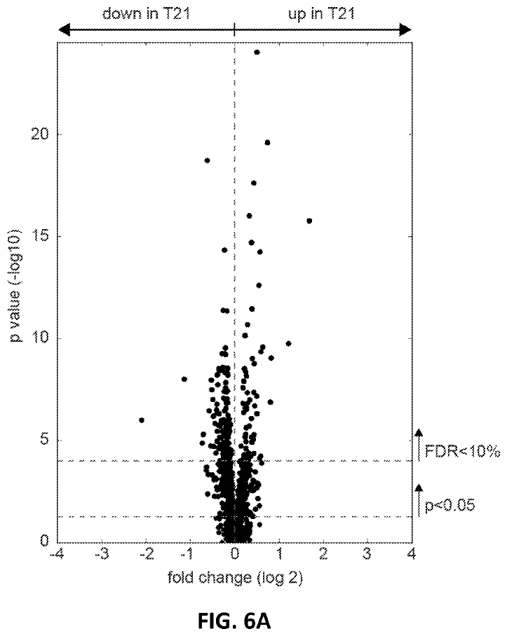

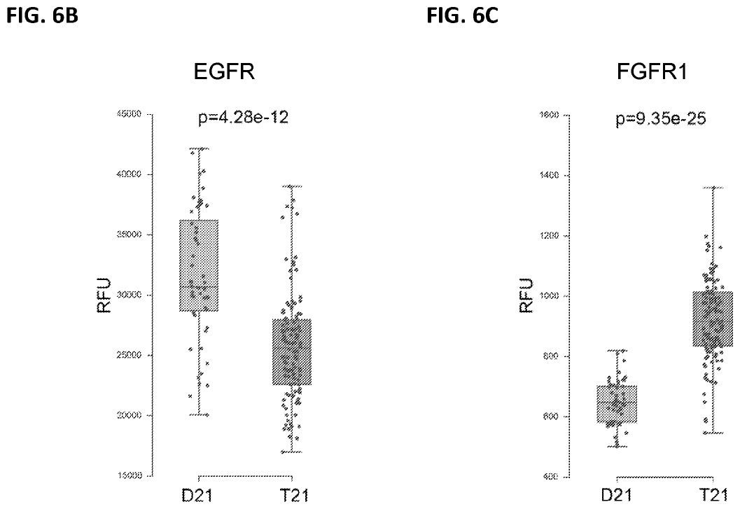

Trisomy 21 causes significant changes in the systemic proteome. FIG. 6A shows that expression changes in a proportion of 1129 proteins from the Discovery Study had p values <0.05 and many had a false discovery rate (FDR) of <10%, For example, epidermal growth factor receptor (EGFR) expression was significantly downregulated in individuals with Down syndrome compared to typical individuals (FIG. 6B), while fibroblast growth factor receptor 1 (FGFR1) expression was significantly upregulated in individuals with Down syndrome compared to typical individuals (FIG. 6C).

Proteins identified in the above-described method showing increased expression in individuals with Down syndrome relative to typical individuals (q<0.05) are shown in Table 1.

Proteins identified in the above-described method showing decreased expression in individuals with Down syndrome relative to typical individuals (q <0.05) are shown in Table 2.

### Validation Study 1

Blood samples were collected from 57 participants, 41 with Down syndrome. Thirty-eight of the participants were repeats from the Discovery Study. Proteomics analysis was performed as described for the Discovery Study. A total of 1129 proteins were identified in the study.

### Validation Study 2

Blood samples were collected from 42 participants, 22 with Down syndrome. All participants were new. Proteomics analysis was performed as described for the Discovery Study. A total of 1129 proteins were identified in the study.

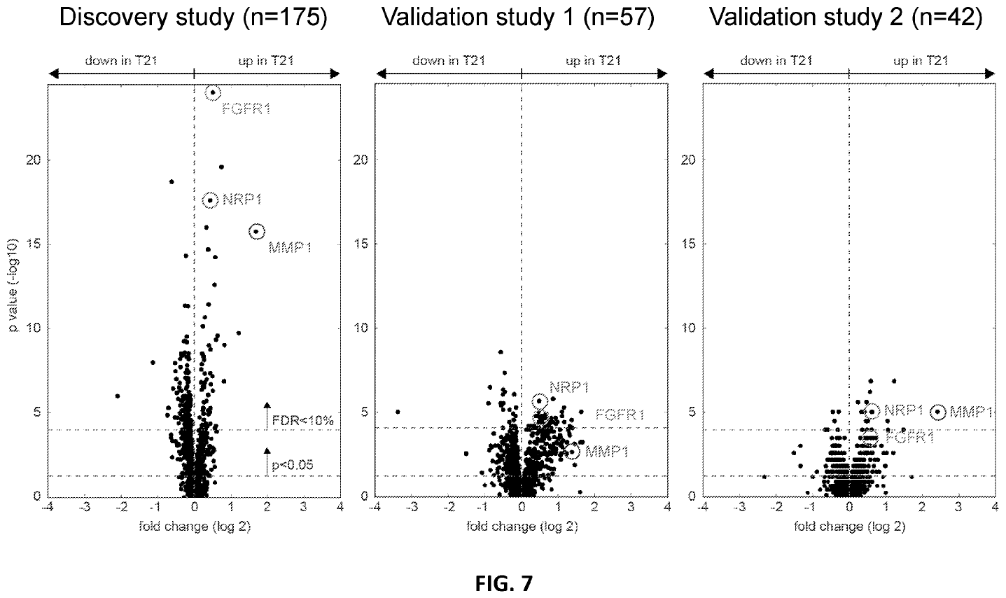

Significant changes in the systemic proteome caused by trisomy 21 were also seen in the two validation studies (FIG. 7). For example, the Discovery study found that FGFR1, neuropilin (NRP1) and matrix metalloproteinase 1 (MMPI) expression were all significantly upregulated in individuals with Down syndrome compared to typical individuals, with a FDR<10%. Validation study 1 found that both NRP1 and FGFR1 were significantly upregulated in individuals with Down syndrome compared to typical individuals with a FDR <10%, and MMP1 was significantly upregulated at p<0.05. Validation study 2 found that both NRP1 and MMPI were significantly upregulated with a FDR<10%, and FGFR1 was significantly upregulated at p<0.05.

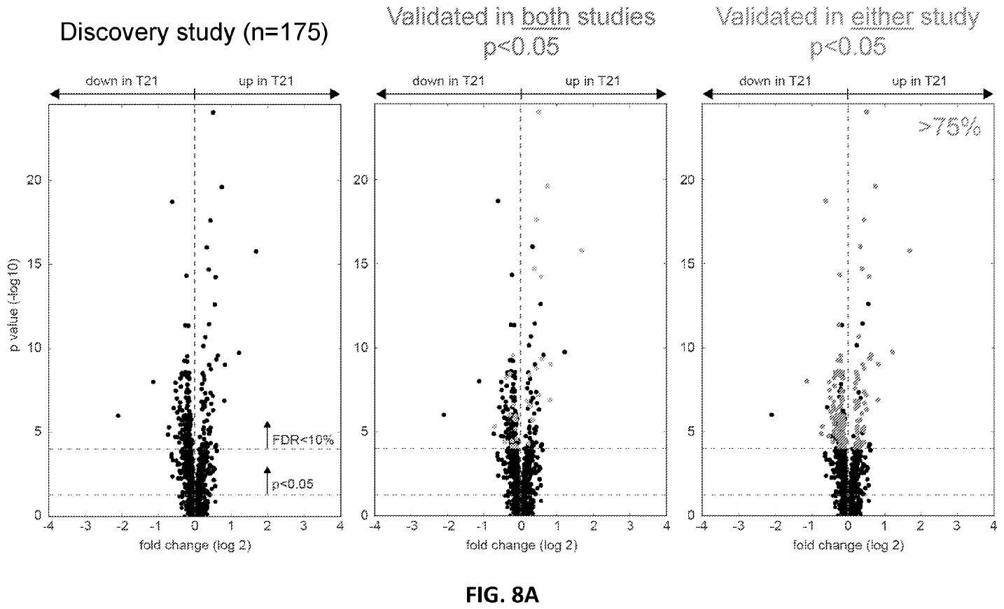

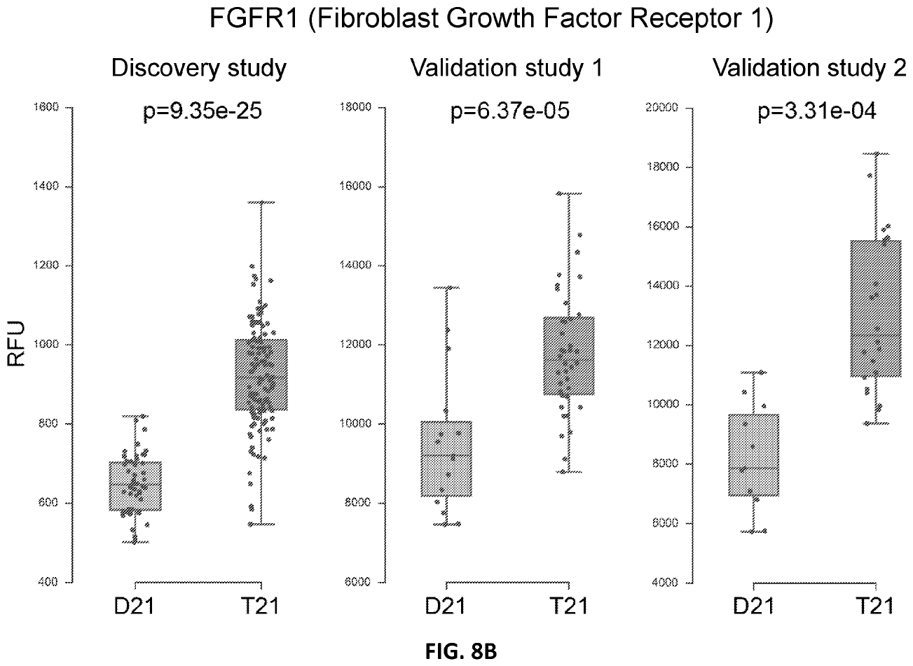

The two validation studies also demonstrated that changes in the systemic proteome are reproducible. FIG. 8A shows the proportion of upregulated and downregulated proteins identified in the Discovery study that were validated by both validation studies (FIG. 8A, middle panel) at p<0.05, or by one of the validation studies (FIG. 8A, right panel) at p<0.05. FIG. 8B shows that FGFR1 expression was significantly upregulated in individuals with Down syndrome compared to typical individuals in all three studies (Discovery, Validation 1 and Validation 2).

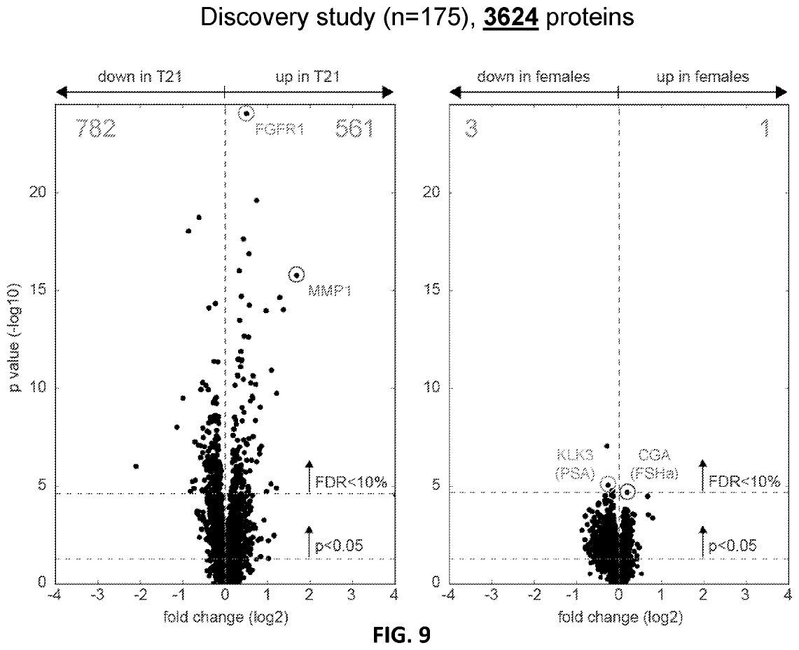

Trisomy 21 causes massive changes in the systemic proteome. FIG. 9 (left panel) shows that the expression of 782 proteins was significantly downregulated at a 5% FDR, while the expression of 561 proteins was significantly upregulated at a 5% FDR, in individuals with Down syndrome compared to typical individuals in the Discovery study. These changes in protein expression are much more drastic than those caused by gender, as demonstrated in FIG. 9 (right panel), where only the expression of 3 proteins was significantly downregulated at a 5% FDR and the expression of 1 protein was significantly upregulated at a 5% FDR, in typical females compared to typical males.

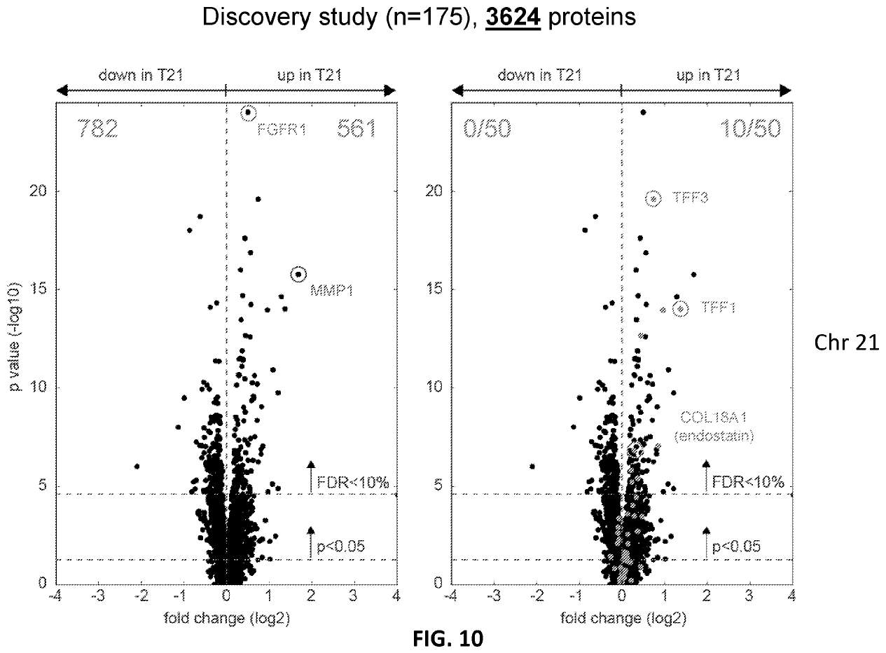

Most protein expression changes caused by trisomy 21 occur in the proteome associated with chromosomes other than chromosome 21. Of the 3624 proteins downregulated or upregulated in individuals with Down syndrome compared to typical individuals in the Discovery study, fifty are encoded by genes on chromosome 21 (FIG. 10, right panel). None of the 50 proteins are downregulated, whereas 10 of the 50 are upregulated. Examples of significantly upregulated chromosome 21 proteins include Trefoil factor 3 (TFF3), Trefoil factor 1 (TFF1) and COL18A1 (endostatin) (FIG. 10, right panel, circled dots).

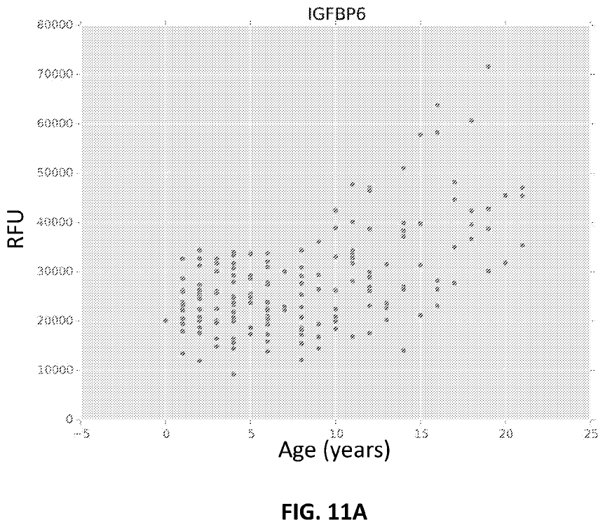

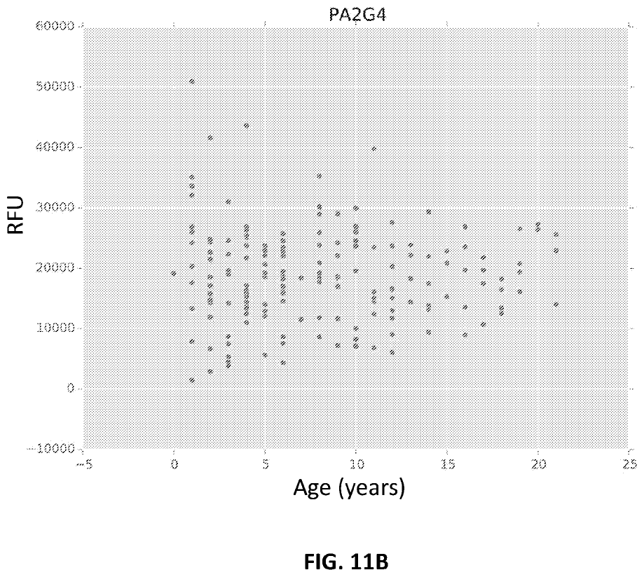

Levels of many proteins change with age. For example, insulin-like growth factor binding protein 6 (IGBP6) is involved in insulin-like growth factor (IGF) transport in blood. IGBP6 is anti-proliferative and pro-apoptotic. Proteomics studies across all ages indicate that IGBP6 increases with age from age 9 in both individuals with Down syndrome and in typical individuals (FIG. 11A). By contrast, the level of ERBB3 binding protein, a proliferation-associated co-repressor of androgen-regulated genes that interacts with histone deacetylases, increases with age in typical individuals but decreases in individuals with Down syndrome (FIG. 11B),

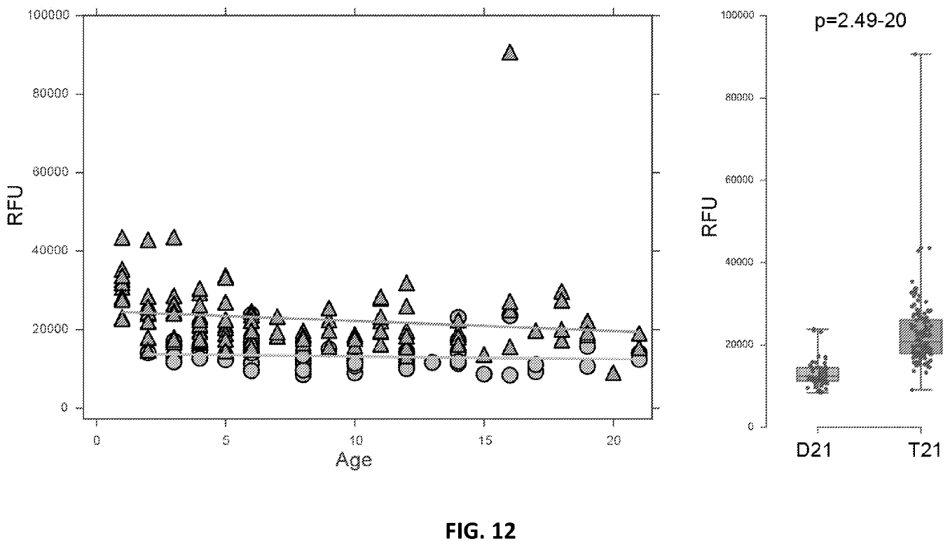

TFF3, encoded by a gene on chromosome 21, is a stable secretory protein from intestinal mucosa related to IL4 signaling and hypoxia-inducible factor 1-alpha (HIF1alpha), a master transcriptional regulator of cellular and developmental response to hypoxia. FIG. 12 shows that TFF3 protein expression is upregulated in individuals with Down syndrome compared to typical individuals (right panel), and TFF3 protein decreases with increasing age in individuals with Down Syndrome, while it increases with increasing age in typical individuals.

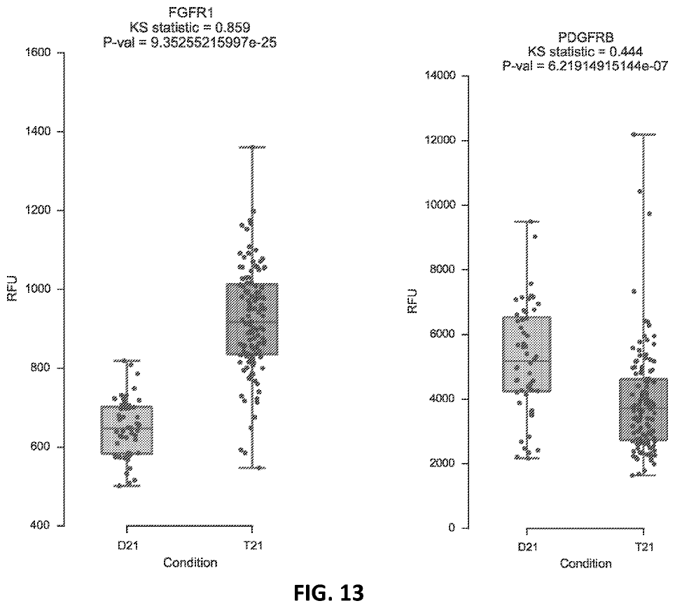

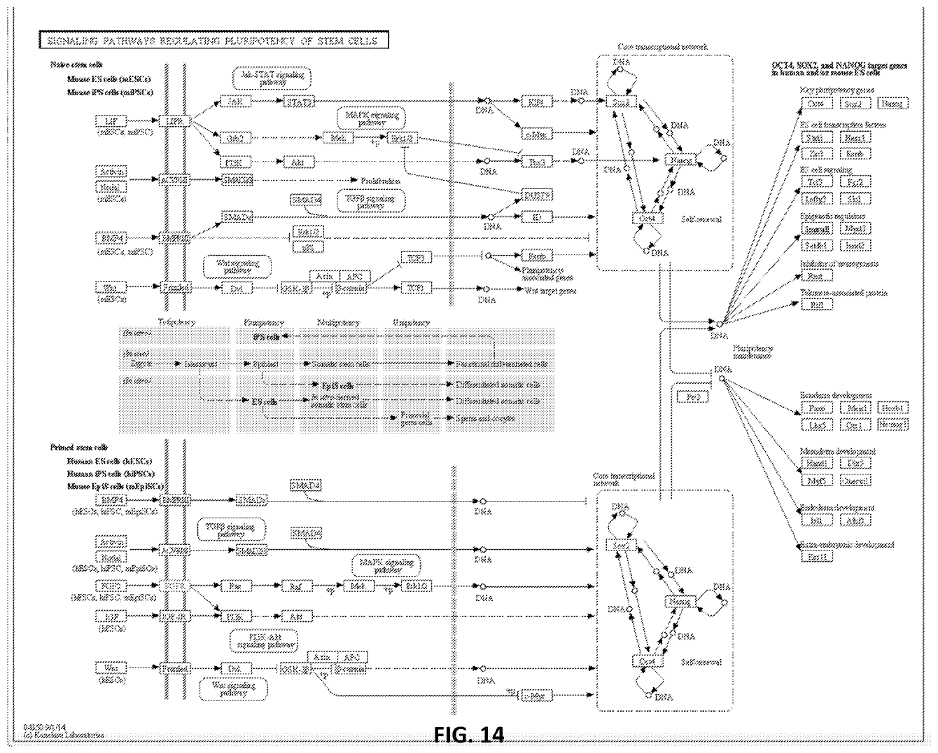

As mentioned above, there is a large consistent upregulation in expression of FGFR1, while the expression of platelet-derived growth factor receptor (PDGFR) is downregulated in individuals with Down syndrome compared to typical individuals (FIG. 13). Overexpression of FGFR in Pfeiffer, Jackson-Weiss and Antley-Bixler syndromes causes skeletal abnormalities and wide-set eyes. FGFR plays a role in regulating pluripotency of stem cells (FIG. 14).

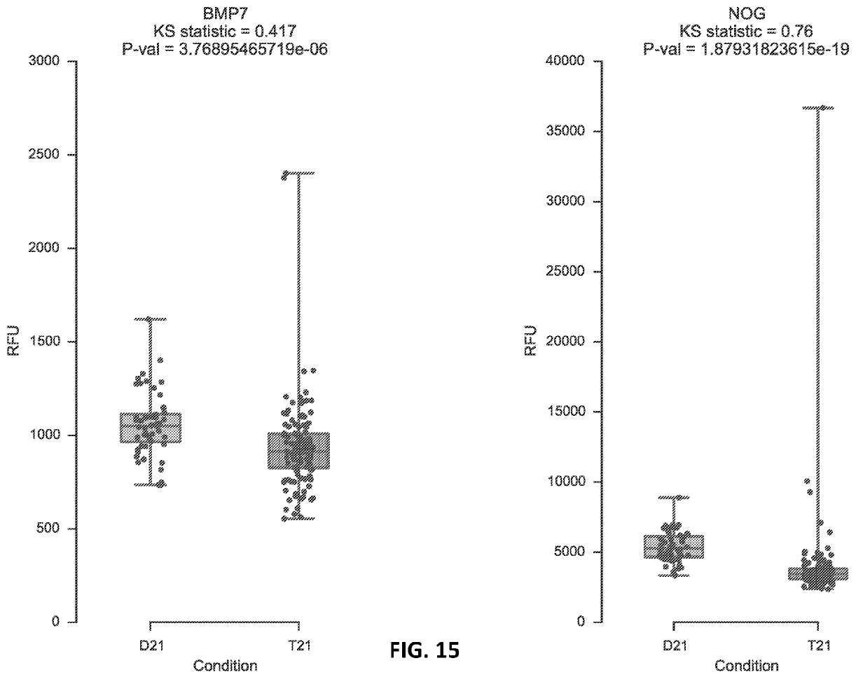

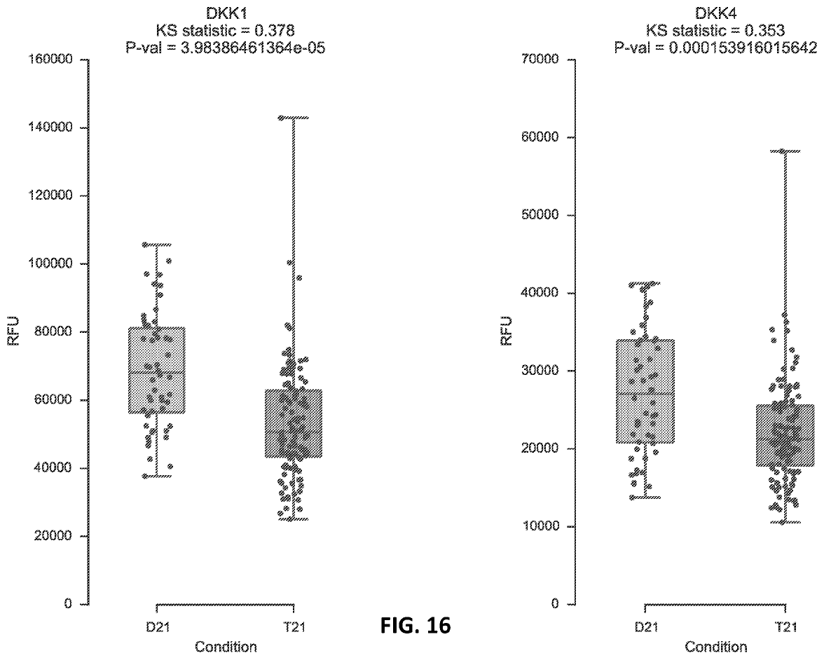

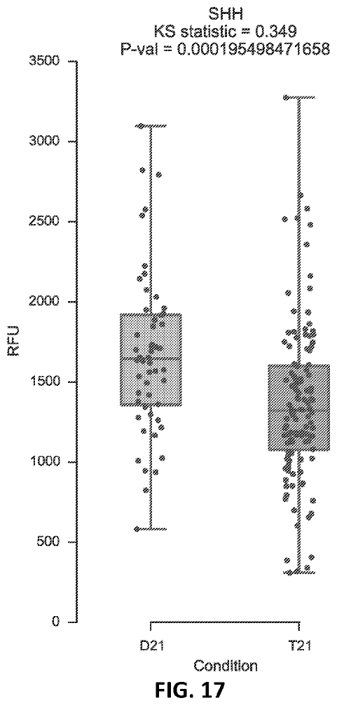

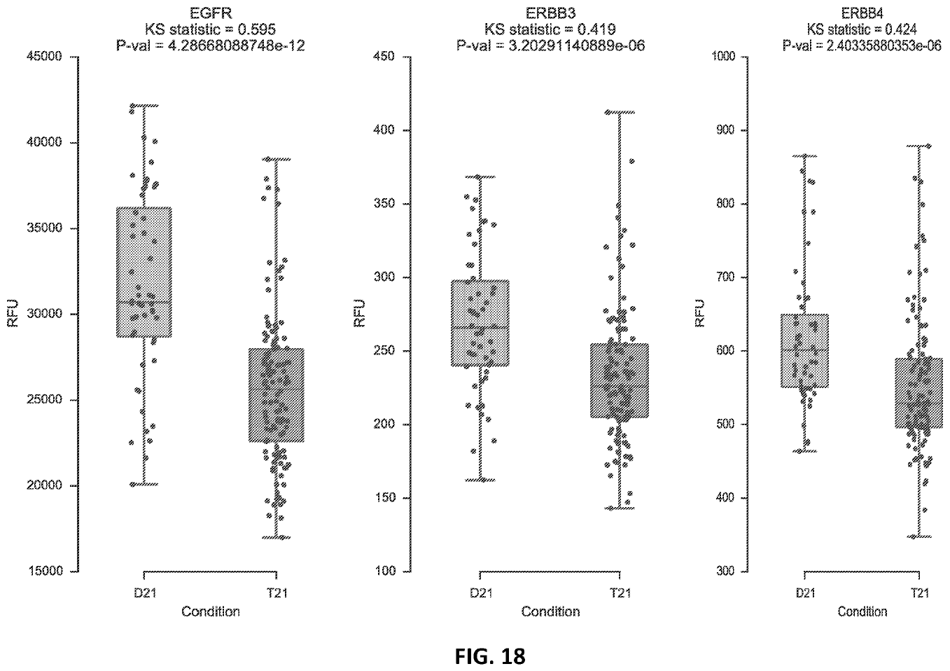

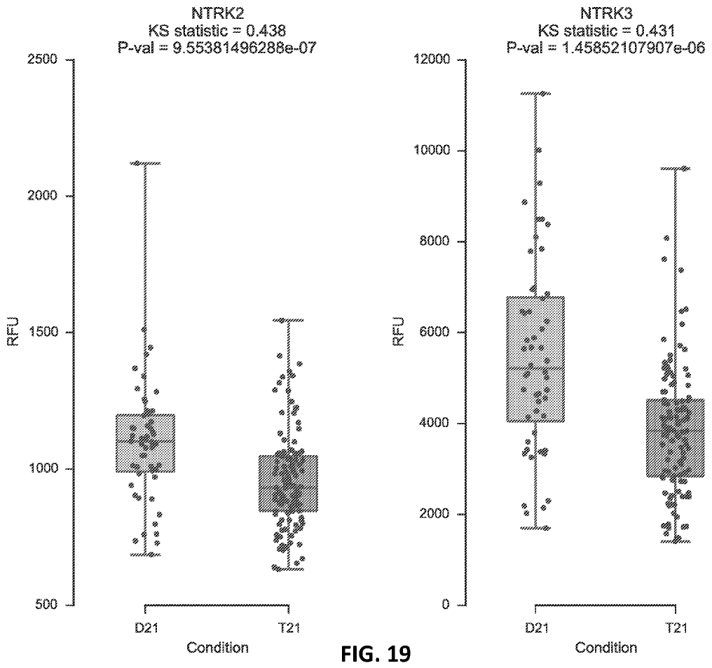

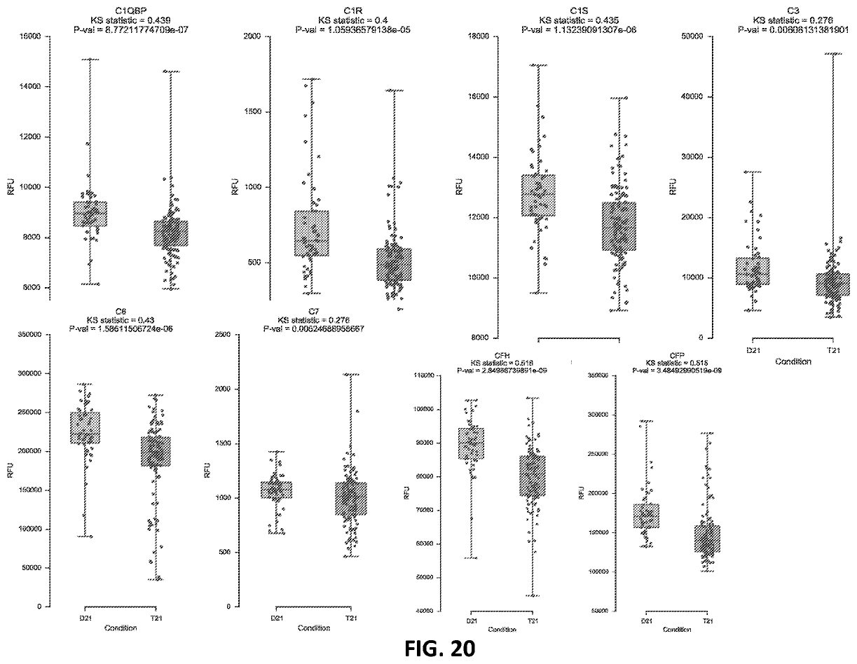

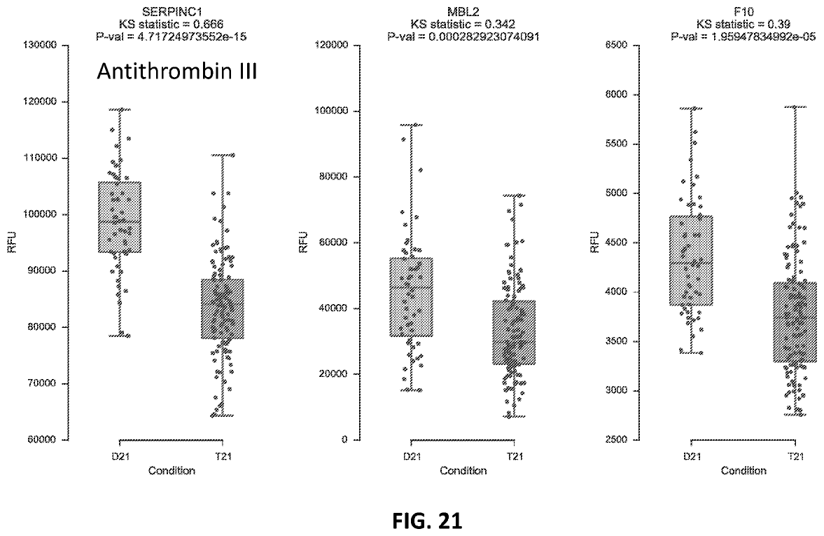

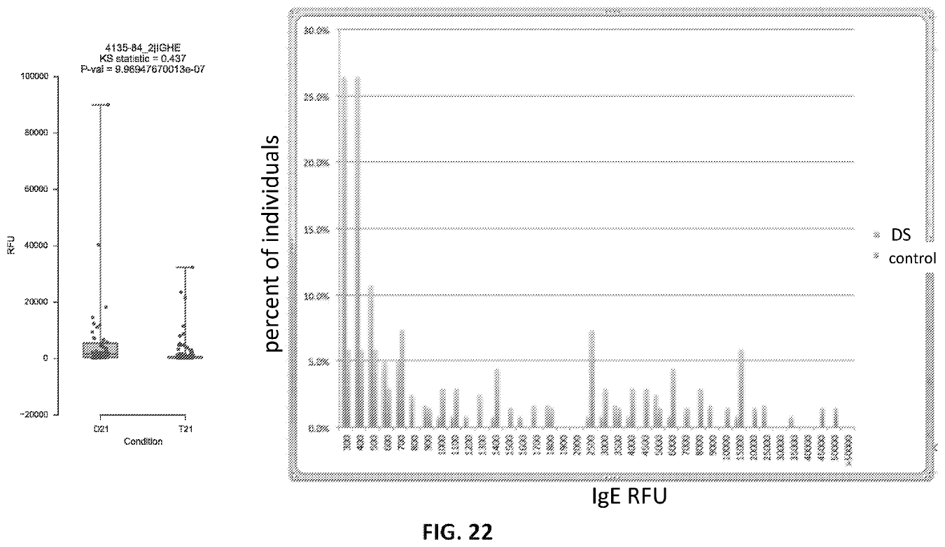

A number of proteins are downregulated in individuals with Down syndrome compared to typical individuals (see Table 2), These include signaling peptides involved in bone and limb development such as BMP7 and NOG (FIG. 15), Wnt inhibitors such as DKK1 and DKK4 (FIG. 16), Sonic Hedgehog (SHH) (FIG. 17), epidermal growth factor receptors (EFGR, ERBB3, ERBB4) that are activated by neuregulins-2 and -3 (FIG. 18), neurotrophin receptors such as TrkB and TrkC (FIG. 19), proteins in the complement cascade (FIG. 20), proteins in the coagulation cascade (FIG. 21) and IgE (FIG. 22). For example, factor P (OFF) is a positive regulator of complement activation and is downregulated in individuals with Down syndrome compared to typical individuals (FIG. 20). In the coagulation cascade, low levels of Cl inhibitor and/or factor Xa can result in thrombosis and pulmonary embolism (FIG. 21).

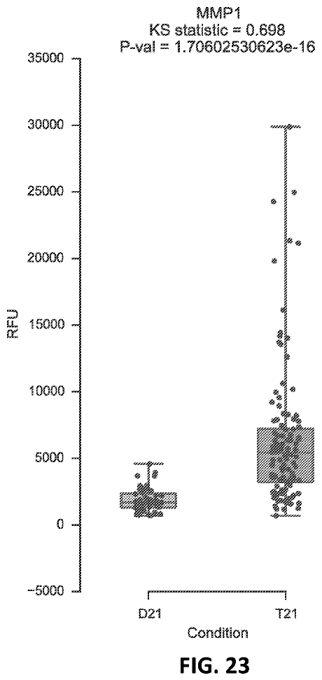

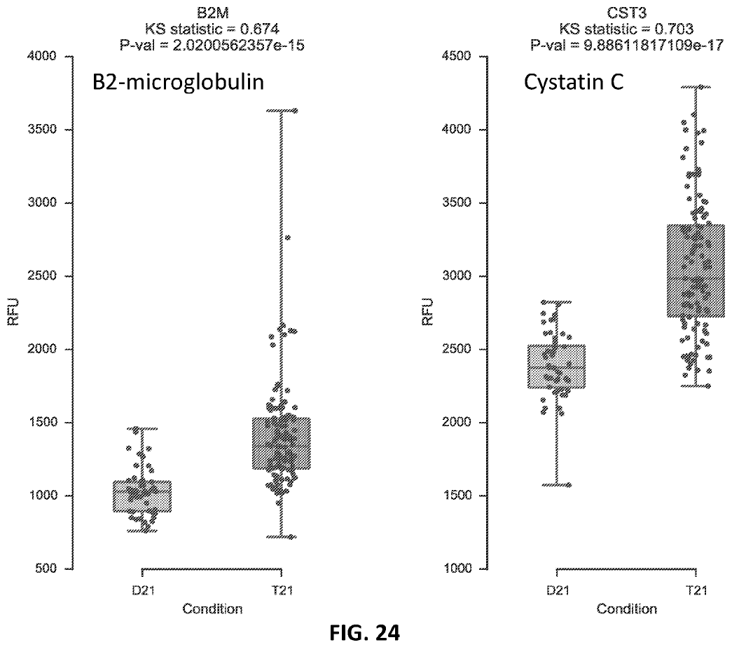

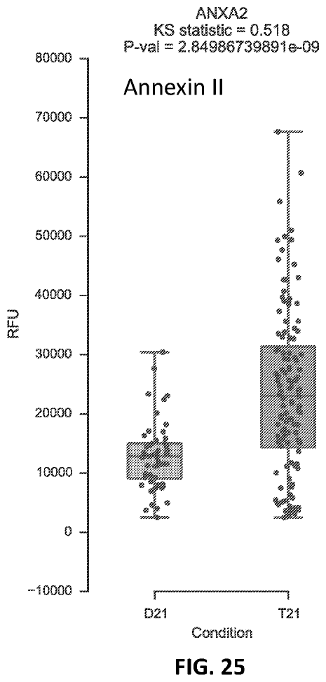

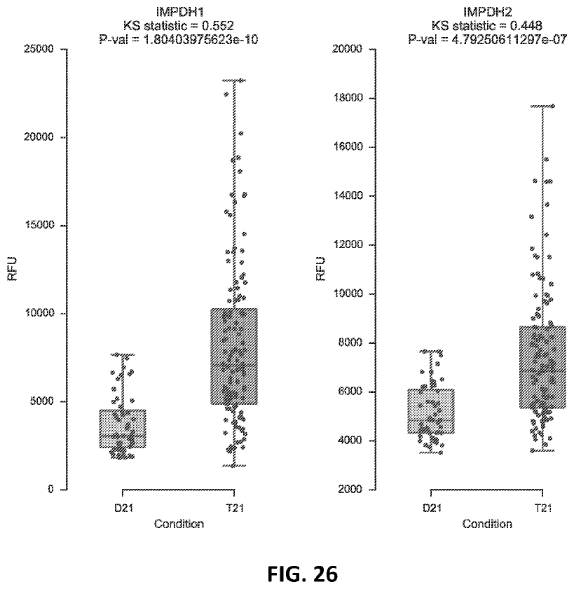

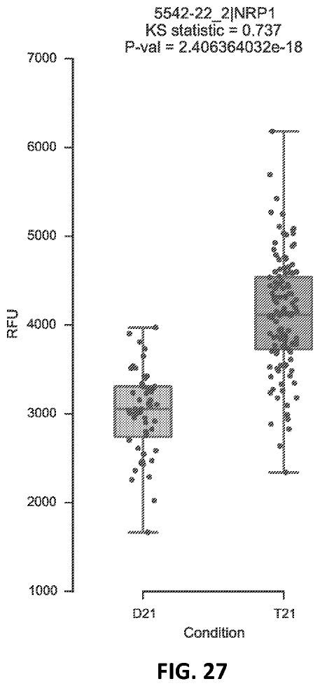

A number of proteins are upregulated in individuals with Down syndrome compared to typical individuals. These include MMPI (FIG. 23), a matrix metalloproteinase needed for breakdown of extracellular matrix, which is increased>300% in individuals with Down syndrome; proteins associated with impaired kidney function such as B2-microglobulin and Cystatin C (FIG. 24); Annexin II, an autocrine factor that heightens osteoclast formation and bone resorption and regulates cell growth and signal transduction, in part by binding to factor H, among others (FIG. 25); inosine 5′ monophosphate dehydrogenases 1 and 2 (IMPDH1 and IMPDH2), which acts in the rate limiting step for GTP synthesis, is a regulator of cell growth and is mutated in Retinitis pigmentosa (FIG. 26); and neuropilin (NRPI), a receptor for vascular endothelial growth factor (VEGF) and semaphorin and which has roles in axon guidance and angiogenesis (FIG. 27).

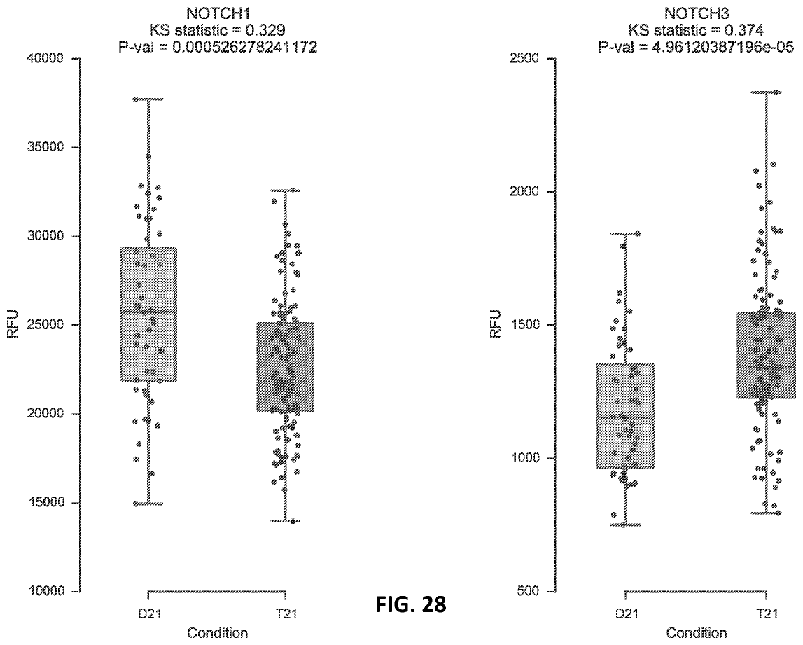

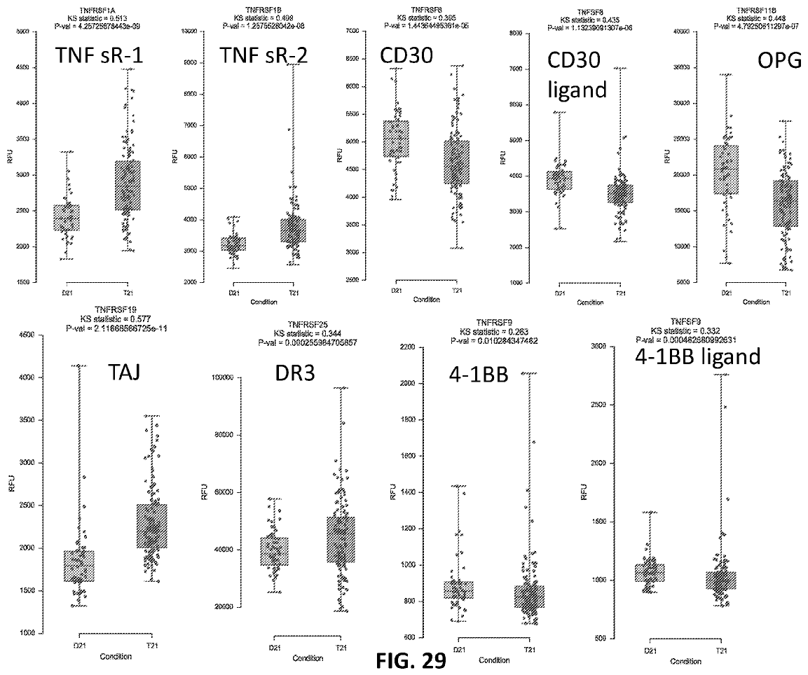

Notch proteins exhibit both upregulation and downregulation in individuals with Down syndrome compared to typical individuals (FIG. 28). Similarly, receptors and ligands associated with tumor necrosis factor are both upregulated and downregulated (FIG. 29).

Future experiments will involve; assessing which genes on chromosome 21, when overexpressed, cause which protein expression changes; determining the consequences of protein expression changes for individuals with Down syndrome; determining which protein expression changes correlate with which phenotypes; assessing which protein expression changes have functional consequences; determining whether some protein expression changes are attempts at compensations and if so, which changes are functionally important.

Though not wishing to be bound by any theory, one hypothesis to explain the molecular basis of trisomy 21 is that the extra chromosome causes global gene expression changes and production of extra proteins, either directly or indirectly. The stress response is induced, and so cell division is suppressed. This causes premature differentiation of dividing cells during development and later. Many stem cells senesce or die. Many are made at low levels. Levels of hematopoietic cells are altered, resulting in under proliferation of some cell types and over proliferation of others. Some protein level changes are a consequence of this alteration in the abundance of hematopoietic cell types. One of skill in the art can posit other hypotheses.

TABLE 1
Exemplary biomarkers with increased expression from proteome analysis
of individuals with Down syndrome compared to typical individuals.

| | Chr | | | | Entrez Gene | EntrezGene | fold |
|---|---|---|---|---|---|---|---|
| | 21 | Target Full Name | Target | UniProt | ID | Symbol | change |
| 1 | N | Fibroblast growth | bFGF-R | P11362 | 2260 | FGFR1 | 1.37442 |
| | | factor receptor 1 | | | | | |
| 2 | Y | Trefoil factor 3 | TFF3 | Q07654 | 7033 | TFF3 | 1.670249 |
| 3 | N | Cystatin-C | Cystatin C | P01034 | 1471 | CST3 | 1.293731 |
| 4 | N | Inosine-5′- | IMDH1 | P20839 | 3614 | IMPDH1 | 2.406145 |
| | | monophosphate | | | | | |
| | | dehydrogenase 1 | | | | | |
| 5 | N | Beta-2-microglobulin | b2- | P61769 | 567 | B2M | 1.295589 |
| | | | Microglobulin | | | | |
| 6 | N | Interstitial | MMP-1 | P03956 | 4312 | MMP1 | 3.003556 |
| | | collagenase | | | | | |
| 7 | N | Diablo homolog, | SMAC | Q9NR28 | 56616 | DIABLO | 1.201496 |
| | | mitochondrial | | | | | |
| 8 | N | Insulin-like growth | IGFBP-6 | P24592 | 3489 | IGFBP6 | 1.502164 |
| | | factor-binding | | | | | |
| | | protein 6 | | | | | |
| 9 | N | Annexin A2 | annexin II | P07355 | 302 | ANXA2 | 1.905921 |
| 10 | N | Inosine-5′- | IMDH2 | P12268 | 3615 | IMPDH2 | 1.52463 |
| | | monophosphate | | | | | |
| | | dehydrogenase 2 | | | | | |
| 11 | N | Cathepsin H | Cathepsin | P09668 | 1512 | CTSH | 1.14205 |
| | | | H | | | | |
| 12 | N | Delta-like protein 1 | DLL1 | O00548 | 28514 | DLL1 | 1.21593 |
| 13 | N | Endoplasmic | ERP29 | P30040 | 10961 | ERP29 | 1.193188 |
| | | reticulum resident | | | | | |
| | | protein 29 | | | | | |
| 14 | N | Tumor necrosis | TNF sR-I | P19438 | 7132 | TNFRSF1A | 1.179041 |
| | | factor receptor | | | | | |
| | | superfamily member | | | | | |
| | | 1A | | | | | |
| 15 | N | Alcohol | AK1A1 | P14550 | 10327 | AKR1A1 | 1.532019 |
| | | dehydrogenase | | | | | |
| | | [NADP(+)] | | | | | |
| 16 | N | Resistin | resistin | Q9HD89 | 56729 | RETN | 1.417031 |
| 17 | N | Tumor necrosis | TNF sR-II | P20333 | 7133 | TNFRSF1B | 1.129573 |
| | | factor receptor | | | | | |
| | | superfamily member | | | | | |
| | | 1B | | | | | |
| 18 | N | Elafin | Elafin | P19957 | 5266 | PI3 | 1.491752 |
| 19 | N | Tumor necrosis | TAJ | Q9NS68 | 55504 | TNFRSF19 | 1.167072 |
| | | factor receptor | | | | | |
| | | superfamily member | | | | | |
| | | 19 | | | | | |
| 20 | N | Integrin alpha-I: | Integrin | P56199, | 3672 3688 | ITGA1 ITGB1 | 2.186051 |
| | | beta-1 complex | a1b1 | P05556 | | | |
| 21 | N | Tumor necrosis | DR3 | Q93038 | 8718 | TNFRSF25 | 1.231431 |
| | | factor receptor | | | | | |
| | | superfamily member | | | | | |
| | | 25 | | | | | |
| 22 | Y | Endostatin | Endostatin | P39060 | 80781 | COL18A1 | 1.214101 |
| 23 | N | Heat shock cognate | HSP70 | P11142 | 3312 | HSPA8 | 1.152325 |
| | | 71 kDa protein | protein 8 | | | | |
| 24 | N | Tyrosine-protein | ROR1 | Q01973 | 4919 | ROR1 | 1.386535 |
| | | kinase | | | | | |
| | | transmembrane | | | | | |
| | | receptor ROR1 | | | | | |
| 25 | N | C-C motif chemokine | TARC | Q92583 | 6361 | CCL17 | 1.582712 |
| | | 17 | | | | | |
| 26 | N | Protein SET | SET | Q01105 | 6418 | SET | 1.115326 |
| 27 | N | CMRF35-like | CLM6 | Q08708 | 10871 | CD300C | 1.239697 |
| | | molecule 6 | | | | | |
| 28 | N | Insulin-degrading | IDE | P14735 | 3416 | IDE | 1.222896 |
| | | enzyme | | | | | |
| 29 | N | Vascular endothelial | VEGF121 | P15692 | 7422 | VEGFA | 1.366822 |
| | | growth factor A, | | | | | |
| | | isoform 121 | | | | | |
| 30 | N | Inorganic | PPase | Q15181 | 5464 | PPA1 | 1.302382 |
| | | pyrophosphatase | | | | | |
| 31 | N | Myeloid cell surface | Siglec-3 | P20138 | 945 | CD33 | 1.397446 |
| | | antigen CD33 | | | | | |
| 32 | N | Antileukoproteinase | SLPI | P03973 | 6590 | SLPI | 1.194221 |
| 33 | N | 40S ribosomal | RS7 | P62081 | 6201 | RPS7 | 1.233197 |
| | | protein S7 | | | | | |
| 34 | N | Follistatin-related | FSTL3 | O95633 | 10272 | FSTL3 | 1.232093 |
| | | protein 3 | | | | | |
| 35 | N | Nidogen-1 | Nidogen | P14543 | 4811 | NIDI | 1.134464 |
| 36 | N | 14-3-3 protein sigma | STRATIFIN | P31947 | 2810 | SFN | 1.120443 |
| 37 | N | Transforming growth | TGF-b R III | Q03167 | 7049 | TGFBR3 | 1.174308 |
| | | factor beta receptor | | | | | |
| | | type 3 | | | | | |
| 38 | N | Neutrophil | Lipocalin 2 | P80188 | 3934 | LCN2 | 1.309723 |
| | | gelatinase-associated | | | | | |
| | | lipocalin | | | | | |
| 39 | N | Heterogeneous | hnRNP | P22626 | 3181 | HNRNPA2B1 | 1.195253 |
| | | nuclear | A2/B1 | | | | |
| | | ribonucleoproteins | | | | | |
| | | A2/B1 | | | | | |
| 40 | N | 40S ribosomal | RS3 | P23396 | 6188 | RPS3 | 1.281126 |
| | | protein S3 | | | | | |
| 41 | N | Fibroblast growth | bFGF | P09038 | 2247 | FGF2 | 1.707685 |
| | | factor 2 | | | | | |
| 42 | N | Macrophage-capping | CAPG | P40121 | 822 | CAPG | 1.161738 |
| | | protein | | | | | |
| 43 | N | Metalloproteinase | TIMP-1 | P01033 | 7076 | TIMP1 | 1.146823 |
| | | inhibitor 1 | | | | | |
| 44 | N | C-C motif chemokine | CCL28 | Q9NRJ3 | 56477 | CCL28 | 1.325929 |
| | | 28 | | | | | |
| 45 | N | Dynactin subunit 2 | Dynactin | Q13561 | 10540 | DCTN2 | 1.06138 |
| | | | subunit 2 | | | | |
| 46 | N | Carbonic anhydrase 1 | Carbonic | P00915 | 759 | CA1 | 1.502326 |
| | | | anhydrase | | | | |
| | | | I | | | | |
| 47 | N | Cathepsin B | Cathepsin | P07858 | 1508 | CTSB | 1.126635 |
| | | | B | | | | |
| 48 | N | Immunoglobulin G | IgG | P01857 | 3500 3501 | IGHG1 | 1.150859 |
| | | | | | 3502 3503 | IGHG2 | |
| | | | | | 50802 3535 | IGHG3 | |
| | | | | | | IGHG4 | |
| | | | | | | IGK@ IGL@ | |
| 49 | N | Toll-like receptor 2 | TLR2 | O60603 | 7097 | TLR2 | 1.027311 |
| 50 | N | Vascular endothelial | VEGF sR3 | P35916 | 2324 | FLT4 | 1.193395 |
| | | growth factor | | | | | |
| | | receptor 3 | | | | | |
| 51 | N | C-type lectin domain | CLC4K | Q9UJ71 | 50489 | CD207 | 1.016764 |
| | | family 4 member K | | | | | |
| 52 | N | Retinol-binding | RBP | P02753 | 5950 | RBP4 | 1.103923 |
| | | protein 4 | | | | | |
| 53 | N | Phospholipase A2, | NPS-PLA2 | P14555 | 5320 | PLA2G2A | 1.43174 |
| | | membrane | | | | | |
| | | associated | | | | | |
| 54 | Y | ICOS ligand | B7-H2 | O75144 | 23308 | ICOSLG | 1.225662 |
| 55 | N | Annexin A1 | annexin I | P04083 | 301 | ANXA1 | 1.203097 |
| 56 | N | Cyclin-dependent | CDK2/cyclin A | P24941 | 1017 890 | CDK2 | 1.080852 |
| | | kinase 2: Cyclin-A2 | | P20248 | | CCNA2 | |
| | | complex | | | | | |
| 57 | N | Gelsolin | Gelsolin | P06396 | 2934 | GSN | 1.14083 |
| 58 | N | Metalloproteinase | TIMP-2 | P16035 | 7077 | TIMP2 | 1.182796 |
| | | inhibitor 2 | | | | | |
| 59 | N | E-selectin | sE- | P16581 | 6401 | SELE | 1.195595 |
| | | | Selectin | | | | |
| 60 | N | Persulfide | ETHE1 | O95571 | 23474 | ETHE1 | 1.136471 |
| | | dioxygenase ETHE1, | | | | | |
| | | mitochondrial | | | | | |
| 61 | N | Eukaryotic | eIF-5 | P55010 | 1983 | EIF5 | 1.191584 |
| | | translation initiation | | | | | |
| | | factor 5 | | | | | |
| 62 | N | NKG2-D type II | NKG2D | P26718 | 22914 | KLRK1 | 1.181162 |
| | | integral membrane | | | | | |
| | | protein | | | | | |
| 63 | N | Hepatocyte growth | Met | P08581 | 4233 | MET | 1.100906 |
| | | factor receptor | | | | | |
| 64 | N | Neuroblastoma | DAN | P41271 | 4681 | NBL1 | 1.115438 |
| | | suppressor of | | | | | |
| | | tumorigenicity 1 | | | | | |
| 65 | N | Oxidized low-density | OLR1 | P78380 | 4973 | OLR1 | 1.54935 |
| | | lipoprotein receptor | | | | | |
| | | 1 | | | | | |
| 66 | N | Killer cell | KI3L2 | P43630 | 3812 | KIR3DL2 | 1.419006 |
| | | immunoglobulin-like | | | | | |
| | | receptor 3DL2 | | | | | |
| 67 | N | Peroxiredoxin-1 | Peroxiredoxin-1 | Q06830 | 5052 | PRDX1 | 1.262708 |
| 68 | N | Ubiquitin + 1, | Ubiquitin + | P62979 | 6233 | RPS27A | 1.230309 |
| | | truncated mutation | 1 | | | | |
| | | for UbB | | | | | |
| 69 | N | Macrophage colony- | M-CSF R | P07333 | 1436 | CSF1R | 1.12842 |
| | | stimulating factor 1 | | | | | |
| | | receptor | | | | | |
| 70 | N | Lymphocyte | LAG-3 | P18627 | 3902 | LAG3 | 1.090413 |
| | | activation gene 3 | | | | | |
| | | protein | | | | | |
| 71 | N | Inhibitor of growth | ING1 | Q9UK53 | 3621 | ING1 | 1.257664 |
| | | protein 1 | | | | | |
| 72 | N | Cathepsin S | Cathepsin | P25774 | 1520 | CTSS | 1.120017 |
| | | | S | | | | |
| 73 | N | SUMO-conjugating | UBC9 | P63279 | 7329 | UBE2I | 1.277461 |
| | | enzyme UBC9 | | | | | |
| 74 | N | Neurogenic locus | Notch-3 | Q9UM47 | 4854 | NOTCH3 | 1.113985 |
| | | notch homolog | | | | | |
| | | protein 3 | | | | | |
| 75 | N | Angiopoietin-related | ANGL4 | Q9BY76 | 51129 | ANGPTL4 | 1.094448 |
| | | protein 4 | | | | | |
| 76 | N | Granulysin | Granulysin | P22749 | 10578 | GNLY | 1.141104 |
| 77 | N | Pulmonary | SP-D | P35247 | 6441 | SFTPD | 1.150353 |
| | | surfactant-associated | | | | | |
| | | protein D | | | | | |
| 78 | N | Proliferation- | PA2G4 | Q9UQ80 | 5036 | PA2G4 | 1.236745 |
| | | associated protein | | | | | |
| | | 2G4 | | | | | |
| 79 | N | Copine-1 | CPNE1 | Q99829 | 8904 | CPNE1 | 1.192813 |
| 80 | N | Urokinase | suPAR | Q03405 | 5329 | PLAUR | 1.155834 |
| | | plasminogen | | | | | |
| | | activator surface | | | | | |
| | | receptor | | | | | |
| 81 | N | Dual specificity | MP2K2 | P36507 | 5605 | MAP2K2 | 1.157307 |
| | | mitogen-activated | | | | | |
| | | protein kinase kinase | | | | | |
| | | 2 | | | | | |
| 82 | N | Histidine-rich | HRG | P04196 | 3273 | HRG | 1.117755 |
| | | glycoprotein | | | | | |
| 83 | N | Neuroligin-4, | NLGNX | Q8N0W4 | 57502 | NLGN4X | 1.07877 |
| | | X-linked | | | | | |
| 84 | N | Carbonic anhydrase 3 | Carbonic | P07451 | 761 | CA3 | 1.441555 |
| | | | anhydrase | | | | |
| | | | III | | | | |
| 85 | N | Histidine triad | HINT1 | P49773 | 3094 | HINT1 | 1.191901 |
| | | nucleotide-binding | | | | | |
| | | protein 1 | | | | | |
| 86 | N | C-X-C motif | I-TAC | O14625 | 6373 | CXCL11 | 1.277508 |
| | | chemokine 11 | | | | | |
| 87 | N | Calcium/calmodulin- | CAMK2D | Q13557 | 817 | CAMK2D | 1.400521 |
| | | dependent protein | | | | | |
| | | kinase type II subunit | | | | | |
| | | delta | | | | | |
| 88 | N | Retinoic acid | TIG2 | Q99969 | 5919 | RARRES2 | 1.111269 |
| | | receptor responder | | | | | |
| | | protein 2 | | | | | |
| 89 | N | Protein kinase C | KPCT | Q04759 | 5588 | PRKCQ | 1.37128 |
| | | theta type | | | | | |
| 90 | N | NKG2D ligand 1 | ULBP-1 | Q9BZM | 80329 | ULBP1 | 1.275763 |
| | | | | 6 | | | |
| 91 | N | Macrophage | MIF | P14174 | 4282 | MIF | 1.045245 |
| | | migration inhibitory | | | | | |
| | | factor | | | | | |
| 92 | N | importin subunit | Karyopherin- | P52292 | 3838 | KPNA2 | 1.261797 |
| | | alpha-1 | a2 | | | | |
| 93 | N | Hepatitis A virus | TIMD3 | Q8TDQ0 | 84868 | HAVCR2 | 1.117355 |
| | | cellular receptor 2 | | | | | |
| 94 | N | cAMP-regulated | ARP19 | P56211 | 10776 | ARPP19 | 1.257916 |
| | | phosphoprotein 19 | | | | | |
| 95 | N | Stress-induced- | Stress-induced- | P31948 | 10963 | STIP1 | 1.161618 |
| | | phosphoprotein 1 | phosphoprotein 1 | | | | |
| 96 | N | Complement decay- | DAF | P08174 | 1604 | CD55 | 1.088055 |
| | | accelerating factor | | | | | |
| 97 | N | Dickkopf-related | DKK3 | Q9UBP4 | 27122 | DKK3 | 1.138268 |
| | | protein 3 | | | | | |
| 98 | N | Mitogen-activated | MK08 | P45983 | 5599 | MAPK8 | 1.211317 |
| | | protein kinase 8 | | | | | |
| 99 | Y | Junctional adhesion | JAM-B | P57087 | 58494 | JAM2 | 1.069037 |
| | | molecule B | | | | | |
| 100 | N | Translationally- | TCTP | P13693 | 7178 | TPT1 | 1.284694 |
| | | controlled tumor | | | | | |
| | | protein | | | | | |
| 101 | N | Kin of IRRE-like | KIRR3 | Q8IZU9 | 84623 | KIRREL3 | 1.016634 |
| | | protein 3 | | | | | |
| 102 | N | Neurexin-3-beta | NRX3B | Q9HDB5 | 9369 | NRXN3 | 1.134604 |
| 103 | N | Phosphatidylethanol | prostatic | P30086 | 5037 | PEBP1 | 1.223864 |
| | | amine-binding | binding | | | | |
| | | protein 1 | protein | | | | |

TABLE 2
Exemplary biomarkers with decreased expression from proteome analysis
of individuals with Down syndrome compared to typical individuals.

| | Chr | | | | Ertirez Gene | EntrezGene | fold |
|---|---|---|---|---|---|---|---|
| | 21 | Target Full Name | Target | UniProt | ID | Symbol | change |
| 1 | N | Noggin | Noggin | Q13253 | 9241 | NOG | 0.675275 |
| 2 | N | Thyroxine-binding | Thyroxine- | P05543 | 6906 | SERPINA7 | 0.832652 |
| | | globulin | Binding | | | | |
| | | | Globulin | | | | |
| 3 | N | Antithrombin-III | Antithrombin | P01008 | 462 | SERPINC1 | 0.834312 |
| | | | III | | | | |
| 4 | N | Neurexophilin-1 | NXPH1 | P58417 | 30010 | NXPH1 | 0.730679 |
| 5 | N | Epidermal growth | ERBB1 | P00533 | 1956 | EGFR | 0.810019 |
| | | factor receptor | | | | | |
| 6 | N | Coagulation Factor X | Coagulation | P00742 | 2159 | F10 | 0.834765 |
| | | | Factor X | | | | |
| 7 | N | Thrombopoietin | Thrombopoietin | P40238 | 4352 | MPL | 0.881477 |
| | | Receptor | Receptor | | | | |
| 8 | N | Vascular endothelial | VEGF sR2 | P35968 | 3791 | KDR | 0.764755 |
| | | growth factor | | | | | |
| | | receptor 2 | | | | | |
| 9 | N | Heparan-sulfate 6-O- | H6ST1 | O60243 | 9394 | HS6ST1 | 0.807388 |
| | | sulfotransferase 1 | | | | | |
| 10 | N | Properdin | Properdin | P27918 | 5199 | CFP | 0.819579 |
| 11 | N | Cardiotrophin-1 | Cardiotrophin-1 | Q16619 | 1489 | CTF1 | 0.891954 |
| 12 | N | Fc receptor-like | FCRL3 | Q96P31 | 115352 | FCRL3 | 0.821431 |
| | | protein 3 | | | | | |
| 13 | N | Vitamin K-dependent | Protein S | P07225 | 5627 | PROS1 | 0.858111 |
| | | protein S | | | | | |
| 14 | N | Alpha-1- | a1- | P01011 | 12 | SERPINA3 | 0.882378 |
| | | antichymotrypsin | Antichymotrypsin | | | | |
| 15 | N | Receptor tyrosine- | ERBB3 | P21860 | 2065 | ERBB3 | 0.813753 |
| | | protein kinase erbB-3 | | | | | |
| 16 | N | Complement factor H | Factor H | P08603 | 3075 | CFH | 0.911609 |
| 17 | N | Ficolin-2 | FCN2 | Q15485 | 2220 | FCN2 | 0.843073 |
| 18 | N | Sialic acid-binding Ig- | Siglec-7 | Q9Y286 | 27036 | SIGLEC7 | 0.858193 |
| | | like lectin 7 | | | | | |
| 19 | N | Prothrombin | Prothrombin | P00734 | 2147 | F2 | 0.873152 |
| 20 | N | Apolipoprotein E | Apo E2 | P02649 | 348 | APOE | 0.891094 |
| | | (isoform E2) | | | | | |
| 21 | N | Tyrosine-protein | Dtk | Q06418 | 7301 | TYRO3 | 0.874194 |
| | | kinase receptor | | | | | |
| | | TYRO3 | | | | | |
| 22 | N | Alpha-2- | a2- | P01023 | 2 | A2M | 0.690918 |
| | | macroglobulin | Macroglobulin | | | | |
| 23 | N | Complement C1r | C1r | P00736 | 715 | C1R | 0.651911 |
| | | subcomponent | | | | | |
| 24 | N | Pappalysin-1 | PAPP-A | Q13219 | 5069 | PAPPA | 0.674607 |
| 25 | N | Leukocyte | ILT-2 | Q8NHL6 | 10859 | LILRB1 | 0.792961 |
| | | immunoglobulin-like | | | | | |
| | | receptor subfamily B | | | | | |
| | | member 1 | | | | | |
| 26 | N | Leukotriene A-4 | LKHA4 | P09960 | 4048 | LTA4H | 0.728379 |
| | | hydrolase | | | | | |
| 27 | N | L-Selectin | sL-Selectin | P14151 | 6402 | SELL | 0.88221 |
| 28 | N | Alpha-2-HS- | a2-HS- | P02765 | 197 | AHSG | 0.873822 |
| | | glycoprotein | Glycoprotein | | | | |
| 29 | N | A disintegrin and | ATS13 | Q76LX8 | 11093 | ADAMTS13 | 0.734796 |
| | | metalloproteinase | | | | | |
| | | with | | | | | |
| | | thrombospondin | | | | | |
| | | motifs 13 | | | | | |
| 30 | N | Tartrate-resistant | TrATPase | P13686 | 54 | ACP5 | 0.797884 |
| | | acid phosphatase | | | | | |
| | | type 5 | | | | | |
| 31 | N | Apolipoprotein B | Apo B | P04114 | 338 | APOB | 0.694715 |
| 32 | N | Neutral ceramidase | ASAH2 | Q9NR71 | 56624 | ASAH2 | 0.776302 |
| 33 | N | Proprotein | PCSK7 | Q16549 | 9159 | PCSK7 | 0.740934 |
| | | convertase | | | | | |
| | | subtilisin/kexin type | | | | | |
| | | 7 | | | | | |
| 34 | N | NT-3 growth factor | TrkC | Q16288 | 4916 | NTRK3 | 0.710201 |
| | | receptor | | | | | |
| 35 | N | Receptor tyrosine- | ERBB4 | Q15303 | 2066 | ERBB4 | 0.906246 |
| | | protein kinase erbB-4 | | | | | |
| 36 | N | Tumor necrosis | CD30 | P32971 | 944 | TNFSF8 | 0.908843 |
| | | factor ligand | Ligand | | | | |
| | | superfamily member | | | | | |
| | | 8 | | | | | |
| 37 | N | BDNF/NT-3 growth | TrkB | Q16620 | 4915 | NTRK2 | 0.861473 |
| | | factors receptor | | | | | |
| 38 | N | Tumor necrosis | CD30 | P28908 | 943 | TNFRSF8 | 0.915738 |
| | | factor receptor | | | | | |
| | | superfamily member | | | | | |
| | | 8 | | | | | |
| 39 | N | Proto-oncogene | RET | P07949 | 5979 | RET | 0.819819 |
| | | tyrosine-protein | | | | | |
| | | kinase receptor Ret | | | | | |
| 40 | N | Protein FAM107B | FAM107B | Q9H098 | 83641 | FAM107B | 0.934452 |
| 41 | N | Immunoglobulin E | IgE | P01854 | 3497 50802 | IGHE IGK@ | 0.283216 |
| | | | | | 3535 | IGL@ | |
| 42 | N | Coagulation factor Xa | Coagulation | P00742 | 2159 | F10 | 0.846881 |
| | | | Factor Xa | | | | |
| 43 | N | Interleukin-20 | IL-20 | Q9NYY1 | 50604 | IL20 | 0.910328 |
| 44 | N | Cadherin-3 | P- | P22223 | 1001 | CDH3 | 0.883997 |
| | | | Cadherin | | | | |
| 45 | N | Complement C3 | C3 | P01024 | 718 | C3 | 0.889236 |
| 46 | N | Platelet-activating | PAFAH | Q13093 | 7941 | PLA2G7 | 0.749241 |
| | | factor | | | | | |
| | | acetylhydrolase | | | | | |
| 47 | N | Apolipoprotein E | Apo E3 | P02649 | 348 | APOE | 0.87908 |
| | | (isoform E3) | | | | | |
| 48 | N | Apolipoprotein E | Apo E4 | P02649 | 348 | APOE | 0.882155 |
| | | (isoform E4) | | | | | |
| 49 | N | Dickkopf-related | Dkk-4 | Q9UBT3 | 27121 | DKK4 | 0.751078 |
| | | protein 4 | | | | | |
| 50 | N | Lactoperoxidase | PERL | P22079 | 4025 | LPO | 0.631003 |
| 51 | N | Glypican-5 | GPC5 | P78333 | 2262 | GPC5 | 0.958825 |
| 52 | N | Dickkopf-related | DKK1 | O94907 | 22943 | DKK1 | 0.726191 |
| | | protein 1 | | | | | |
| 53 | N | Dipeptidyl peptidase | CATC | P53634 | 1075 | CTSC | 0.920218 |
| | | 1 | | | | | |
| 54 | N | Oncostatin-M | OSM | P13725 | 5008 | OSM | 0.863759 |
| 55 | N | Plexin-C1 | PLXC1 | O60486 | 10154 | PLXNC1 | 0.875611 |
| 56 | N | Fibroblast growth | FGF-19 | O95750 | 9965 | FGF19 | 0.68713 |
| | | factor 19 | | | | | |
| 57 | N | T-lymphocyte | B7 | P33681 | 941 | CD80 | 0.90921 |
| | | activation antigen | | | | | |
| | | CD80 | | | | | |
| 58 | N | Fibrinogen | Fibrinogen | P02671 | 2243 2244 | FGA FGB | 0.917282 |
| | | | | P02675 | 2266 | FGG | |
| | | | | P02679 | | | |
| 59 | N | Tumor necrosis | OPG | O00300 | 4982 | TNFRSF11B | 0.831605 |
| | | factor receptor | | | | | |
| | | superfamily member | | | | | |
| | | 11B | | | | | |
| 60 | N | Lipopolysaccharide- | LBP | P18428 | 3929 | LBP | 0.909365 |
| | | binding protein | | | | | |
| 61 | N | Apolipoprotein A-I | Apo A-I | P02647 | 335 | APOA1 | 0.818103 |
| 62 | N | Stabilin-2 | STAB2 | Q8WW | 55576 | STAB2 | 0.89358 |
| | | | | Q8 | | | |
| 63 | N | Carbonic anhydrase 6 | Carbonic | P23280 | 765 | CA6 | 0.641303 |
| | | | anhydrase | | | | |
| | | | 6 | | | | |
| 64 | N | Glypican-3 | Glypican 3 | P51654 | 2719 | GPC3 | 0.782954 |
| 65 | N | Prostate-specific | PSA | P07288 | 354 | KLK3 | 0.922078 |
| | | antigen | | | | | |
| 66 | N | Neurogenic locus | Notch 1 | P46531 | 4851 | NOTCH1 | 0.847085 |
| | | notch homolog | | | | | |
| | | protein 1 | | | | | |
| 67 | N | Platelet-derived | PDGF Rb | P09619 | 5159 | PDGFRB | 0.785868 |
| | | growth factor | | | | | |
| | | receptor beta | | | | | |
| 68 | N | Mitogen-activated | JNK2 | P45984 | 5601 | MAPK9 | 0.89164 |
| | | protein kinase 9 | | | | | |
| 69 | N | Mast/stem cell | SCF sR | P10721 | 3815 | KIT | 0.791132 |
| | | growth factor | | | | | |
| | | receptor Kit | | | | | |
| 70 | N | Creatine kinase M- | CK-MM | P06732 | 1158 | CKM | 0.798072 |
| | | type | | | | | |
| 71 | N | Insulin-like growth | IGFBP-3 | P17936 | 3486 | IGFBP3 | 0.798641 |
| | | factor-binding | | | | | |
| | | protein 3 | | | | | |
| 72 | N | Thrombin | Thrombin | P00734 | 2147 | F2 | 0.657699 |
| 73 | N | C-C motif chemokine | I-309 | P22362 | 6346 | CCL1 | 0.84269 |
| | | 1 | | | | | |
| 74 | N | Contactin-4 | Contactin-4 | Q8IWV2 | 152330 | CNTN4 | 0.844185 |
| 75 | N | C-C motif chemokine | LD78-beta | P16619 | 414062 | CCL3L1 | 0.887834 |
| | | 3-like 1 | | | | | |
| 76 | N | Serotransferrin | Transferrin | P02787 | 7018 | TF | 0.892147 |
| 77 | N | Carbonic anhydrase 4 | Carbonic | P22748 | 762 | CA4 | 0.837595 |
| | | | Anhydrase | | | | |
| | | | IV | | | | |
| 78 | N | Leukocyte | ILT-4 | Q8N423 | 10288 | LILRB2 | 0.798373 |
| | | immunoglobulin-like | | | | | |
| | | receptor subfamily B | | | | | |
| | | member 2 | | | | | |
| 79 | N | SPARC | ON | P09486 | 6678 | SPARC | 0.71233 |
| 80 | N | Serine/threonine- | STK16 | O75716 | 8576 | STK16 | 0.888105 |
| | | protein kinase 16 | | | | | |
| 81 | N | Lysosomal protective | Cathepsin | P10619 | 5476 | CTSA | 0.814946 |
| | | protein | A | | | | |
| 82 | N | Intercellular | sICAM-2 | P13598 | 3384 | ICAM2 | 0.797588 |
| | | adhesion molecule 2 | | | | | |
| 83 | N | Complement C1s | C1s | P09871 | 716 | C1S | 0.924824 |
| | | subcomponent | | | | | |
| 84 | N | Kallistatin | Kallistatin | P29622 | 5267 | SERPINA4 | 0.888752 |
| 85 | N | Polymeric | PIGR | P01833 | 5284 | PIGR | 0.810904 |
| | | immunoglobulin | | | | | |
| | | receptor | | | | | |
| 86 | N | Platelet glycoprotein | CD36 | P16671 | 948 | CD36 | 0.80299 |
| | | 4 | ANTIGEN | | | | |
| 87 | N | 26S proteasome non- | PSD7 | P51665 | 5713 | PSMD7 | 0.929881 |
| | | ATPase regulatory | | | | | |
| | | subunit 7 | | | | | |
| 88 | N | Roundabout | ROBO2 | Q9HCK4 | 6092 | ROBO2 | 0.848573 |
| | | homolog 2 | | | | | |
| 89 | N | Complement | C6 | P13671 | 729 | C6 | 0.893219 |
| | | component C6 | | | | | |
| 90 | N | Kallikrein-5 | kallikrein | Q9Y337 | 25818 | KLK5 | 0.95128 |
| | | | 5 | | | | |
| 91 | N | Angiopoietin-1 | Angiopoietin-1 | Q15389 | 284 | ANGPT1 | 0.791938 |
| 92 | N | Cytoskeleton- | CKAP2 | Q8WWK9 | 26586 | CKAP2 | 0.906919 |
| | | associated protein 2 | | | | | |
| 93 | N | High affinity | FCGR1 | P12314 | 2209 | FCGR1A | 0.895982 |
| | | immunoglobulin | | | | | |
| | | gamma Fc receptor I | | | | | |
| 94 | N | Serum | paraoxonase 1 | P27169 | 5444 | PON1 | 0.906718 |
| | | paraoxonase/ | | | | | |
| | | arylesterase 1 | | | | | |
| 95 | N | Complement C1q | C1q | P02745 | 712 713 714 | C1QA C1QB | 0.896634 |
| | | subcomponent | | P02746 | | C1QC | |
| | | | | P02747 | | | |
| 96 | N | Immunoglobulin M | IgM | P01871 | 3507 3512 | IGHM IGJ | 0.784602 |
| | | | | | 50802 3535 | IGK@ IGL@ | |
| 97 | N | Carbonic anhydrase 7 | Carbonic | P43166 | 766 | CA7 | 0.841378 |
| | | | anhydrase | | | | |
| | | | VII | | | | |
| 98 | N | Creatine kinase M- | CK-MB | P12277 | 1152 1158 | CKB CKM | 0.659184 |
| | | type: Creatine kinase | | P06732 | | | |
| | | B-type heterodimer | | | | | |
| 99 | N | Glypican-2 | GPC2 | Q8N158 | 221914 | GPC2 | 0.970699 |
| 100 | N | Chromobox protein | CBX5 | P45973 | 23468 | CBX5 | 0.92104 |
| | | homolog 5 | | | | | |
| 101 | N | Leucine-rich repeats | LRIG3 | Q6UXM1 | 121227 | LRIG3 | 0.835739 |
| | | and immunoglobulin- | | | | | |
| | | like domains protein | | | | | |
| | | 3 | | | | | |
| 102 | N | C-C motif chemokine | MCP-2 | P80075 | 6355 | CCL8 | 0.922648 |
| | | 8 | | | | | |
| 103 | N | Calpastatin | Calpastatin | P20810 | 831 | CAST | 0.892644 |
| 104 | N | Epithelial discoidin | discoidin | Q08345 | 780 | DDR1 | 0.871296 |
| | | domain-containing | domain | | | | |
| | | receptor 1 | receptor 1 | | | | |
| 105 | N | Desmoglein-1 | Desmoglein-1 | Q02413 | 1828 | DSG1 | 0.89153 |
| 106 | N | Lymphotoxin | Lymphotoxin | P01374, | 4049 4050 | LTA LTB | 0.896966 |
| | | alpha1: beta2 | a1/b2 | Q06643 | | | |
| 107 | N | Prolyl endopeptidase | SEPR | Q12884 | 2191 | FAP | 0.804815 |
| | | FAP | | | | | |
| 108 | N | Persephin | Persephin | O60542 | 5623 | PSPN | 0.935024 |
| 109 | N | C3a anaphylatoxin | C3adesArg | P01024 | 718 | C3 | 0.891053 |
| | | des Arginine | | | | | |
| 110 | N | Neural cell adhesion | NCAM- | P13591 | 4684 | NCAM1 | 0.850319 |
| | | molecule 1, 120 kDa | 120 | | | | |
| | | isoform | | | | | |
| 111 | N | B-cell receptor CD22 | CD22 | P20273 | 933 | CD22 | 0.860088 |
| 112 | N | Cyclin-dependent | CDK5/p35 | Q00535 | 1020 8851 | CDK5 | 0.976964 |
| | | kinase 5: Cyclin- | | Q15078 | | CDK5R1 | |
| | | dependent kinase 5 | | | | | |
| | | activator 1 complex | | | | | |
| 113 | N | Complement | C1QBP | Q07021 | 708 | C1QBP | 0.923273 |
| | | component 1 Q | | | | | |
| | | subcomponent- | | | | | |
| | | binding protein, | | | | | |
| | | mitochondrial | | | | | |
| 114 | N | Urokinase-type | uPA | P00749 | 5328 | PLAU | 0.841005 |
| | | plasminogen | | | | | |
| | | activator | | | | | |
| 115 | N | Interleukin-34 | IL-34 | Q6ZMJ4 | 146433 | IL34 | 0.855807 |
| 116 | N | Macrophage- | MSP R | Q04912 | 4486 | MST1R | 0.922942 |
| | | stimulating protein | | | | | |
| | | receptor | | | | | |
| 117 | N | Alpha-2-antiplasmin | a2- | P08697 | 5345 | SERPINF2 | 0.917908 |
| | | | Antiplasmin | | | | |
| 118 | N | Tumor necrosis | TNFSF18 | Q9UNG2 | 8995 | TNFSF18 | 0.937803 |
| | | factor ligand | | | | | |
| | | superfamily member | | | | | |
| | | 18 | | | | | |
| 119 | N | Matrilin-3 | MATN3 | O15232 | 4148 | MATN3 | 0.920835 |
| 120 | N | Cathepsin L2 | Cathepsin | O60911 | 1515 | CTSV | 0.790695 |
| | | | V | | | | |
| 121 | N | Protein E7_HPV18 | HPV E7 | P06788 | 1489089 | Human- | 0.905728 |
| | | | Type18 | | | virus | |
| 122 | N | Thyroglobulin | Thyroglobulin | P01266 | 7038 | TG | 0.93678 |
| 123 | N | Fibronectin Fragment | FN1.4 | P02751 | 2335 | FN1 | 0.850937 |
| | | 4 | | | | | |
| 124 | N | Fibroblast growth | b-ECGF | P05230 | 2246 | FGF1 | 0.935531 |
| | | factor 1 | | | | | |
| 125 | N | Serine/threonine- | CHK1 | O14757 | 1111 | CHEK1 | 0.927251 |
| | | protein kinase Chk1 | | | | | |
| 126 | N | Protein NOV | NovH | P48745 | 4856 | NOV | 0.966955 |
| | | homolog | | | | | |
| 127 | N | Kallikrein-6 | Kailikrein | Q92876 | 5653 | KLK6 | 0.863278 |
| | | | 6 | | | | |
| 128 | N | SLAM family member | SLAF6 | Q96DU3 | 114836 | SLAMF6 | 0.819122 |
| | | 6 | | | | | |
| 129 | N | Histone H1.2 | Histone | P16403 | 3006 | HIST1H1C | 0.87997 |
| | | | H1.2 | | | | |
| 130 | N | Tyrosine-protein | ZAP70 | P43403 | 7535 | ZAP70 | 0.823703 |
| | | kinase ZAP-70 | | | | | |
| 131 | N | Cystatin-D | CYTD | P28325 | 1473 | CST5 | 0.60898 |
| 132 | N | Cystatin-SA | CYTT | P09228 | 1470 | CST2 | 0.778701 |
| 133 | N | Interleukin-37 | IL-1F7 | Q9NZH6 | 27178 | IL37 | 0.975285 |
| 134 | N | DNA repair protein | RAD51 | Q06609 | 5888 | RAD51 | 0.854443 |
| | | RAD51 homolog 1 | | | | | |
| 135 | N | Carbohydrate | CHST6 | Q9GZX3 | 4166 | CHST6 | 0.965035 |
| | | sulfotransferase 6 | | | | | |
| 136 | N | Bone morphogenetic | BMP RII | Q13873 | 659 | BMPR2 | 0.682626 |
| | | protein receptor | | | | | |
| | | type-2 | | | | | |
| 137 | N | Interferon lambda-2 | IFN- | Q8IZJ0 | 282616 | IFNL2 | 0.913529 |
| | | | lambda 2 | | | | |
| 138 | N | Ectonucleoside | CD39 | P49961 | 953 | ENTPD1 | 0.990381 |
| | | triphosphate | | | | | |
| | | diphosphohydrolase | | | | | |
| | | 1 | | | | | |
| 139 | N | Platelet endothelial | PECAM-1 | P16284 | 5175 | PECAM1 | 0.933147 |
| | | cell adhesion | | | | | |
| | | molecule | | | | | |
| 140 | N | Endoglin | Endoglin | P17813 | 2022 | ENG | 0.727157 |
| 141 | N | Homeobox protein | NANOG | Q9H9S0 | 79923 | NANOG | 0.948341 |
| | | NANOG | | | | | |
| 142 | N | Superoxide | Mn SOD | P04179 | 6648 | SOD2 | 0.907978 |
| | | dismutase [Mn], | | | | | |
| | | mitochondrial | | | | | |
| 143 | N | Renin | Renin | P00797 | 5972 | REN | 0.866755 |
| 144 | N | Angiostatin | Angiostatin | P00747 | 5340 | PLG | 0.908314 |
| 145 | N | Complement | C8 | P07357, | 731 732 733 | C8A C8B | 0.861412 |
| | | component C8 | | P07358, | | C8G | |
| | | | | P07360 | | | |
| 146 | N | Cytokine receptor- | CLF-1/CLC | O75462 | 9244 23529 | CRLF1 CLCF1 | 0.959346 |
| | | like factor | Complex | Q9UBD9 | | | |
| | | 1: Cardiotrophin-like | | | | | |
| | | cytokine factor 1 | | | | | |
| | | Complex | | | | | |
| 147 | N | C-type lectin domain | DC-SIGNR | Q9H2X3 | 10332 | CLEC4M | 0.895613 |
| | | family 4 member M | | | | | |
| 148 | N | CD97 antigen | CD97 | P48960 | 976 | CD97 | 0.876891 |
| 149 | N | Trypsin-3 | TRY3 | P35030 | 5646 | PRSS3 | 0.94095 |
| 150 | N | Tumor necrosis | 4-1BB | P41273 | 8744 | TNFSF9 | 0.987214 |
| | | factor ligand | ligand | | | | |
| | | superfamily member | | | | | |
| | | 9 | | | | | |
| 151 | N | Platelet-derived | PDGF-BB | P01127 | 5155 | PDGFB | 0.764184 |
| | | growth factor | | | | | |
| | | subunit B | | | | | |
| 152 | N | Serine/threonine- | PIM1 | P11309 | 5292 | PIM1 | 0.946344 |
| | | protein kinase pim-1 | | | | | |
| 153 | N | Aurora kinase B | AURKB | Q96GD4 | 9212 | AURKB | 0.937148 |
| 154 | N | Group 10 secretory | GX | O15496 | 8399 | PLA2G10 | 0.973131 |
| | | phospholipase A2 | | | | | |
| 155 | N | Peptidoglycan | PGRP-S | O75594 | 8993 | PGLYRP1 | 0.472692 |
| | | recognition protein 1 | | | | | |
| 156 | N | Bone morphogenetic | BMP-7 | P18075 | 655 | BMP7 | 0.935364 |
| | | protein 7 | | | | | |
| 157 | N | Lymphotactin | Lymphotactin | P47992 | 6375 | XCL1 | 0.974522 |
| 158 | N | Glutamate | PSMA | Q04609 | 2346 | FOLH1 | 0.901196 |
| | | carboxypeptidase 2 | | | | | |
| 159 | N | Sonic hedgehog | Sonic | Q15465 | 6469 | SHH | 0.843662 |
| | | protein | Hedgehog | | | | |
| 160 | N | Angiopoietin-1 | sTie-2 | Q02763 | 7010 | TEK | 0.940492 |
| | | receptor, soluble | | | | | |
| 161 | N | Cysteine-rich | CRIS3 | P54108 | 10321 | CRISP3 | 0.944708 |
| | | secretory protein 3 | | | | | |
| 162 | N | NADPH-cytochrome | NADPH- | P16435 | 5447 | POR | 0.836957 |
| | | P450 reductase | P450 | | | | |
| | | | Oxidoreductase | | | | |
| 163 | N | Proteasome subunit | PSA2 | P25787 | 5683 | PSMA2 | 0.812429 |
| | | alpha type-2 | | | | | |
| 164 | N | Alkaline | Alkaline | P05186 | 249 | ALPL | 0.894702 |
| | | phosphatase, tissue- | phosphatase, | | | | |
| | | nonspecific isozyme | bone | | | | |
| 165 | N | C-X-C motif | ENA-78 | P42830 | 6374 | CXCL5 | 0.914178 |
| | | chemokine 5 | | | | | |
| 166 | N | Alpha-1- | alpha-1- | P01011 | | SERPINA3 | 0.91008 |
| | | antichymotrypsin | antichymotrypsin | | | | |
| | | complex | complex | | | | |
| 167 | N | Layilin | Layilin | Q6UX15 | 143903 | LAYN | 0.106946 |
| 168 | N | Fractalkine | Fractalkine/ | P78423 | 6376 | CX3CL1 | 0.85759 |
| | | | CX3CL-l | | | | |
| 169 | N | C-C motif chemokine | LAG-1 | Q8NHW4 | 388372 | CCL4L1 | 0.900998 |
| | | 4-like | | | | | |
| 170 | N | Leucine-rich repeat | LRRT1 | Q86UE6 | 347730 | LRRTM1 | 0.946499 |
| | | transmembrane | | | | | |
| | | neuronal protein 1 | | | | | |
| 171 | N | Histone | MOZ | Q92794 | 7994 | KAT6A | 0.93492 |
| | | acetyltransferase | | | | | |
| | | KAT6A | | | | | |
| 172 | N | Disintegrin and | ADAM12 | O43184 | 8038 | ADAM12 | 0.862185 |
| | | metalloproteinase | | | | | |
| | | domain-containing | | | | | |
| | | protein 12 | | | | | |
| 173 | N | Interleukin-17A | IL-17 | Q16552 | 3605 | IL17A | 0.94488 |
| 174 | N | Thymidine kinase, | Thymidine | P04183 | 7083 | TK1 | 0.941196 |
| | | cytosolic | kinase | | | | |
| 175 | N | C-C motif chemokine | 6Ckine | O00585 | 6366 | CCL21 | 0.846855 |
| | | 21 | | | | | |
| 176 | N | Fibroblast growth | FGF-12 | P61328 | 2257 | FGF12 | 0.946963 |
| | | factor 12 | | | | | |
| 177 | N | Heparin cofactor 2 | Heparin | P05546 | 3053 | SERPIND1 | 0.903865 |
| | | | cofactor II | | | | |
| 178 | N | Baculoviral IAP | Livin B | Q96CA5 | 79444 | BIRC7 | 0.93441 |
| | | repeat-containing | | | | | |
| | | protein 7 Isoform | | | | | |
| | | beta | | | | | |
| 179 | N | Netrin-4 | NET4 | Q9HB63 | 59277 | NTN4 | 0.915557 |
| 180 | N | Ck-beta-8-1 | Ck-b-8-1 | P55773 | 6368 | CCL23 | 0.775992 |
| 181 | N | Ciliary neurotrophic | CNTF | P26441 | 1270 | CNTF | 0.977592 |
| | | factor | | | | | |
| 182 | N | Lysozyme C | Lysozyme | P61626 | 4069 | LYZ | 0.892148 |
| 183 | N | C-C motif chemokine | MPIF-1 | P55773 | 6368 | CCL23 | 0.801946 |
| | | 23 | | | | | |
| 184 | N | C-type lectin domain | CLC1B | Q9P126 | 51266 | CLEC1B | 0.784385 |
| | | family 1 member B | | | | | |
| 185 | N | Prefoldin subunit 5 | PFD5 | Q99471 | 5204 | PFDN5 | 0.854327 |
| 186 | N | Tumor necrosis | sRANKL | O14788 | 8600 | TNFSF11 | 0.873328 |
| | | factor ligand | | | | | |
| | | superfamily member | | | | | |
| | | 11 | | | | | |
| 187 | N | Cystatin-SN | CYTN | P01037 | 1469 | CST1 | 0.815877 |
| 188 | N | Tumor necrosis | LIGHT | O43557 | 8740 | TNFSF14 | 0.8943 |
| | | factor ligand | | | | | |
| | | superfamily member | | | | | |
| | | 14 | | | | | |
| 189 | N | Serine/threonine- | PAK3 | O75914 | 5063 | PAK3 | 0.888912 |
| | | protein kinase PAK 3 | | | | | |
| 190 | N | Cadherin-5 | Cadherin- | P33151 | 1003 | CDH5 | 0.878675 |
| | | | 5 | | | | |
| 191 | N | Granzyme H | Granzyme | P20718 | 2999 | GZMH | 0.965973 |
| | | | H | | | | |
| 192 | N | Interferon alpha-2 | IFN-aA | P01563 | 3440 | IFNA2 | 0.933058 |
| 193 | N | Interleukin-1 | IL-1 R AcP | Q9NPH3 | 3556 | IL1RAP | 0.900584 |
| | | Receptor accessory | | | | | |
| | | protein | | | | | |
| 194 | N | Mannose-binding | MBL | P11226 | 4153 | MBL2 | 0.779276 |
| | | protein C | | | | | |
| 195 | N | Tumor necrosis | Lymphotoxin | P36941 | 4055 | LTBR | 0.882008 |
| | | factor receptor | b R | | | | |
| | | superfamily member | | | | | |
| | | 3 | | | | | |
| 196 | N | Glia-derived nexin | Protease | P07093 | 5270 | SERPINE2 | 0.857869 |
| | | | nexin I | | | | |
| 197 | N | Artemin | Artemin | Q5T4W7 | 9048 | ARTN | 0.923443 |
| 198 | N | Interleukin-23 | IL-23 | P29460, | 3593 51561 | IL12B IL23A | 0.964813 |
| | | | | Q9NPF7 | | | |
| 199 | N | Protein Rev_HV2BE | HIV-2 Rev | P18093 | 1724716 | Human- | 0.966873 |
| | | | | | | virus | |
| 200 | N | Platelet-derived | PDGF-AA | P04085 | 5154 | PDGFA | 0.825316 |
| | | growth factor | | | | | |
| | | subunit A | | | | | |
| 201 | N | T-cell surface | sCD4 | P01730 | 920 | CD4 | 0.987444 |
| | | glycoprotein CD4 | | | | | |
| 202 | N | Biglycan | BGN | P21810 | 633 | BGN | 0.772285 |
| 203 | N | Leucine-rich repeat | LRRT3 | Q86VH5 | 347731 | LRRTM3 | 0.996228 |
| | | transmembrane | | | | | |
| | | neuronal protein 3 | | | | | |
| 204 | N | Stromelysin-1 | MMP-3 | P08254 | 4314 | MMP3 | 0.891317 |
| 205 | N | Complement | C7 | P10643 | 730 | C7 | 0.932623 |
| | | component C7 | | | | | |
| 206 | N | Afamin | Afamin | P43652 | 173 | AFM | 0.915272 |
| 207 | N | Plasma kallikrein | Prekallikrein | P03952 | 3818 | KLKB1 | 0.932733 |
| 208 | N | Semaphorin-6A | Semaphorin- | Q9H2E6 | 57556 | SEMA6A | 0.77127 |
| | | | 6A | | | | |
| 209 | N | Serum albumin | Albumin | P02768 | 213 | ALB | 0.925506 |
| 210 | N | Receptor-type | Flt-3 | P36888 | 2322 | FLT3 | 0.927655 |
| | | tyrosine-protein | | | | | |
| | | kinase FLT3 | | | | | |
| 211 | N | Granulins | GRN | P28799 | 2896 | GRN | 0.88383 |
| 212 | N | CD166 antigen | ALCAM | Q13740 | 214 | ALCAM | 0.916855 |
| 213 | N | C-C motif chemokine | Eotaxin-2 | O00175 | 6369 | CCL24 | 0.986342 |
| | | 24 | | | | | |
| 214 | N | Baculoviral IAP | Survivin | O15392 | 332 | BIRC5 | 0.952856 |
| | | repeat-containing | | | | | |
| | | protein 5 | | | | | |
| 215 | N | Kallikrein-12 | kallikrein | Q9UKR0 | 43849 | KLK12 | 0.881919 |
| | | | 12 | | | | |
| 216 | N | Muellerian-inhibiting | MIS | P03971 | 268 | AMH | 0.941293 |
| | | factor | | | | | |
| 217 | N | Tyrosine-protein | ABL1 | P00519 | 25 | ABL1 | 0.969493 |
| | | kinase ABL1 | | | | | |
| 218 | N | Sialoadhesin | Sialoadhesin | Q9BZZ2 | 6614 | SIGLEC1 | 0.93097 |
| 219 | N | Wnt inhibitory factor | WIF-1 | Q9Y5W5 | 11197 | WIF1 | 0.901777 |
| | | 1 | | | | | |
| 220 | N | Inducible T-cell | ICOS | Q9Y6W8 | 29851 | ICOS | 0.853916 |
| | | costimulator | | | | | |
| 221 | N | TATA-box-binding | TBP | P20226 | 6908 | TBP | 0.952252 |
| | | protein | | | | | |
| 222 | N | Kallikrein-4 | Kallikrein | Q9Y5K2 | 9622 | KLK4 | 0.960449 |
| | | | 4 | | | | |
| 223 | N | Repulsive guidance | RGMA | Q96B86 | 56963 | RGMA | 0.920558 |
| | | molecule A | | | | | |
| 224 | N | Testican-2 | Testican-2 | Q92563 | 9806 | SPOCK2 | 0.867115 |
| 225 | N | Acidic leucine-rich | AN32B | Q92688 | 10541 | ANP32B | 0.950951 |
| | | nuclear | | | | | |
| | | phosphoprotein 32 | | | | | |
| | | family member B | | | | | |
| 226 | N | Platelet glycoprotein | GP1BA | P07359 | 2811 | GP1BA | 0.91994 |
| | | Ib alpha chain | | | | | |
| 227 | N | Ciliary neurotrophic | CNTFR | P26992 | 1271 | CNTFR | 0.941158 |
| | | factor receptor | alpha | | | | |
| | | subunit alpha | | | | | |
| 228 | N | C-X-C motif | CXCL16, | Q9H2A7 | 58191 | CXCL16 | 0.918026 |
| | | chemokine 16 | soluble | | | | |
| 229 | N | Tumor necrosis | 4-1BB | Q07011 | 3604 | TNFRSF9 | 0.969821 |
| | | factor receptor | | | | | |
| | | superfamily member | | | | | |
| | | 9 | | | | | |
| 230 | N | Neurotrophin-4 | Neurotrophin-5 | P34130 | 4909 | NTF4 | 0.968316 |
| 231 | N | Interleukin-17F | IL-17F | Q96PD4 | 112744 | IL17F | 0.912558 |
| 232 | N | Low affinity | CD23 | P06734 | 2208 | FCER2 | 0.877333 |
| | | immunoglobulin | | | | | |
| | | epsilon Fc receptor | | | | | |
| 233 | N | Transforming growth | TGF-b2 | P61812 | 7042 | TGFB2 | 0.981942 |
| | | factor beta-2 | | | | | |
| 234 | N | Lymphotoxin | Lymphotoxin | P01374, | 4049 4050 | LTA LTB | 0.892884 |
| | | alpha2: beta1 | a2/b1 | Q06643 | | | |
| 235 | N | C-C motif chemokine | MDC | O00626 | 6367 | CCL22 | 0.862217 |
| | | 22 | | | | | |

The foregoing detailed description has been given for clearness of understanding only and no unnecessary limitations should be understood there from as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

The disclosures, including the claims, figures and/or drawings, of each and every patent, patent application, and publication cited herein are hereby incorporated herein by reference in their entireties.

What is claimed is:

1. A method of evaluating a condition or disease prevalent in an individual with Down syndrome compared to a typical individual, comprising:
(a) measuring the level of at least one biomarker and/or the ratio of at least two biomarkers listed in Table 1 or Table 2 in a biological sample obtained from the individual with Down syndrome; and
(b) comparing the measured level or ratio to a reference value or range of reference values, wherein the reference is one or more typical individuals, and wherein a change in the level of the at least one biomarker or the ratio of at least two biomarkers is indicative of the condition or disease prevalent in the individual with Down syndrome.

2. The method of claim 1, wherein the at least one biomarker is a protein, peptide, polypeptide, polynucleotide, transcript, small molecule or microbiome profile.

3. The method of claim 1, wherein the at least one biomarker is a surrogate biomarker.

4. The method of claim 1 , wherein the biological sample is selected from the group consisting of saliva, tears, buccal swabs, nasal epithelium, skin, plasma, urine, blood and stool.

5. The method of claim 4, wherein the biological sample is blood.

6. The method 01 claim 1, wherein the at least one biomarker is selected from Table 1 or Table 2.

7. The method of claim 1, wherein the at least one biomarker is fibroblast growth factor receptor, epidermal growth factor receptor, platelet-derived growth factor receptor, neuropilin, matrix metalloproteinase, B2-microglobulin, Cystatin C, Annexin II, Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin.

8. The method of claim 1, wherein the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSFIB), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III). MBL2, F10 (Factor Xa), BMP7, NOG, DKK1, DKK4, SHH, TFF3, TFF1, Col18A1, TrkB (NTRK2), TrkC (NTRK3) GFBP6 or IgE.

9. The method of claim 1, wherein the at least one biomarker functions in a pathway selected from the group consisting of acute phase response signaling, the complement pathway and the prothrombin pathway.

10. The method of claim 9, wherein the at least one biomarker functions in the acute phase response signaling.

11. The method of claim 10, wherein the at least one biomarker is TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9) or 4-1 BB ligand (TNFSF9).

12. The method of claim 9, wherein the at least one biomarker functions in the complement pathway.

13. The method of claim 12, wherein the at least one biomarker is C1QBP, C1R, C1S, C3, C6, C7, CFH or CFP.

14. The method of claim 9, wherein the at least one biomarker functions in the prothrombin pathway.

15. The method of claim 14, wherein the at least one biomarker is SerpinC1 (antithrombin III), MBL2 or F10 (Factor Xa).

16. The method of claim 1, wherein the measured level of the at least one biomarker is indicative of the type and/or the severity of the condition or disease in the individual with Down syndrome.

17. The method of claim 1, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea

18. A method of evaluating the therapeutic efficacy of a therapeutic intervention for treating a condition or disease in an individual with Down syndrome comprising:
(a) obtaining at least one initial biological sample from the individual at an initial time point, wherein the initial time point is prior to the administration of the therapeutic intervention;
(b) obtaining at least one subsequent biological sample from the individual at a subsequent time point, wherein the subsequent time point is after the administration of the therapeutic intervention;
(c) measuring the level of at least one biomarker listed in Table 1 or Table 2 in the initial and subsequent biological samples; and
(d) comparing the level of the at least one biomarker in the at least one initial biological sample to the level of the at least one biomarker in the at least one subsequent biological sample, wherein a change in the level of the at least one biomarker is indicative of the efficacy of the therapeutic intervention as a treatment for the condition or disease in the individual with Down syndrome.

19. The method of claim 18, wherein the at least one biomarker is a peptide, polypeptide, protein, polynucleotide, transcript, small molecule or microbiome profile.

20. The method of claim 18, wherein the at least one biomarker is a surrogate marker.

21. The method of claim 18, wherein the initial and subsequent biological samples are selected from the group consisting of saliva tears, buccal swab, nasal epithelium, skin, plasma, urine, blood and stool.

22. The method of claim 21, wherein the initial and subsequent biological samples are blood.

23. The method of claim 18, wherein the at least one biomarker is selected from Table 1 or Table 2.

24. The method of claim 18, wherein the at least one biomarker is FGFR1, EGFR, ERBB3, ERBB4, PDGFR, NRP1, MMP1, B2-microglobulin (B2M), Cystatin C (CST3), Annexin H (ANXA2), Notch1, Notch3, TNF sR-1 (TNFRSF1A). TNF sR-2 (TNFRSFIB), CD3O (TNFRSF8), CD30 ligand (TNFSF8), OPG (TNFRSF11B), TAJ (TNFRSF19), DR3 (TNFRSF25), 4-1BB (TNFRSF9), 4-IBB ligand (TNFSF9), IMPDH11MPDF12, C1QBP, C1R. C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE.

25. The method of claim 18, wherein the at least one biomarker functions in a pathway selected from the group consisting of acute phase response signaling, the complement pathway and the prothrombin pathway.

26. The method of claim 18, wherein the at least one biomarker functions in the acute phase response signaling.

27. The method of claim 26, wherein the at least one biomarker is TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9) or 4-1BB ligand (TNFSF9).

28. The method of claim 18, wherein the at least one biomarker functions in the complement pathway.

29. The method of claim 28, wherein the at least one biomarker is C1QBP, C1R, C1S, C3, C6, C7, CFH or CFP.

30. The method of claim 18, wherein the at least one biomarker functions in the prothrombin pathway.

31. The method of claim 30, wherein the at least one biomarker is SerpinC1 (antithrombin III), MBL2 or F10 (Factor Xa).

32. The method of claim 18, wherein the therapeutic intervention is a compound or biologic selected from a compound or biologic library.

33. The method of claim 18, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

34. A method of confirming or refuting a diagnosis of a condition or disease in an individual with Down syndrome comprising:
(a) measuring the level of at least one biomarker and/or the ratio of at least two biomarkers listed in Table 1 or Table 2 in a biological sample obtained from said individual; and
(b) comparing the measured level or ratio to a reference value or range of reference values, wherein the diagnosis of the condition or disease in said individual is confirmed or refuted based on a change in the level of the at least one biomarker or the ratio of at least two biomarkers.

35. The method of claim 34, wherein the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSFIB), CD30 (TNFRSF8), CD30 ligand (TNFSF8), OPG (TNFRSF11B). TAJ (TNFRSF19). DR3 (TNFRSF25), 4-1BB (TNFRSF9), 4-1BB ligand (TNFSF9), IMPDH1, IMPDH2, C1QBP, C1R, C1S, C3. C6, C7, CFR CFP, SerpinC1 (antithrombin III). MBL2, F10 (Factor Xa), BMP7, NOG, DKK1, DKK4, SHH, TFF3, TFF1, Col18A1, TrkB (NTRK2), TrkC (NTRK3), IGFBP6 or IgE.

36. The method of claim 34, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

37. A method of monitoring treatment of a condition or disease in an individual with Down syndrome in need thereof comprising:
(a) obtaining at least one initial biological sample from the individual at an initial time point, wherein the initial time point is prior to the start of a therapeutic intervention protocol for the condition or disease;
(b) obtaining at least one subsequent biological sample from the individual at a subsequent time point, wherein the subsequent time point is after the start of the therapeutic intervention protocol;
(c) measuring the level of at least one biomarker or panel of biomarkers listed in Table 1 or Table 2 in the initial and subsequent biological samples; and
(d) comparing the level of the at least one biomarker or panel of biomarkers in the at least one initial biological sample to the level of the at least one biomarker or panel of biomarkers in the at least one subsequent biological sample, wherein a change in the level of the at least one biomarker or panel of biomarkers is indicative of the efficacy of the therapeutic intervention protocol.

38. The method of claim 37, further comprising modifying or changing the therapeutic intervention protocol based on the level of one or more biomarkers.

39. The method of claim 37, wherein the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSFIA), TNF sR-2 (TNFRSFIB), CD30 (TNFRSF8), CD30 ligand (TNFSF8), OPG (TNFRSF11 B), TAJ (TNFRSF19), DR3 (TNFRSF25), 4-1BB (TNFRSF9), 4-1BB ligand (TNFSF9), IMPDH1, IMPDH2, C1QBP, C1 C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE.

40. The method of claim 37, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

41. A biomarker kit comprising reagents for measuring one or more biomarkers listed in Table 1 or Table 2.

42. The kit of claim 41, wherein the one or more biomarkers is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSFS), CD30 ligand (TNFSF8), OPG (TNFRSF11B), TAJ (TNFRSF19), DR3 (TNFRSF25), 4-1BB (TNFRSF9), 4-IBB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3). IGFBP6 or IgE,

43. The kit of claim 41, wherein the kit further comprises a set of reference values to which the levels of the one or more biomarkers can be compared.

44. The kit of claim 41, wherein the reagents are adapted for measuring biomarkers in a blood sample.

45. The kit of claim 41, wherein the kit further comprises instructions for measuring said one or more biomarkers for diagnosing, evaluating level of severity, or monitoring progression of a condition or disease in an individual with Down syndrome.

46. The kit of claim 41, wherein the kit further comprises instructions for measuring said one or more biomarkers for monitoring the efficacy of a therapeutic intervention in an individual with Down syndrome having a condition or disease.

47. The kit of any one of claim 45 or 46, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea,

48. A method for evaluating a sample from an individual with Down syndrome for a condition or disease, comprising:
preparing a biomarker profile from a biological sample obtained from the individual, and determining the presence or absence of a biomarker signature indicative of the condition or disease, the biomarker profile comprising the level, abundance, or concentration of at least two biomarkers listed in Table 1 or Table 2.

49. The method of claim 48, wherein the at least two biomarkers are selected from the group consisting of FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 and IgE

50. The method of claim 48, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

51. The method of claim 48, wherein the biological sample is a blood sample.

52. A method of evaluating a condition or disease prevalent in a typical individual but rare in an individual with Down syndrome, comprising:
(a) measuring the level of at least one biomarker and/or the ratio of at least two biomarkers listed in Table 1 or Table 2 in a biological sample obtained from the typical individual: and
(b) comparing the measured level or ratio to a reference value or range of reference values, wherein the reference is one or more individuals with Down syndrome, and wherein a change in the level of the at least one biomarker or the ratio of at least two biomarkers is indicative of the condition or disease prevalent in the typical individual.

53. The method of claim 52, wherein the at least one biomarker is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1CBP, C1 C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE.

54. The method of claim 52, wherein the condition or disease comprises heart disease, cancer, stroke, coronary artery disease, atherosclerosis, hypertension and/or angiopathies.

55. A method for treating a condition or disease prevalent in an individual with Down syndrome, the method comprising administering an effective amount of a pharmaceutical composition to the individual, wherein the pharmaceutical composition reduces the expression or activity of a protein in Table 1 associated with the condition or disease prevalent in the individual with Down syndrome.

56. The method of claim 55, wherein the protein is fibroblast growth factor receptor, neuropilin, matrix metalloproteinase, B2-microglobulin, Cystatin C, Annexin II, Notch protein, tumor necrosis factor (TNF) receptor, inosine 5′ monophosphate dehydrogenase, an insulin-like growth factor binding protein, Trefoil factor or endostatin.

57. The method of claim 55, wherein the protein is FGFR1, NRP1, MMP1, B2-microglobulin (B2M), Cystatin C (CST3), Annexin H (ANXA2), Notch3, TNF sR-1 (TNFRSF1A), TNF sR-2 (TNFRSF1B), TAJ (TNFRSFI9), DR3 (TNFRSF25), IMPDH1, IMPDH2, TFF3, TFF1, Col18A1 or IGFBP6.

58. The method of claim 55, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

59. A method for treating a condition or disease prevalent in an individual with Down syndrome, the method comprising administering an effective amount of a pharmaceutical composition to the individual, wherein the pharmaceutical composition increases the expression or activity of a protein in Table 2 associated with the condition or disease prevalent in the individual with Down syndrome.

60. The method of claim 59, wherein the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE.

61. The method of claim 59, wherein the protein is EGFR, ERBB3, ERBB4, PDGFR, Notch1, CD3O (TNFRSF8), CD30 ligand (TNFSF8), OPG (TNFRSF11B), 4-1BB (TNFRSF9), 4-IBB ligand (TNFSF9), C1QBP, C1R, C1S, C3, C6, C7, CFH, CFP, SerpinC1 (antithrombin III), MBL2, F10 (Factor Xa), BMP7, NOG, DKK1, DKK4, SHH, TrkB (NTRK2), TrkC (NTRK3) or IgE.

62. The method of claim 59, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

63. A method for treating a condition or disease prevalent in a typical individual, the method comprising administering an effective amount of a pharmaceutical composition to the individual, wherein the pharmaceutical composition increases the expression or activity of a protein in Table 1 associated with the condition or disease prevelant in the typical individual.

64. The method of claim 63, wherein the protein is fibroblast growth factor receptor, neuropilin, matrix metalloproteinase, B2-microglobulin, Cystatin C, Annexin H, Notch protein, tumor necrosis factor (TNF) receptor, inosine 5′ monophosphate dehydrogenase, an insulin-like growth factor binding protein, Trefoil factor or endostatin.

65. The method of claim 63, wherein the protein is FGFR1, NRP1, MMP1, B2-microglobulin (B2M), Cystatin C (CST3), Annexin II (ANXA2), Notch3, TNF sR-1 (TNFRSF1A), TNF sR-2 (TNFRSF1B), TAJ (TNFRSF19), DR3 (TNFRSF25), IMPDH1, IMPDH2, TFF3, TFF1 Col18A1 or IGFBP6.

66. The method of claim 63, wherein the condition or disease comprises heart disease, cancer, stroke, coronary artery disease, atherosclerosis, hypertension and/or angiopathies.

67. A method for treating a condition or disease prevalent in a typical individual, the method comprising administering an effective amount of a pharmaceutical composition to the individual, wherein the pharmaceutical composition decreases the expression or activity of a protein in Table 2 associated with the condition or disease prevalent in the typical individual.

68. The method of claim 67, wherein the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE.

69. The method of claim 67, wherein the protein is EGFR, ERBB3, ERBB4, PDGFR, Notch1 CD30 (TNFRSF8), CD30 ligand (TNFSF8), OPG (TNFRSF11B), 4-1BB (TNFRSF9), 4-1BB ligand (TNFSF9), C1QBP, C1R, C1S, C3, C6, C7, CFH, CFP, SerpinC1 (antithrombin III), MBL2, F10 (Factor Xa), BMP7, NOG, DKK1, DKK4, SHH, TrkB (NTRK2), TrkC (NTRK3) or IgE,

70. The method of claim 67, wherein the condition or disease comprises heart disease, cancer, stroke, coronary artery disease, atherosclerosis, hypertension and/or angiopathies.

71. A method for treating a condition or disease prevalent in an individual with Down syndrome comprising administering an effective amount of a pharmaceutical composition to the individual, wherein the expression or activity level of a protein in Table 1 is higher than a predetermined threshold level, and wherein the effective amount of the pharmaceutical composition reduces the expression or activity level of the protein in the individual.

72. The method of claim 71, wherein the protein is fibroblast growth factor receptor, neuropilin, matrix metalloproteinase, B2-microglobulin, Cystatin C, Annexin II, Notch protein, tumor necrosis factor (TNF) receptor, inosine 5′ monophosphate dehydrogenase, an insulin-like growth factor binding protein, Trefoil factor or endostatin.

73. The method of claim 71, wherein the protein is FGFR1, NRP1, MMP1, B2-microglobulin (B2M), Cystatin C (CST3), Annexin H (ANXA2), Notch3, TNF sR-1 (TNFRSF1A), TNF sR-2 (TNFRSF1B), TAJ (TNFRSFI9), DR3 (TNFRSF25), IMPDH1, IMPDH2, TFF3, TFF1, Col18A1 or IGFBP6.

74. The method of claim 71, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

75. A method for treating a condition or disease prevalent in an individual with Down syndrome comprising administering an effective amount of a pharmaceutical composition to the individual, wherein the expression or activity level of a protein in Table 2 is lower than a predetermined threshold level, and wherein the effective amount of the pharmaceutical composition increases the expression or activity level of the protein in the individual.

76. The method of claim 75, wherein the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE.

77. The method of claim 75, wherein the protein is EGFR, ERBB3, ERBB4, PDGFR, Notch1, CD30 (TNFRSF8), CD30 ligand (TNFSF8), OPG (TNFRSF11B), 4-1BB (TNFRSF9), 4-IBB ligand (TNFSF9), C1QBP, C1R, C1S, C3, C6, C7, CFH, CFP, SerpinC1 (antithrombin III), MBL2, F10 (Factor Xa), BMP7, NOG, DKK1, DKK4, SHH. TrkB (NTRK2), TrkC (NTRK3) or IgE.

78. The method of claim 75, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

79. A method for treating a condition or disease prevalent in a typical individual comprising administering an effective amount of a pharmaceutical composition to the individual, wherein the expression or activity level of a protein in Table 1 is lower than a predetermined threshold level, and wherein the effective amount of the pharmaceutical composition increases the expression or activity level of the protein in the individual.

80. The method of claim 79, wherein the protein is fibroblast growth factor receptor, neuropilin, matrix metalloproteinase, B2-microglobulin, Cystatin C, Annexin H, Notch protein, tumor necrosis factor (TNF) receptor, inosine 5′ monophosphate dehydrogenase, an insulin-like growth factor binding protein, Trefoil factor or endostatin.

81. The method of claim 79, wherein the protein is FGFR1, NRP1, MMP1, B2-microglobulin (B2M), Cystatin C (CST3), Annexin II (ANXA2), Notch3, TNF sR-1 (TNFRSF1A), TNF sR-2 (TNFRSF1B), TAJ (TNFRSF19), DR3 (TNFRSF25), IMPDH1, IMPDH2, TFF3, TFF1 Col18A1 or IGFBP6.

82. The method of claim 79, wherein the condition or disease comprises heart disease, cancer, stroke, coronary artery disease, atherosclerosis, hypertension and/or angiopathies.

83. A method for treating a condition or disease prevalent in a typical individual comprising administering an effective amount of a pharmaceutical composition to the individual, wherein the expression or activity level of a protein in Table 2 is higher than a predetermined threshold level, and wherein the effective amount of the pharmaceutical composition decreases the expression or activity level of the protein in the individual.

84. The method of claim 83, wherein the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE.

85. The method of claim 83, wherein the protein is EGFR, ERBB3, ERBB4, PDGFR, Notch1 CD30 (TNFRSF8), CD30 ligand (TNFSF8), OPG (TNFRSF11B), 4-1BB (TNFRSF9), 4-1BB ligand (TNFSF9), C1QBP, C1R, C1S, C3, C6, C7, CFH, CFP, SerpinC1 (antithrombin III), MBL2, F10 (Factor Xa), BMP7, NOG, DKK1, DKK4, SHH, TrkB (NTRK2), TrkC (NTRK3) or IgE.

86. The method of claim 83, wherein the condition or disease comprises heart disease, cancer, stroke, coronary artery disease, atherosclerosis, hypertension and/or angiopathies.

87. A method for treating a condition or disease prevalent in an individual with Down syndrome, the method comprising:
(a) measuring the expression or activity level of a protein in Table 1 in a sample obtained from the individual;
(b) comparing the expression or activity level of the protein to a reference level in a normal control; and
(c) treating the individual with an effective amount of a pharmaceutical composition if the expression or activity level of the protein is increased relative to the reference level, wherein the pharmaceutical composition reduces the expression or activity of the protein.

88. The method of claim 87, further comprising step (d) of measuring the expression or activity of the protein in the individual after step (c) of treating the individual, wherein a decrease in the expression or activity of the protein is indicative that the patient is responsive to the treatment,

89. The method of claim 87, wherein the protein is fibroblast growth factor receptor, neuropilin, matrix metalloproteinase, B2-microglobulin, Cystatin C, Annexin II, Notch protein, tumor necrosis factor (TNF) receptor, inosine 5′ monophosphate dehydrogenase, an insulin-like growth factor binding protein, Trefoil factor or endostatin.

90. The method of claim 87, wherein the protein is FGFR1, NRP1, MMP1, B2-microglobulin (B2M), Cystatin C (CST3), Annexin II (ANXA2), Notch3, TNF sR-1 (TNFRSF1A), TNF sR-2 (TNFRSF1B), TAJ (TNFRSFI9), DR3 (TNFRSF25), IMPDH1, IMPDH2, TFF3, TFF1, Col18A1 or IGFBP6.

91. The method of claim 87, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

92. A method for treating a condition or disease prevalent in an individual with Down syndrome, the method comprising:
(a) measuring the expression or activity level of a protein in Table 2 in a sample obtained from the individual;
(b) comparing the expression or activity level of the protein to a reference level in a normal control; and
(c) treating the individual with an effective amount of a pharmaceutical composition if the expression or activity level of the protein is decreased relative to the reference level, wherein the pharmaceutical composition increases the expression or activity of the protein.

93. The method of claim 92, further comprising step (d) of measuring the expression or activity of the protein in the individual after step (c) of treating the individual, wherein an increase in the expression or activity of the protein is indicative that the patient is responsive to the treatment.

94. The method of claim 92, wherein the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE.

95. The method of claim 92, wherein the protein is EGFR, ERBB3, ERBB4, PDGFR, Notch1 CD30 (TNFRSF8), CD30 ligand (TNFSF8), OPG (TNFRSF11B), 4-1BB (TNFRSF9), 4-1BB ligand (TNFSF9), C1QBP, C1R, C1S, C3, C6, C7, CFH, CFP, SerpinC1 (antithrombin III), MBL2, F10 (Factor Xa), BMP7, NOG, DKK1, DKK4, SHH, TrkB (NTRK2), TrkC (NTRK3) or IgE,

96. The method of claim 92, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

97. A method for treating a condition or disease prevalent in a typical individual, the method comprising:
(a) measuring the expression or activity level of a protein in Table 1 in a sample obtained from the individual;
(b) comparing the expression or activity level of the protein to a reference level in a normal control; and
(c) treating the individual with an effective amount of a pharmaceutical composition if the expression or activity level of the protein is decreased relative to the reference level, wherein the pharmaceutical composition increases the expression or activity of the protein,

98. The method of claim 97, further comprising step (d) of measuring the expression or activity of the protein in the individual after step (c) of treating the individual, wherein an increase in the expression or activity of the protein is indicative that the patient is responsive to the treatment.

99. The method of claim 97, wherein the protein is fibroblast growth factor receptor, neuropilin, matrix metalloproteinase, B2-microglobulin, Cystatin C, Annexin II, Notch protein, tumor necrosis factor (TNF) receptor, inosine 5′ monophosphate dehydrogenase, an insulin-like growth factor binding protein, Trefoil factor or endostatin.

100. The method of claim 97, wherein the protein is FGFR1, NRP1, MMP1, B2-microglobulin (B2M), Cystatin C (CST3), Annexin II (ANXA2), Notch3, TNF sR-1 (TNFRSF1A), TNF sR-2 (TNFRSF1B), TAJ (TNFRSF19), DR3 (TNFRSF25), IMPDH1, IMPDH2, TFF3, TFF1 Col18A1 or IGFBP6.

101. The method of claim 97, wherein the condition or disease comprises heart disease, cancer, stroke, coronary artery disease, atherosclerosis, hypertension and/or angiopathies.

102. A method for treating a condition or disease prevalent in a typical individual, the method comprising:
(a) measuring the expression or activity level of a protein in Table 2 in a sample obtained from the individual;
(b) comparing the expression or activity level of the protein to a reference level in a normal control; and
(c) treating the individual with an effective amount of a pharmaceutical composition if the expression or activity level of the protein is increased relative to the reference level, wherein the pharmaceutical composition decreases the expression or activity of the protein.

103. The method of claim 102, further comprising step (d) of measuring the expression or activity of the protein in the individual after step (c) of treating the individual, wherein a decrease in the expression or activity of the protein is indicative that the patient is responsive to the treatment.

104. The method of claim 102, wherein the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE.

105. The method of claim 102, wherein the protein is EGFR, ERBB3, ERBB4, PDGFR, Notch1, CD30 (TNFRSFB), CD30 ligand (TNFSF8), OPG (TNFRSF11B), 4-1BB (TNFRSF9), 4-1BB ligand (TNFSF9), C1QBP, C1R, C1S, C3, C6, C7, CFH, CFP, SerpinC1 (antithrombin III), MBL2, F10 (Factor Xa), BMP7, NOG, DKK1, DKK4, SHH, TrkB (NTRK2), TrkC (NTRK3) or IgE.

106. The method of claim 102, wherein the condition or disease comprises heart disease, cancer, stroke, coronary artery disease, atherosclerosis, hypertension and/or angiopathies.

107. A method for treating a condition or disease prevalent in an individual with Down syndrome, the method comprising:
(a) determining the expression or activity level of a protein in Table 1 in a first sample obtained from the individual;
(b) determining the expression or activity level of the protein in a second sample from the individual, wherein the second sample is obtained after administration to the individual of an initial dosage of a pharmaceutical composition; and
(c) administering an adjusted dosage of the pharmaceutical composition, wherein the adjusted dosage is greater than the initial dosage if the expression or activity level of the protein in the second sample is greater than a predetermined threshold level.

108. The method of claim 107, wherein the protein is fibroblast growth factor receptor, neuropilin, matrix metalloproteinase, B2-microglobulin, Cystatin C, Annexin II, Notch protein, tumor necrosis factor (TNF) receptor, inosine 5′ monophosphate dehydrogenase, an insulin-like growth factor binding protein, Trefoil factor or endostatin.

109. The method of claim 107, wherein the protein is FGFR1, NRP1, MMPI, B2-microglobulin (B2M), Cystatin C (CST3), Annexin II (ANXA2), Notch3, TNF sR-1 (TNFRSF1A), TNF sR-2 (TNFRSF1B), TAJ (TNFRSF19), DR3 (TNFRSF25), IMPDH1, IMPDH2, TFF3, TFF1, Col18A1 or IGFBP6.

110. The method of claim 107, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

111. A method for treating a condition or disease prevalent in an individual with Down syndrome, the method comprising:
(a) determining the expression or activity level of a protein in Table 2 in a first sample obtained from the individual;
(b) determining the expression or activity level of the protein in a second sample from the individual, wherein the second sample is obtained after administration to the individual of an initial dosage of a pharmaceutical composition: and
(c) administering an adjusted dosage of the pharmaceutical composition, wherein the adjusted dosage is greater than the initial dosage if the expression or activity level of the protein in the second sample is reduced relative to a predetermined threshold level.

112. The method of claim 111, wherein the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE.

113. The method of claim 111, wherein the protein is EGFR, ERBB3, ERBB4, PDGFR, Notch1, CD30 (TNFRSF8), CD30 ligand (TNFSF8), OPG (TNFRSF11B), 4-1BB (TNFRSF9), 4-1BB ligand (TNFSF9), C1QBP, C1R, C1S, C3, C6, C7, CFH, CFP, SeminC1 (antithrombin III), MBL2, F10 (Factor Xa), BMP7, NOG, DKK1, DKK4, SHH, TrkB (NTRK2), TrkC (NTRK3) or IgE.

114. The method of claim 111, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

115. A method for treating a condition or disease prevalent in an individual with Down syndrome, the method comprising:
(a) administering a first dosage of a pharmaceutical composition;
(b) determining the expression or activity level of a protein in Table 1 in a sample obtained from the individual;
(c) determining whether the dosage of the pharmaceutical composition needs to be adjusted based on whether the expression or activity level of the protein falls within a target range, wherein an expression or activity level of the protein falling above the target range indicates that the dosage needs to be increased and an expression or activity level of the protein falling below a target range indicates that the dosage needs to be decreased, and
(d) administering a second dosage of the pharmaceutical composition based on the determination in (c).

116. The method of claim 115, wherein the protein is fibroblast growth factor receptor, neuropilin, matrix metalloproteinase, B2-microglobulin, Cystatin C, Annexin II, Notch protein, tumor necrosis factor (TNF) receptor, inosine 5′ monophosphate dehydrogenase, an insulin-like growth factor binding protein, Trefoil factor or endostatin.

117. The method of claim 115, wherein the protein is FGFR1, NRP1 MMPI , B2-microglobulin (B2M), Cystatin C (CST3), Annexin II (ANXA2), Notch?), TNF sR-1 (TNFRSF1A), TNF sR-2 (TNFRSF1B), TAJ (TNFRSF19), DR3 (TNFRSF25), IMPDH1 IMPDH2, TFF3, TFFI, Col18A1 or IGFBP6.

118. The method of claim 115, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

119. A method for treating a condition or disease prevalent in an individual with Down syndrome, the method comprising:
(a) administering a first dosage of a pharmaceutical composition;
(b) determining the expression or activity level of a protein in Table 2 in a sample obtained from the individual;
(c) determining whether the dosage of the pharmaceutical composition needs to be adjusted based on whether the expression or activity level of the protein falls within a target range, wherein an expression or activity level of the protein falling below the target range indicates that the dosage needs to be increased and an expression or activity level of the protein falling above a target range indicates that the dosage needs to be decreased, and
(d) administering a second dosage of the pharmaceutical composition based on the determination in (c).

120. The method of claim 119, wherein the protein is epidermal growth factor receptor, platelet-derived growth factor receptor, Notch protein, tumor necrosis factor (TNF) receptor, TNF receptor ligand, a protein associated with the complement cascade, a protein associated with the coagulation cascade, a protein associated with bone and limb development, a Wnt inhibitor, Sonic hedgehog, a neurotrophin receptor or IgE.

121. The method of claim 119, wherein the protein is EGFR, ERBB3, ERBB4, PDGFR, Notch1, CD30 (TNFRSF8), CD30 ligand (TNFSF8), OPG (TNFRSF11B), 4-1BB (TNFRSF9), 4-1BB ligand (TNFSF9), C1QBP, C1R, C1S, C3, C6, C7, CFH, CFP, SerpinC1 (antithrombin III), MBL2, F10 (Factor Xa), BMP7, NOG, DKK1, DKK4, SHH, TrkB (NTRK2), TrkC (NTRK3) or IgE.

122. The method of claim 119, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

123. A method for treating a condition or disease prevalent in an individual with Down syndrome comprising measuring the expression or activity level of a protein in Table 1 or Table 2 in the individual upon administration a first amount of a pharmaceutical composition, administering a second amount of the pharmaceutical composition to the patient, wherein the second amount of the pharmaceutical composition is determined based on the expression or activity level of the protein in Table 1 or Table 2.

124. The method of claim 123, wherein the protein is fibroblast growth factor receptor, epidermal growth factor receptor, platelet-derived growth factor receptor, neuropilin, matrix metalloproteinase, B2-microglobulin, Cystatin C, Annexin II, Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin.

125. The method of claim 123, wherein the protein is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B). TAJ (TNFRSF19), DR3 (TNFRSF25), 4-1BB (TNFRSF9), 4-IBB ligand (TNFSF9), IMPDH1, IMPDH2, C1QBP, C1R, C1S, C3, C6, C7, CFH, CFP, SerpinC1 (antithrombin III), MBL2, F10 (Factor Xa). BMP7, NOG, DKK1, DKK4, SHH, TFF3, TFF1, Col18A1, TrkB (NTRK2), TrkC (NTRK3), IGFBP6 or IgE.

126. The method of claim 123, wherein the condition or disease comprises Alzheimer's, cognitive impairment, epilepsy, leukemia, diabetes, autism, congenital heart defects, celiac disease, thyroid dysfunction, autoimmune disorders, vision problems, hearing problems, intestinal atresia and/or sleep apnea.

127. A method for treating a condition or disease prevalent in a typical individual comprising measuring the expression or activity level of a protein in Table 1 or Table 2 in the individual upon administration a first amount of a pharmaceutical composition, administering a second amount of the pharmaceutical composition to the patient, wherein the second amount of the pharmaceutical composition is determined based on the expression or activity level of the protein in Table 1 or Table 2.

128. The method of claim 127, wherein the protein is fibroblast growth factor receptor, epidermal growth factor receptor, platelet-derived growth factor receptor, neuropilin, matrix metalloproteinase, B2-microglobulin, Cystatin C, Annexin II, Notch protein, tumor necrosis factor (TNF) receptor, TNF receptor ligand, inosine 5′ monophosphate dehydrogenase, a protein associated with the complement cascade, a protein associated with the coagulation cascade, a protein associated with bone and limb development, a Wnt inhibitor, Sonic hedgehog, a neurotrophin receptor, an insulin-like growth factor binding protein, IgE, Trefoil factor or endostatin.

129. The method of claim 127, wherein the protein is FGFR1, EGFR, ERBB3, ERBB4, PDGFR, NRP1, MMP1, B2-microglobulin (B2M), Cystatin C (CST3), Annexin II (ANXA2), Notch1, Notch3, TNF sR-1 (TNFRSF1A), TNF sR-2 (TNFRSF1B), CD30 (TNFRSF8), CD30 ligand (TNFSF8), OPG (TNFRSF11B), TAJ (TNFRSF19), DR3 (TNFRSF25), 4-1BB (TNFRSF9), 4-1BB ligand (TNFSF9), IMPDH1, IMPDH2, C1QBP, C1R, C1S, C3, C6, C7, CFH, CFP, SerpinC1 (antithrombin III), MBL2, F10 (Factor Xa), BMP7, NOG, DKK1, DKK4, SHH, TFF3, TFF1, Col18A1, TrkB (NTRK2), TrkC (NTRK3), IGFBP6 or IgE.

130. The method of claim 127, wherein the condition or disease comprises heart disease, cancer, stroke, coronary artery disease, atherosclerosis, hypertension and/or angiopathies.
